SEQUENCE LISTING (33 sequences)

SEQ ID NO: 1 - DNA, 52 bp, synthetic construct
  source 1..52: mol_type = other DNA
gttaaattatctaggcattcgattcaagagatcgaatgcctagataatttaa

SEQ ID NO: 2 - RNA, 25 nt, synthetic construct
  source 1..25: mol_type = other RNA
atttaaattatctaggcattcgaaa

SEQ ID NO: 3 - DNA, 30 bp, synthetic construct
  source 1..30: mol_type = other DNA
atatatatatttcaaggatataccattcta

SEQ ID NO: 4 - DNA, 242 bp, synthetic construct
  source 1..242: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 5 - DNA, 313 bp, synthetic construct
  source 1..313: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 6 - DNA, 232 bp, synthetic construct
  source 1..232: mol_type = other DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 7 - DNA, 3802 bp, synthetic construct
  source 1..3802: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatagaattctatctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggccagcttcccacaataagttgggtgaattttggctcattcctcctttctataggattgaggtcagagctttgtgatgggaattctgtggaatgtgtgtcagttagggtgtggaaagtcccgcgatcgctcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccgttaaattatctaggcattcgattcaagagatcgaatgcctagataatttaattttttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggccggcctgcatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttccctgcaggtgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtcccttagcggccgctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 8 - DNA, 5764 bp, synthetic construct
  source 1..5764: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatagaattctatctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggccagcttcccacaataagttgggtgaattttggctcattcctcctttctataggattgaggtcagagctttgtgatgggaattctgtggaatgtgtgtcagttagggtgtggaaagtcccgcgatcgctcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccgttaaattatctaggcattcgattcaagagatcgaatgcctagataatttaattttttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggccggcctgcatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttccctgcaggtgtcctcacaggaacgaagtccctaaagaaacagtggcagccaggtttagccccggaattgactggattccttttttagtcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccgttaaattatctaggcattcgattcaagagatcgaatgcctagataatttaattttttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggtcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccgttaaattatctaggcattcgattcaagagatcgaatgcctagataatttaattttttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtcccttagcggccgctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 9 - DNA, 6619 bp, synthetic construct
  source 1..6619: mol_type = other DNA
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccgttaaattatctaggcattcgattcaagagatcgaatgcctagataatttaattttttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 10 - DNA, 1785 bp, synthetic construct
  source 1..1785: mol_type = other DNA
atgggttcctccttggatgatgaacatattttgtctgccttgttgcaatccgatgatgaattggttggtgaagattccgattccgaagtttctgatcatgtttccgaagatgatgttcaatctgatactgaagaagccttcatcgacgaagttcatgaagttcaacctacatcttccggttccgaaattttggatgaacaaaacgttatcgaacagccaggttcttcattggcttctaacagaattttgaccttgccacaaagaaccatcaggggtaaaaacaaacattgctggtctacttctaagtccaccagaagatcaagagtttctgctttgaacatcgtcagatctcaaaggggtccaactagaatgtgtagaaatatctacgatcccctgttgtgcttcaagttgttttttaccgacgaaatcatctccgaaatcgttaagtggactaacgccgaaatttccttgaagagaagagaatctatgacctctgctactttcagagatacaaacgaagatgagatctacgccttcttcggtattttggttatgactgctgttagaaaggacaaccatatgtctaccgatgatttgttcgacaggtctttgtctatggtttacgtgtctgttatgtccagagatagattcgacttcttgatcagatgcttgagaatggatgacaagtctattagaccaaccttgagagaaaacgatgttttcactccagttagaaagatctgggacttgttcattcatcaatgcatccaaaactataccccaggtgctcatttgactattgatgaacaactgttgggtttcagaggtagatgtcctttcagagtttacattccaaacaagccatctaagtacggcatcaagattttgatgatgtgtgactctggtacgaagtacatgattaacggtatgccatacttaggtagaggtactcaaactaatggtgttccattgggtgagtactacgtcaaagaattgtctaaaccagttcacggttcctgcagaaacattacttgtgataattggttcacctccattccattggctaagaatttgttgcaagagccatacaagttgaccatcgttggtactgttagatccaacaagagagaaatcccagaagtcttgaagaacagtagatcaagaccagttggtacttctatgttctgttttgatggtccattgaccttggtttcttacaaacctaaaccagccaagatggtgtacttgttgtcatcttgtgatgaagatgcctccattaacgaatctactggtaaaccacaaatggtcatgtactacaatcagaccaaaggtggtgttgataccttggatcaaatgtgttctgttatgacctgttccagaaagactaatagatggccaatggctttgttgtacggcatgatcaatattgcctgtatcaactccttcatcatctactcccataacgtttcttctaagggtgaaaaggtccagtccagaaaaaagtttatgaggaacctgtatatgtccctgacctcttcattcatgagaaagagattggaagctccaactttgaagagatacttgagagacaacatctccaacatcttgcctaaagaagttccaggtacatctgatgattcaaccgaagaaccagttatgaagaagagaacttactgtacctactgtccctccaaaattagacgtaaagctaacgcttcttgtaagaagtgcaagaaggttatctgcagagaacacaacattgacatgtgtcaatcctgcttctag

SEQ ID NO: 11 - DNA, 644 bp, synthetic construct
  source 1..644: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcga

SEQ ID NO: 12 - DNA, 248 bp, synthetic construct
  source 1..248: mol_type = other DNA
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 13 - DNA, 269 bp, synthetic construct
  source 1..269: mol_type = other DNA
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatc

SEQ ID NO: 14 - DNA, 100 bp, synthetic construct
  source 1..100: mol_type = other DNA
tttattttttgtcactattgttatgtaaaatgccacctctgacagtatggaacgcaaacttctgtctagtggataacagaatttttctatggccaattta

SEQ ID NO: 15 - DNA, 405 bp, synthetic construct
  source 1..405: mol_type = other DNA
aactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattcta

SEQ ID NO: 16 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 17 - DNA, 20 bp, synthetic construct
  source 1..20: mol_type = other DNA
cttgtcatctaaacccacac

SEQ ID NO: 18 - DNA, 20 bp, synthetic construct
  source 1..20: mol_type = other DNA
gtatagaaccgtggatgatg

SEQ ID NO: 19 - DNA, 20 bp, synthetic construct
  source 1..20: mol_type = other DNA
gatatcaccaaacatgttgc

SEQ ID NO: 20 - DNA, 20 bp, synthetic construct
  source 1..20: mol_type = other DNA
tccgttatcccaggttcctt

SEQ ID NO: 21 - DNA, 618 bp, synthetic construct
  source 1..618: mol_type = other DNA
acatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 22 - DNA, 60 bp, synthetic construct
  source 1..60: mol_type = other DNA
atcggttctttccaatttttttttttttttttttttttttttttttttttggttgtaata

SEQ ID NO: 23 - DNA, 60 bp, synthetic construct
  source 1..60: mol_type = other DNA
aagaataaacatttatctgatatgtaattgcatttataaaatgtacagtaccgcatttaa

SEQ ID NO: 24 - DNA, 60 bp, synthetic construct
  source 1..60: mol_type = other DNA
aagaaatatatagatttaggtattcgttaaatatatacacattaaatggcctcagaaatt

SEQ ID NO: 25 - DNA, 60 bp, synthetic construct
  source 1..60: mol_type = other DNA
atatataaataaataagctcttatatgtacaaatttgtgcatatacttttcttgaccttt

SEQ ID NO: 26 - DNA, 60 bp, synthetic construct
  source 1..60: mol_type = other DNA
agtctaagctgaaagattattactttcatttgatttttttatttttgaagccccatttcc

SEQ ID NO: 27 - DNA, 60 bp, synthetic construct
  source 1..60: mol_type = other DNA
atcgttctcgtggacgagattaaaaatagaaatgatgtagaggagatgcactaaacattg

SEQ ID NO: 28 - DNA, 60 bp, synthetic construct
  source 1..60: mol_type = other DNA
taacaattgataaattatttgaagtatcttccaagacttcaaacaaagatattttcaagt

SEQ ID NO: 29 - DNA, 60 bp, synthetic construct
  source 1..60: mol_type = other DNA
aaaggttgtgaagtcaactgttcaagacatgactggcaaaggaaactttatgcatctatc

SEQ ID NO: 30 - DNA, 60 bp, synthetic construct
  source 1..60: mol_type = other DNA
tttttcttggcctcgaagaaatttcgagataccttgctcgtaaccctcccagaagtttcc

SEQ ID NO: 31 - DNA, 60 bp, synthetic construct
  source 1..60: mol_type = other DNA
tattactgtagtccccacgggacaagataccttgtacctttttccattggtaataccacc

SEQ ID NO: 32 - DNA, 60 bp, synthetic construct
  source 1..60: mol_type = other DNA
gaataacggaaaaggagcctgcagccagactgtagaaagatgacactgccaagagaataa

SEQ ID NO: 33 - DNA, 60 bp, synthetic construct
  source 1..60: mol_type = other DNA
aagaaaaaacaccccaaacaccctgaccggcggcgaagcccctctgcgcgctcaacgcgt

(19) United States
(12) Patent Application Publication
(10) Pub. No.: US 2025/0017220 A1
(43) Pub. Date: Jan. 16, 2025

(54) YEAST STRAIN FOR MOSQUITO MANAGEMENT

(71) Applicants: The Trustees of Indiana University, Bloomington, IN (US); Demeetra Agbio, Inc., Lexington, KY (US)

(72) Inventors: Molly Duman Scheel, Niles, MI (US); Keshava Mysore, South Bend, IN (US); Corey Brizzee, Lexington, KY (US); Jack Crawford, Lexington, KY (US)

(73) Assignees: The Trustees of Indiana University, Bloomington, IN (US); Demeetra Agbio, Inc., Lexington, KY (US)

(21) Appl. No.: 18/771,754

(22) Filed: Jul. 12, 2024

Related U.S. Application Data
(60) Provisional application No. 63/526,834, filed on Jul. 14, 2023.

(51) Int. Cl. A01N 63/32; A01P 7/04; C12N 1/16; C12N 15/113; C12N 15/81; C12R 1/84; C12R 1/865

(52) U.S. Cl. CPC A01N 63/32; A01P 7/04; C12N 1/16; C12N 15/113; C12N 15/815; C12R 2001/84; C12R 2001/865

(57) ABSTRACT

Disclosed herein are methods for producing interfering RNA biopesticides, such as microbial host organisms engineered to produce interfering RNA molecules which inhibit the expression of a gene in a mosquito disease vectors by RNA interference. Also disclosed herein are polynucleotides, such as expression cassettes encoding such interfering RNA molecule and facilitating integration, such as stable integration into the host organism's genome. Further disclosed herein are compositions including the disclosed nucleotide sequences and host organisms, along with methods of using the same to control mosquito populations.

### CROSS-REFERENCE TO RELATED APPLICATION

The present application claims priority to U.S. Provisional Application No. 63/526,834, filed on Jul. 14, 2023, and entitled “YEAST STRAIN FOR MOSQUITO MANAGEMENT,” the entire disclosure of which is expressly incorporated by reference herein.

### REFERENCE TO SEQUENCE LISTING

The instant application contains a Sequence Listing XML which has been submitted electronically and is hereby incorporated by reference in its entirety. Said Sequence Listing XML copy, created on Jun. 26, 2024 is named “IU202301602WOST26.xml” and is 51,544 bytes in size.

### FIELD OF THE INVENTION

This disclosure generally relates to yeast-based insecticide compositions which can be deployed to control, such as reduce, populations of disease vectors, such as mosquitoes. The disclosure further relates to methods of making the yeast-based insecticide compositions.

### BACKGROUND

Mosquito-borne illnesses, e.g., malaria and Dengue virus, are a major threat to human health and detrimentally impact economic growth. Given poor progress in vaccine development and distribution, mosquito population control is the primary mechanism for disease prevention. However, the existing arsenal for eliminating mosquitoes is inadequate. For example, insecticide resistance is increasing, rendering some currently used options useless. Additionally, concern for the off-target effects of pesticides is increasing. For these and other reasons, the current pesticide repertoire is insufficient for combating existing and future vector-borne diseases, resulting in an urgent need to develop a new generation of environmentally safe options for mosquito control.

Given its sequence dependent mechanism of action, RNA interference provides a more selective alternative to broad-spectrum insecticides, and the technology has been shown to undermine the growth, such as the fitness, and survival of targeted insect pests, such as mosquitoes. However, delivery of interfering RNA remains challenging, and various strategies include administration by injection of naked double stranded RNA (dsRNA) and oral administration, such as by combining insecticidal nucleic acid molecules with artificial diet.

An emerging strategy involves facilitating exposure of mosquitoes to symbiotic or attractive microbes, such as yeast, engineered to produce insecticidal nucleic acid molecules, such as interfering RNA. Efficient and stable integration of expression cassettes into the microbial genome influences expression levels of desired nucleotide sequences and commercial feasibility. New approaches may be leveraged to optimize microbial production of insecticidal nucleic acid molecules, along with other features, in the application of engineered microbes as disease vector control. Accordingly, there exists a need for improved methods of engineering microbes to biosynthesize insecticidal nucleic acids, such as to generate microbes with advantageous capabilities, e.g., improved genetic stability and enhanced production of nucleic acids targeting mosquito genes. Additionally, there exists a need to provide engineered microbial strains suitable for scaled fermentation and commercial applications. Aspects of the invention disclosed herein address these needs.

### INCORPORATION BY REFERENCE

Each patent, publication, and non-patent literature cited in the application is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art.

### SUMMARY OF THE INVENTION

A first aspect of the invention includes prototrophic mutant yeast cells having an expression cassette stably integrated into at least one location within the yeast cell genome, where the expression cassette includes a nucleotide sequence encoding an interfering RNA molecule capable inhibiting gene expression in a mosquito, and where the nucleotide sequence encoding the interfering RNA molecule is flanked by an inverted terminal repeat sequence.

A second aspect of the invention includes expression cassettes containing a nucleotide sequence encoding an interfering RNA molecule capable inhibiting gene expression in a mosquito, where the nucleotide sequence encoding the interfering RNA molecule is flanked by an inverted terminal repeat sequence.

A third aspect of the invention includes compositions containing a yeast cell or an expression cassette of the aforementioned aspects, such as an expression cassette containing a nucleotide sequence encoding an interfering RNA molecule capable inhibiting gene expression in a mosquito, where the nucleotide sequence encoding the interfering RNA molecule is flanked by an inverted terminal repeat sequence, or a prototrophic mutant yeast cell having such expression cassette stably integrated into its genome.

A fourth aspect of the invention includes methods of engineering a microbial host cell to produce interfering RNA molecules, such as with use of a transposase/transposon system to stably integrate an expression cassette including a nucleotide sequence encoding an interfering RNA molecule into the genome of the host organism in addition to a nuclease system which is used to generate a deletion mutation in an auxotrophic rescue gene in the host organism.

A fifth aspect of the invention includes methods for controlling a mosquito population, such as use of the yeast cells, expression cassettes, and compositions described in various aspects of the invention as a mosquito biopesticide, such as an interfering RNA biopesticide targeting mosquitoes, for example, by feeding the yeast cells, expression cassettes, and compositions disclosed herein to a mosquito, and thereby undermining the fitness and/or the survival of the mosquito.

A first embodiment is a prototrophic mutant yeast cell having an expression cassette stably integrated into at least one location within the genome of the yeast cell, where the expression cassette includes a nucleotide sequence encoding an interfering RNA molecule; and where the interfering RNA molecule is capable of inhibiting gene expression of Shaker in a mosquito; and where the nucleotide sequence encoding the interfering RNA molecule is flanked by an inverted terminal repeat sequence.

A second embodiment is a prototrophic yeast cell which has an expression cassette stably integrated into the genome of the yeast cell, which includes a nucleotide sequence encoding an interfering RNA molecule, where the nucleotide sequence encoding the interfering RNA molecule is flanked by a 5′ inverted terminal repeat sequence and a 3′ inverted terminal repeat sequence.

A third embodiment is a prototrophic yeast cell which has an expression cassette stably integrated into the genome of the yeast cell, which contains a nucleotide sequence encoding an interfering RNA molecule, where the nucleotide sequence encoding the interfering RNA molecule is operably linked to a glyceraldehyde-3-phosphate dehydrogenase (GAP) promoter, where the GAP promoter separates the nucleotide sequence encoding the interfering RNA molecule from the 3′ inverted terminal repeat sequence.

A fourth embodiment is a prototrophic yeast cell which has an expression cassette stably integrated into the genome of the yeast cell, which includes a nucleotide sequence encoding an interfering RNA molecule, where the nucleotide sequence encoding the interfering RNA molecule is flanked on the 3′ end by a CYC1 terminator, where the CYC1 terminator separates the nucleotide sequence encoding the interfering RNA molecule from the 5′ inverted terminal repeat sequence.

A fifth embodiment is a prototrophic yeast cell which has an expression cassette stably integrated into the genome of the yeast cell, which includes a GAP promoter and a CYC1 terminator, where the GAP promoter includes a nucleotide sequence with at least 85% sequence identity to SEQ ID NO:11 and the CYC1 terminator includes a nucleotide sequence with at least 85% sequence identity to SEQ ID NO:12.

A sixth embodiment is a prototrophic yeast cell which has an expression cassette stably integrated into the genome of the yeast cell, where the expression cassette further includes an auxotrophic rescue promoter operably linked to an auxotrophic rescue gene, and where the auxotrophic rescue promoter is flanked by the 5′ inverted terminal repeat sequence.

A seventh embodiment is a prototrophic yeast cell which has an expression cassette including an auxotrophic rescue promoter stably integrated into the genome of the yeast cell, where the auxotrophic rescue promoter is a minimal auxotrophic rescue promoter.

An eighth embodiment is a prototrophic yeast cell which has an expression cassette including a minimal auxotrophic rescue promoter stably integrated into the genome of the yeast cell, where the minimal auxotrophic rescue promoter includes a nucleotide sequence with at least 70% identity to the entire length of SEQ ID NO:3.

A ninth embodiment is a prototrophic yeast cell which has an expression cassette including a minimal auxotrophic rescue promoter stably integrated into the genome of the yeast cell, where, where the minimal auxotrophic rescue promoter includes a nucleotide sequence with 100% identity to the entire length of SEQ ID NO:3.

A tenth embodiment is a prototrophic yeast cell which has an expression cassette including an auxotrophic rescue gene stably integrated into the genome of the yeast cell, where the auxotrophic rescue gene encodes leucine.

An eleventh embodiment is a prototrophic yeast cell which has an expression cassette including an auxotrophic rescue gene stably integrated into the genome of the yeast cell, where the auxotrophic rescue gene is leu2.

A twelfth embodiment is a prototrophic yeast cell which has an expression cassette including an auxotrophic rescue promoter stably integrated into the genome of the yeast cell, where the auxotrophic rescue promoter comprises SEQ ID NO:3, and the auxotrophic rescue gene is leu2.

A thirteenth embodiment is a prototrophic yeast cell which has an expression cassette further including an insulator sequence stably integrated into the genome of the yeast cell.

A fourteenth embodiment is a prototrophic yeast cell which has an expression cassette including an insulator sequence stably integrated into the genome of the yeast cell, where the insulator sequence separates the 5′ end of the nucleotide sequence encoding the interfering RNA molecule from the 3′ inverted terminal repeat sequence.

A fifteenth embodiment is a prototrophic yeast cell which has an expression cassette including an insulator sequence stably integrated into the genome of the yeast cell, where the insulator sequence separates the 3′ end of the nucleotide sequence encoding the interfering RNA molecule from the 5′ inverted terminal repeat sequence.

A sixteenth embodiment is a prototrophic yeast cell which has an expression cassette including an insulator sequence stably integrated into the genome of the yeast cell, where the insulator sequence has 70% identity to the entire length of SEQ ID NO:6.

A seventeenth embodiment is a prototrophic yeast cell which has an expression cassette including an insulator sequence stably integrated into the genome of the yeast cell, where the insulator sequence has 100% identity to the entire length of SEQ ID NO:6.

An eighteenth embodiment is a prototrophic yeast cell which has an expression cassette including a 5′ inverted terminal repeat sequence stably integrated into the genome of the yeast cell, where the 5′ inverted terminal repeat sequence has at least about 70% identity to the entire length of SEQ ID NO:5

A nineteenth embodiment is a prototrophic yeast cell which has an expression cassette including a 5′ inverted terminal repeat sequence stably integrated into the genome of the yeast cell, where the 5′ inverted terminal repeat sequence has 100% identity to the entire length of SEQ ID NO:5.

A twentieth embodiment is a prototrophic yeast cell which has an expression cassette including a 5′ inverted terminal repeat sequence stably integrated into the genome of the yeast cell, where the 5′ inverted terminal repeat sequence consists of the entire length of SEQ ID NO:5.

A twenty-first embodiment is a prototrophic yeast cell which has an expression cassette including a 3′ inverted terminal repeat sequence stably integrated into the genome of the yeast cell, where the 3′ inverted terminal repeat sequence has at least about 70% identity to the entire length of SEQ ID NO:4

A twenty-second embodiment is a prototrophic yeast cell which has an expression cassette including a 3′ inverted terminal repeat sequence stably integrated into the genome of the yeast cell, where the 3′ inverted terminal repeat sequence has 100% identity to the entire length of SEQ ID NO:4.

A twenty-third embodiment is a prototrophic yeast cell which has an expression cassette including a 3′ inverted terminal repeat sequence stably integrated into the genome of the yeast cell, where the 3′ inverted terminal repeat sequence consists of the entire length of SEQ ID NO:4.

A twenty-fourth embodiment is a prototrophic yeast cell which has a complemented auxotrophic mutation.

A twenty-fifth embodiment is a prototrophic yeast cell having a complemented auxotrophic mutation, where the complemented auxotrophic mutation includes his3Δ0, leu2Δ0, trp1Δ0, ura3Δ0, or a combination thereof.

A twenty-sixth embodiment is a prototrophic yeast cell which has an expression cassette stably integrated into the genome of the yeast cell, where the expression cassette includes at least one, at least three, or at least five copies of the nucleotide sequence encoding the interfering RNA molecule.

A twenty-seventh embodiment is a prototrophic yeast cell which has an expression cassette stably integrated into the genome of the yeast cell, where the expression cassette has one, two, three, four, five, six, seven, eight, nine, or ten locations of integration into the yeast cell genome.

A twenty-eighth embodiment is a prototrophic yeast cell which has an expression cassette including a nucleotide sequence encoding the interfering RNA molecule stably integrated into the genome of the yeast cell, where a nucleotide sequence transcribed from the nucleotide sequence encoding the interfering RNA molecule includes at least 25 contiguous nucleotides which are partially or perfectly complementary to a sequence including at least 80%, 84%, 88%, 92%, 96%, or 100% identity to the entire length of SEQ ID NO:2.

A twenty-ninth embodiment is a prototrophic yeast cell which has an expression cassette including a nucleotide sequence encoding an interfering RNA molecule stably integrated into the genome of the yeast cell, where the nucleotide sequence transcribed from the nucleotide sequence encoding the interfering RNA molecule includes at least 25 contiguous nucleotides which are partially or perfectly complementary to the entire length of SEQ ID NO:2.

A thirtieth embodiment is a prototrophic yeast cell which has an expression cassette including a nucleotide sequence encoding an interfering RNA molecule stably integrated into the genome of the yeast cell, where the nucleotide sequence encoding the interfering RNA molecule has at least 80% or at least 90% sequence similarity to the entire length of SEQ ID NO. 1.

A thirty-first embodiment is a prototrophic yeast cell which has an expression cassette including a nucleotide sequence encoding an interfering RNA molecule stably integrated into the genome of the yeast cell, where the at least 10% or least 20% sequence dissimilarity is within the nucleotide sequence TTCAAGAGA of SEQ ID NO:1.

A thirty-second embodiment is a prototrophic yeast cell which has an expression cassette including a nucleotide sequence encoding an interfering RNA molecule stably integrated into the genome of the yeast cell, where the nucleotide sequence encoding the interfering RNA molecule includes the entire length of SEQ ID NO. 1.

A thirty-third embodiment is a prototrophic yeast cell which has an expression cassette stably integrated into the genome of the yeast cell, where the expression cassette includes a nucleotide sequence with at least 70%, 80%, or 90% identity to the entire length of SEQ ID NO:7, or the complement thereof, or SEQ ID NO:8 or the complement thereof.

A thirty-fourth embodiment is a prototrophic yeast cell which has an expression cassette stably integrated into the genome of the yeast cell, where the expression cassette consists of a nucleotide sequence with 100% identity to the entire length of SEQ ID NO:7, or the complement thereof, or SEQ ID NO:8, or the complement thereof.

A thirty-fifth embodiment is a prototrophic yeast cell having an expression cassette including the entire length of SEQ ID NO:7, where the expression cassette has a single copy of a nucleotide sequence encoding an interfering RNA molecule, where the nucleotide sequence encoding the interfering RNA molecule includes the entire length of SEQ ID NO:1, where the interfering RNA molecule is capable of inhibiting gene expression of Shaker in a mosquito.

A thirty-sixth embodiment is a prototrophic yeast cell having an expression cassette including the entire length of SEQ ID NO:8, where the expression cassette has at least three copies of a nucleotide sequence encoding an interfering RNA molecule, where the nucleotide sequence encoding the interfering RNA molecule includes SEQ ID NO:1, the interfering RNA molecule is capable of inhibiting gene expression of Shaker in a mosquito, and the expression cassette has one genomic integration site.

A thirty-seventh embodiment is a prototrophic yeast cell having a nucleotide sequence encoding an interfering RNA molecule, including the entire length of SEQ ID NO:1, where the nucleotide sequence encoding the interfering RNA molecule is integrated at any one or more of genomic sites chromosome IV (NC_001136 at position 543,705), chromosome IV (NC_001136 position 1,357,520), chromosome VIII (NC_001140 position 124,029), chromosome X (NC_001142 position 181,309), chromosome XI (NC_001143 position 300,654), or chromosome XII (NC_001144 position 213,991).

A thirty-eighth embodiment is a prototrophic yeast cell with the genotype MATa, PiggyBac (leu2d/P_{TDH3}-shRNA 463-T_{CYC1}), CEN/ARS (URA3/SPBase_Sc-CO).

A thirty-ninth embodiment is a prototrophic yeast cell, where the nucleotide sequence encoding shRNA_463 is integrated on chromosomes IV (NC_001136 position 1,357,520), VIII (NC_001140 position 124,029), X (NC_001142 position 181,309), XI (NC_001143 position 300,654), and XII (NC_001144 position 213,991).

A fortieth embodiment is a prototrophic yeast cell including the genotype MATa, PiggyBac (leu2d/P_{TDH3}-shRNA 463-T_{CYC1}, P_{TDH3}-shRNA 463-T_{CYC1}, P_{TDH3}-shRNA 463-T_{CYC1}), CEN/ARS (URA3/SPBase-Sc-CO).

A forty-first embodiment is a prototrophic yeast cell, where the nucleotide sequence encoding shRNA_463 is integrated on chromosome IV (NC_001136) at position 543,705.

A forty-second embodiment is a prototrophic yeast cell, where the nucleotide sequence encoding shRNA_463 has at least 80% or at least 90% sequence similarity to the entire length of SEQ ID NO. 1

A forty-third embodiment is a prototrophic yeast cell, where the nucleotide sequence encoding shRNA_463 includes the entire length of SEQ ID NO. 1.

A forty-fourth embodiment is a prototrophic yeast cell, where the nucleotide sequence encoding shRNA_463 consists of the entire length of SEQ ID NO. 1.

A forty-fifth embodiment is a prototrophic yeast cell, where the yeast cell is Pichia pastoris, Saccharomyces cerevisiae, or Yarrowia lipolytica.

A forty-sixth embodiment is a prototrophic yeast cell, where the yeast cell is Saccharomyces cerevisiae strain FL100 or S288C.

A forty-seventh embodiment is a prototrophic yeast cell, such as an interfering RNA biopesticide targeting a gene in a mosquito, where the mosquito is a species of Aedes, Anopheles, or Culex.

A forty-eighth embodiment is a prototrophic yeast cell, such as an interfering RNA biopesticide targeting a gene in a mosquito, where the mosquito is Anopheles gambiae, Aedes aegypti, Aedes albopictus, Culex pipiens, or Culex quinquesfasciatus.

A forty-ninth embodiment is a prototrophic yeast cell, where 72 hours of growth in a high-cell density fermentation media yields at least 15 grams per liter of dry cell weight.

A fiftieth embodiment is a prototrophic yeast cell, where the yeast reaches an OD600 of at least 50 after 24 hours of growth in the fermentation media.

A fifty-first embodiment is a prototrophic yeast cell, where the yeast reaches an OD600 of at least 75 after 72 hours of growth in the fermentation media.

A fifty-second embodiment is a prototrophic yeast cell, where the expression level of the interfering RNA molecule increases by at least 100% after 72 hours of growth in the fermentation media relative to time=0.

A fifty-third embodiment is use of the prototrophic yeast cell of any embodiments as a mosquito biopesticide.

A fifty-fourth embodiment is an expression cassette including a nucleotide sequence encoding an interfering RNA molecule which is capable of inhibiting gene expression of Shaker in a mosquito, where the nucleotide sequence encoding the interfering RNA molecule is flanked by an inverted terminal repeat sequence.

A fifty-fifth embodiment is an expression cassette, where the interfering RNA molecule is an RNA construct, a double stranded RNA (dsRNA), a small interfering RNA (siRNA), a short hairpin RNA (shRNA), or an anti-sense oligonucleotide.

A fifty-sixth embodiment is an expression cassette, where the nucleotide sequence encoding the interfering RNA molecule is flanked by a 5′ inverted terminal repeat sequence and a 3′ inverted terminal repeat sequence.

A fifty-seventh embodiment is an expression cassette, which further includes a glyceraldehyde-3-phosphate dehydrogenase (GAP) promoter, where the GAP promoter is operably linked to the nucleotide sequence encoding the interfering RNA molecule; and where the GAP promoter separates the nucleotide sequence encoding the interfering RNA molecule from the 3′ inverted terminal repeat sequence.

A fifty-eighth embodiment is an expression cassette, which further includes aa CYC1 terminator, where the CYC1 terminator flanks the nucleotide sequence encoding the interfering RNA molecule on the 3′ end; and where the CYC1 terminator separates the nucleotide sequence encoding the interfering RNA molecule from the 5′ inverted terminal repeat sequence.

A fifty-ninth embodiment is an expression cassette, where the GAP promoter includes a nucleotide sequence with at least 85% sequence identity to SEQ ID NO:11 and the CYC1 terminator includes a nucleotide sequence with at least 85% sequence identity to SEQ ID NO:12.

A sixtieth embodiment is an expression cassette, which further includes an auxotrophic rescue promoter operably linked to an auxotrophic rescue gene, where the auxotrophic rescue promoter is flanked by the 5′ inverted terminal repeat sequence.

A sixty-first embodiment is an expression cassette, where the auxotrophic rescue promoter is a minimal auxotrophic rescue promoter.

A sixty-second embodiment is an expression cassette, where the minimal auxotrophic rescue promoter includes a nucleotide sequence with at least 70% identity to the entire length of SEQ ID NO:3.

A sixty-third embodiment is an expression cassette, where the minimal auxotrophic rescue promoter includes a nucleotide sequence with 100% identity to the entire length of SEQ ID NO:3.

A sixty-fourth embodiment is an expression cassette, where the auxotrophic rescue gene encodes histidine, leucine, tryptophan, or uracil.

A sixty-fifth embodiment is an expression cassette, where the auxotrophic rescue gene(s) is/are leu2, ura3, his3, trp1, or a combination thereof.

A sixty-sixth embodiment is an expression cassette, where the auxotrophic rescue promoter is dleu2 (SEQ ID NO:3) the auxotrophic rescue gene is leu2.

A sixty-seventh embodiment is an expression cassette, where the expression cassette further includes an insulator sequence.

A sixty-eighth embodiment is an expression cassette, where the insulator sequence separates the 5′ end of the nucleotide sequence encoding the interfering RNA molecule from the 3′ inverted terminal repeat sequence.

A sixty-ninth embodiment is an expression cassette, where the insulator sequence separates the 3′ end of the nucleotide sequence encoding the interfering RNA molecule from the 5′ inverted terminal repeat sequence.

A seventieth embodiment is an expression cassette, where the insulator sequence has 70% identity to the entire length of SEQ ID NO:6.

A seventy-first embodiment is an expression cassette, where the insulator sequence has 100% identity to the entire length of SEQ ID NO:6.

A seventy-second embodiment is an expression cassette, where the 5′ inverted terminal repeat sequence has at least about 70% identity to the entire length of SEQ ID NO:5.

A seventy-third embodiment is an expression cassette, where the 5′ inverted terminal repeat sequence has 100% identity to the entire length of SEQ ID NO:5.

A seventy-fourth embodiment is an expression cassette, where the 5′ inverted terminal repeat sequence consists of the entire length of SEQ ID NO:5.

A seventy-fifth embodiment is an expression cassette, where the 3′ inverted terminal repeat sequence has at least about 70% identity to the entire length of SEQ ID NO:4.

A seventy-sixth embodiment is an expression cassette, where the 3′ inverted terminal repeat sequence has 100% identity to the entire length of SEQ ID NO:4.

A seventy-seventh embodiment is an expression cassette, where the 3′ inverted terminal repeat sequence consists of the entire length of SEQ ID NO:4.

A seventy-eighth embodiment is an expression cassette, where the expression cassette includes at least one, at least three, or at least five copies of the nucleotide sequence encoding the interfering RNA molecule.

A seventy-ninth embodiment is an expression cassette, where a nucleotide sequence transcribed from the nucleotide sequence encoding the interfering RNA molecule includes at least 25 contiguous nucleotides which are partially or perfectly complementary to a sequence including at least 80%, 84%, 88%, 92%, 96%, or 100% identity to the entire length of SEQ ID NO:2.

An eightieth embodiment is an expression cassette, where the nucleotide sequence transcribed from the nucleotide sequence encoding the interfering RNA molecule includes at least 25 contiguous nucleotides which are partially or perfectly complementary to the entire length of SEQ ID NO:2.

An eighty-first embodiment is an expression cassette, where the nucleotide sequence encoding the interfering RNA molecule has at least 80% or at least 90% sequence similarity to the entire length of SEQ ID NO:1.

An eighty-second embodiment is an expression cassette, where the nucleotide sequence encoding the interfering RNA molecule includes the entire length of SEQ ID NO. 1.

An eighty-third embodiment is an expression cassette, where the nucleotide sequence encoding the interfering RNA molecule consists of the entire length of SEQ ID NO. 1.

An eighty-fourth embodiment is an expression cassette, where the expression cassette includes a nucleotide sequence with at least 70%, 80%, or 90% identity to the entire length of SEQ ID NO:7, or the complement thereof, or SEQ ID NO:8, or the complement thereof.

An eighty-fifth embodiment is an expression cassette, where the expression cassette consists of a nucleotide sequence with 100% identity to the entire length of SEQ ID NO:7, or the complement thereof, or SEQ ID NO:8, or the complement thereof.

An eighty-sixth embodiment is an expression cassette, where the expression cassette is stably integrated into a host organism's genome by transposition.

An eighty-seventh embodiment is an expression cassette, where the expression cassette is stably integrated in at least one site or at least three sites the host organism's genome.

An eighty-eighth embodiment is an expression cassette, where the expression cassette is integrated into one, two, three, four, five, six, seven, eight, nine, or ten sites of the host organism's genome.

An eighty-ninth embodiment is an expression cassette stably integrated into the genome of a host organism, where the host organism is a yeast cell.

A ninetieth embodiment is an expression cassette stably integrated into the genome of a host organism, where the host organism is Pichia pastoris, Saccharomyces cerevisiae, or Yarrowia lipolytica.

A ninety-first embodiment is an expression cassette, where the nucleotide sequence encoding the interfering RNA molecule is integrated at any one or more of genomic sites of Saccharomyces cerevisiae strain FL100 at chromosome IV (NC_001136 at position 543,705), chromosome IV (NC_001136 position 1,357,520), chromosome VIII (NC_001140 position 124,029), chromosome X (NC_001142 position 181,309), chromosome XI (NC_001143 position 300,654), or chromosome XII (NC_001144 position 213,991).

A ninety-second embodiment is a composition including the yeast cell or the expression cassette of any of the preceding embodiments.

A ninety-third embodiment is a composition including the yeast cell of any of the preceding embodiments, where the yeast cell is killed by heat and/or lyophilized.

A ninety-fourth embodiment is a composition further including a sugar bait.

A ninety-fifth embodiment is a composition further including an insecticide.

A ninety-sixth embodiment is a composition, where the composition is within a trap.

A ninety-seventh embodiment is a composition, where the composition is selectively insecticidal to a mosquito.

A ninety-eighth embodiment is a composition, which is selectively mosquitocidal, where the mosquito is a larval mosquito or an adult mosquito.

A ninety-ninth embodiment is a composition, which is selectively mosquitocidal to a species of Aedes, Anopheles, or Culex.

A hundredth embodiment is a composition, which is selectively mosquitocidal to Aedes aegypti, Aedes albopictus, Anopheles gambiae, Culex pipiens, or Culex quinquesfasciatus.

A hundred-and-first embodiment is a method for stably integrating an expression cassette including a nucleotide sequence encoding an interfering RNA molecule into the genome of a host organism with a transposase/transposon system, where the interfering RNA molecule inhibits the expression of a gene in a mosquito.

A hundred-and-second embodiment is a method for stably integrating an expression cassette including a nucleotide sequence encoding an interfering RNA molecule into the genome of a host organism, where the interfering RNA molecule is an RNA construct, a double stranded RNA (dsRNA), a small interfering RNA (siRNA), a short hairpin RNA (shRNA), or an anti-sense oligonucleotide.

A hundred-and-third embodiment is a method for stably integrating an expression cassette including a nucleotide sequence encoding an interfering RNA molecule into the genome of a host organism, where a nuclease system is used to generate a deletion mutation in an auxotrophic rescue gene in the host organism.

A hundred-and-fourth embodiment is a method for generating a deletion mutation in an auxotrophic rescue gene in the host organism, where the auxotrophic mutation inhibits the host organism's ability to produce histidine, leucine, tryptophan, uracil, or a combination thereof.

A hundred-and-fifth embodiment is a method for generating a deletion mutation in an auxotrophic rescue gene in the host organism, where the auxotrophic mutation includes ura3Δ0, leu2Δ0, his3Δ0, trp1Δ0, or a combination thereof.

A hundred-and-sixth embodiment is a method for generating a deletion mutation in an auxotrophic rescue gene in the host organism, where the auxotrophic mutations are ura3Δ0, leu2Δ0, his3Δ0, and trp1Δ0.

A hundred-and-sixth embodiment is a method for generating a deletion mutation in an auxotrophic rescue gene in the host organism with use of a nuclease system, where the nuclease system includes a dimeric C1051 endonuclease of at least about 95% sequence identity to SEQ ID:16.

A hundred-and-seventh embodiment is a method for generating a deletion mutation in an auxotrophic rescue gene in the host organism with use of a nuclease system, where the nuclease system includes a dimeric C1051 endonuclease of at least about 95% sequence identity to SEQ ID:16.

A hundred-and-eighth embodiment is a method for generating a deletion mutation in an auxotrophic rescue gene in the host organism with use of a nuclease system, where the nuclease system includes further includes two guide RNAs transcribed from gRNA nucleotide sequences having at least 90% identity to the entire length of SEQ ID NO:17 and SEQ ID NO:18; or SEQ ID NO:19 and SEQ ID NO:20; and where the gRNA nucleotide sequences are operably linked to an SNR52 promoter including a nucleotide sequence having about 90% identity to SEQ ID NO:13 and an SNR52 terminator including a nucleotide sequence having about 95% identity to SEQ ID NO:14.

A hundred-and-ninth embodiment is a method for method for stably integrating an expression cassette including a nucleotide sequence encoding an interfering RNA molecule into the genome of a host organism with a transposase/transposon system, where the transposase/transposon system includes a transposon vector and a transposase; and where the transposase integrates the transposon expression cassette into at least one integration site in the genome of the host organism.

A hundred-and-tenth embodiment is a method for method for stably integrating an expression cassette including a nucleotide sequence encoding an interfering RNA molecule into the genome of a host organism with a transposase/transposon system, where the transposase integrates the expression cassette into two, three, four, five, six, seven, eight, nine, or ten sites in the genome of the host organism.

A hundred-and-eleventh embodiment is a method for stably integrating an expression cassette including a nucleotide sequence encoding an interfering RNA molecule into the genome of a host organism with a transposase/transposon system, where the expression cassette further includes an auxotrophic rescue promoter and an auxotrophic rescue gene.

A hundred-and-twelfth embodiment is a method for stably integrating an expression cassette including an auxotrophic rescue promoter and an auxotrophic rescue gene, where the auxotrophic rescue promoter is flanked by a 5′ inverted terminal repeat and the nucleotide sequence encoding the interfering RNA molecule is flanked by a 3′ inverted terminal repeat.

A hundred-and-thirteenth embodiment is a method for stably integrating an expression cassette including a nucleotide sequence encoding an interfering RNA molecule into the genome of a host organism with a transposase/transposon system, where the nucleotide sequence encoding the interfering RNA molecule is operably linked to a glyceraldehyde-3-phosphate dehydrogenase GAP promoter, where the GAP promoter separates the nucleotide sequence encoding the interfering RNA molecule from the 3′ inverted terminal repeat sequence.

A hundred-and-fourteenth embodiment is a method for stably integrating an expression cassette including a nucleotide sequence encoding an interfering RNA molecule into the genome of a host organism with a transposase/transposon system, where the nucleotide sequence encoding the interfering RNA molecule is flanked on the 3′ end by a CYC1 terminator, where the CYC1 terminator separates the nucleotide sequence encoding the interfering RNA molecule from the 5′ inverted terminal repeat sequence.

A hundred-and-fifteenth embodiment is a method for stably integrating an expression cassette including a GAP promoter and a CYC1 terminator into the genome of a host organism, where the GAP promoter includes a nucleotide sequence with at least 85% sequence identity to SEQ ID NO:11 and the CYC1 terminator includes a nucleotide sequence with at least 85% sequence identity to SEQ ID NO:12.

A hundred-and-sixteenth embodiment is a method for method for stably integrating an expression cassette including a nucleotide sequence encoding an interfering RNA molecule into the genome of a host organism with a transposase/transposon system, where the expression cassette further includes an auxotrophic rescue promoter operably linked to an auxotrophic rescue gene, and where the auxotrophic rescue promoter is flanked by the 5′ inverted terminal repeat sequence.

A hundred-and-seventeenth embodiment is a method for method for stably integrating an expression cassette including an auxotrophic rescue promoter operably linked to an auxotrophic rescue gene into the genome of a host organism, where the auxotrophic rescue promoter is a minimal auxotrophic rescue promoter.

A hundred-and-eighteenth embodiment is a method for method for stably integrating an expression cassette including a minimal auxotrophic rescue promoter into the genome of a host organism, where the minimal auxotrophic rescue promoter includes a nucleotide sequence with at least 70% identity to the entire length of SEQ ID NO:3.

A hundred-and-nineteenth embodiment is a method for method for stably integrating an expression cassette including a minimal auxotrophic rescue promoter into the genome of a host organism, where the minimal auxotrophic rescue promoter includes a nucleotide sequence with 100% identity to the entire length of SEQ ID NO:3.

A hundred-and-twentieth embodiment is a method for stably integrating an expression cassette into the genome of a host organism with a transposon/transposase system, where the expression cassette further includes an insulator sequence.

A hundred-and-twenty-first embodiment is method for stably integrating an expression cassette including an insulator sequence into the genome of a host organism with a transposon/transposase system, where the insulator sequence separates the 5′ end of the nucleotide sequence encoding the interfering RNA molecule from the 3′ inverted terminal repeat sequence.

A hundred-and-twenty-second embodiment is method for stably integrating an expression cassette including an insulator sequence into the genome of a host organism with a transposon/transposase system, where the insulator sequence separates the 3′ end of the nucleotide sequence encoding the interfering RNA molecule from the 5′ inverted terminal repeat sequence.

A hundred-and-twenty-third embodiment is method for stably integrating an expression cassette including an insulator sequence into the genome of a host organism with a transposon/transposase system, where the insulator sequence has 70% identity to the entire length of SEQ ID NO:6.

A hundred-and-twenty-fourth embodiment is method for stably integrating an expression cassette including an insulator sequence into the genome of a host organism with a transposon/transposase system, where the insulator sequence has 100% identity to the entire length of SEQ ID NO:6.

A hundred-and-twenty-fifth embodiment is method for stably integrating an expression cassette including a 5′ inverted terminal repeat sequence into the genome of a host organism with a transposon/transposase system, where the 5′ inverted terminal repeat sequence has at least about 70% identity to the entire length of SEQ ID NO:5.

A hundred-and-twenty-sixth embodiment is method for stably integrating an expression cassette including a 5′ inverted terminal repeat sequence into the genome of a host organism with a transposon/transposase system, where the 5′ inverted terminal repeat sequence has 100% identity to the entire length of SEQ ID NO:5.

A hundred-and-twenty-seventh embodiment is method for stably integrating an expression cassette including a 5′ inverted terminal repeat sequence into the genome of a host organism with a transposon/transposase system, where the 5′ inverted terminal repeat sequence consists of the entire length of SEQ ID NO:5.

A hundred-and-twenty-eighth embodiment is method for stably integrating an expression cassette including a 3′ inverted terminal repeat sequence into the genome of a host organism with a transposon/transposase system, where the 3′ inverted terminal repeat sequence has at least about 70% identity to the entire length of SEQ ID NO:4.

A hundred-and-twenty-ninth embodiment is method for stably integrating an expression cassette including a 3′ inverted terminal repeat sequence into the genome of a host organism with a transposon/transposase system, where the 3′ inverted terminal repeat sequence has 100% identity to the entire length of SEQ ID NO:4.

A hundred-and-thirtieth embodiment is method for stably integrating an expression cassette including a 3′ inverted terminal repeat sequence into the genome of a host organism with a transposon/transposase system, where the 3′ inverted terminal repeat sequence consists of the entire length of SEQ ID NO:4.

A hundred-and-thirty-first embodiment is method for stably integrating an expression cassette into the genome of a host organism with a transposon/transposase system, where the expression cassette includes at least one, at least three, or at least five copies of the nucleotide sequence encoding the interfering RNA molecule.

A hundred-and-thirty-second embodiment is a method for stably integrating an expression cassette including a nucleotide sequence encoding an interfering RNA molecule into the genome of a host organism with a transposase/transposon system, where the interfering RNA molecule is capable of inhibiting the expression of Shaker in a mosquito.

A hundred-and-thirty-third embodiment is a method for stably integrating an expression cassette including a nucleotide sequence encoding an interfering RNA molecule into the genome of a host organism with a transposase/transposon system, where a nucleotide sequence transcribed from the nucleotide sequence encoding the interfering RNA molecule includes at least 25 contiguous nucleotides which are partially or perfectly complementary to a sequence including at least 80%, 84%, 88%, 92%, 96%, or 100% identity to the entire length of SEQ ID NO:2.

A hundred-and-thirty-fourth embodiment is a method for stably integrating an expression cassette including a nucleotide sequence encoding an interfering RNA molecule into the genome of a host organism with a transposase/transposon system, where a nucleotide sequence transcribed from the nucleotide sequence encoding the interfering RNA molecule includes at least 25 contiguous nucleotides which are partially or perfectly complementary to the entire length of SEQ ID NO:2.

A hundred-and-thirty-fifth embodiment is a method for stably integrating an expression cassette including a nucleotide sequence encoding an interfering RNA molecule into the genome of a host organism with a transposase/transposon system, where the nucleotide sequence encoding the interfering RNA molecule has at least 80% or at least 90% sequence similarity to the entire length of SEQ ID NO. 1.

A hundred-and-thirty-sixth embodiment is a method for stably integrating an expression cassette including a nucleotide sequence encoding an interfering RNA molecule into the genome of a host organism with a transposase/transposon system, where the nucleotide sequence encoding the interfering RNA molecule has 100% identity to the entire length of SEQ ID NO. 1.

A hundred-and-thirty-seventh embodiment is a method for stably integrating an expression cassette including a nucleotide sequence encoding an interfering RNA molecule into the genome of a host organism with a transposase/transposon system, where the nucleotide sequence encoding the interfering RNA molecule consists of the entire length of SEQ ID NO. 1.

A hundred-and-thirty-eighth embodiment is a method for stably integrating an expression cassette into the genome of a host organism with a transposase/transposon system, where the expression cassette includes a nucleotide sequence with at least 70%, 80%, or 90% identity to the entire length of SEQ ID NO:7, or the complement thereof, or SEQ ID NO:8, or the complement thereof.

A hundred-and-thirty-ninth embodiment is a method for stably integrating an expression cassette into the genome of a host organism with a transposase/transposon system, where the expression cassette consists of a nucleotide sequence with 100% identity to the entire length of SEQ ID NO:7, or the complement thereof, or SEQ ID NO:8, or the complement thereof.

A hundred-and-fortieth embodiment is a method for stably integrating an expression cassette into the genome of a host organism with a transposase/transposon system, where the transposase includes a nucleotide sequence with at least about 70% identity to SEQ ID NO:10.

A hundred-and-forty-first embodiment is a method for stably integrating an expression cassette into the genome of a host organism with a transposase/transposon system, where the transposase includes a nucleotide sequence with 100% identity to SEQ ID NO:10.

A hundred-and-forty-second embodiment is a method for controlling a mosquito population, including feeding a mosquito with the yeast cell of any one of claims 1-52, the expression cassette of any one of claims 54-91, or the composition of any one of claims 92-101, thereby controlling the mosquito population.

A hundred-and-forty-third embodiment is a method for controlling a mosquito population, where the mosquito is a larval mosquito or an adult mosquito.

A hundred-and-forty-fourth embodiment is a method for controlling a mosquito population, where the mosquito is a species of Aedes, Anopheles, or Culex.

A hundred-and-forty-fifth embodiment is a method for controlling a mosquito population, where the mosquito is Aedes aegypti, Anopheles gambiae, or Culex quinquefasciatus.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A. Schematic of S. cerevisiae engineering depicting precise cutting by dimeric Cas-CLOVER and two gRNAs targeting genes that encode essential amino acids required for growth.

FIG. 1B. Schematic of S. cerevisiae engineering depicting use of Super PiggyBac transposase/transposon system to integrate biopesticide cargo along with a selectable auxotrophic marker (PNut=nutritional promoter; N.gene=nutrition gene) randomly at ‘TTAA’ sites, which allows ease of detection and positive genetic integration.

FIG. 2. Bar graph showing comparative expression levels of an shRNA (Sh.463) targeting the mosquito Shaker gene in Cas-CLOVER/piggyBac-synthesized yeast strains relative to initial piggyBac integration strain DMT9-51.1.

FIG. 3. Bar graph showing down-selected and auxotrophy-restored Cas-CLOVER/piggyBac-synthesized yeast strains expressing high levels of an shRNA (Sh.463) targeting the mosquito Shaker gene relative to the initial piggyBac integration strain DMT4-51.1R #1.

FIG. 4. Chromosome map showing the genomic integration site of DMT9-56.10R #3.

FIGS. 5A, 5B, 5C, 5D, and 5E. Chromosome map showing the genomic integration site of DMT9-52.2R #3: FIG. 5A-5D show stable integration positions of an expression cassette encoding insecticidal shRNA Sh.463.

FIG. 6. Bar graph depicting larvicidal activity of Cas-CLOVER/piggyBac-synthesized yeast strains expressing shRNA targeting the mosquito Shaker gene.

FIG. 7. Bar graph showing adulticidal activity of Cas-CLOVER/piggyBac-synthesized yeast strains expressing shRNA targeting the mosquito Shaker gene.

FIG. 8. Line graph depicting growth curves (OD600) and shRNA (Sh.463) expression of DMT9-56.10R #3 in two different high cell density fermentation medias, HCD and DFM, with dashed lines representing the shRNA expression and solid lines representing the optical density.

### DETAILED DESCRIPTION

The utility of biopesticides that produce interfering RNA molecules has been demonstrated. However, improvements are necessary to effectively address the burden of mosquito-borne illnesses. For example, improvements to methods of engineering the host organism to produce the biopesticide, production of the biopesticide at an industrial scale, and methods of selectively delivering the biopesticide to the target organism could provide superior features and advantages.

In one example, an interfering RNA biopesticide targeting the mosquito gene Shaker, which encodes an evolutionarily conserved voltage-gated potassium channel subunit, has been shown to be required for mosquito survival (Mysore et al., PLoS Negl Trop Dis. 2020 July; 14(7): e0008479). Delivery of the interfering RNA molecule Sh.463 to Aedes aegypti adult mosquitoes in the form of siRNA that was injected or provided as an attractive toxic sugar bait (ATSB) led to silencing of Shaker that resulted in severe neural and behavioral defects and high levels of adult mortality. Likewise, when Sh.463 was provided to A. aegypti larvae in the form of short hairpin RNA (shRNA) expressed in Saccharomyces cerevisiae, i.e., an exemplary interfering RNA biopesticide as referred to herein, and formulated as a dried inactivated yeast tablet, the biopesticide induced neural defects, larval and adult death.

Additionally, the selectivity of Sh.463 in various mosquito populations was demonstrated. The gene target or Sh.463 lacks a known target site in humans and other non-target organisms, e.g., non-target arthropods. However, the target site is conserved in different mosquito species, e.g, Aedes spp., Anopheles spp., and Culex spp., as shown in Table 1, which is reproduced from Mysore et al., PLoS Negl Trop Dis. 2020 July; 14(7): e0008479.

TABLE 1
Evaluation of Sh.463 Target Site Conservation

| Mosquito Species | | |
|---|---|---|
| or Taxonomic | Identical | |
| Group/Taxonomic ID | Match? | Match Location |
| Aedes aegypti | Yes | AAEL000242 |
| Aedes albopictus | Yes | AALF022329 |
| Anopheles albimanus | Yes | AALB006583 |
| Anopheles arabiensis | Yes | AARA012058 |
| Anopheles christyi | Yes | KB689759 (s) |
| Anopheles culicifacies | Yes | ACUA008770 |
| Anopheles darlingi | Yes | scaffold_1546 (s) |
| Anopheles dirus | Yes | KB672491 (s) |
| Anopheles epiroticus | Yes | AEPI010172 |
| Anopheles farauti | Yes | AFAF011906 |
| Anopheles freeborni | Yes | SRS008481_104582 |
| Anopheles funestes | Yes | AFUN000256 |
| Anopheles gambiae | Yes | AGAP000254 |
| Anopheles maculatus | Yes | AMAM001333 |
| Anopheles melas | Yes | AMEC013303 |
| Anopheles merus | Yes | AXCQ02011250 (s) |
| Anopheles minimus | Yes | AMIN005805, AMIN009888 |
| Anopheles punctulatus | Yes | JXXA01008934.1 (s) |
| Anopheles quadrimaculatus | Yes | GBTE01015309.1 |
| Anopheles stephensi | Yes | ASTE005450 ASTEI01122 |
| Culex quinquefasciatus | Yes | supercont3.226 (s) |
| Amphibians/8292 | No | N/A |
| Arthropoda/6656 | No | N/A |
| (other than mosquitoes) | | |
| Birds/8782 | No | N/A |
| Coleoptera/7041 | No | N/A |
| Crustacea/6657 | No | N/A |
| Diptera/7147 | No | N/A |
| (other than mosquitoes) | | |
| Fish/7898 | No | N/A |
| Fungi/4751 | No | N/A |
| Human/9606 | No | N/A |
| Mammals/40674 | No | N/A |
| Plants/3193 | No | N/A |
| Reptiles/8504 | No | N/A |

Previous methods of engineering a host organism to produce the interfering RNA molecule involved ligating the nucleotide sequence encoding shRNA.463 into non-integrating and integrating shuttle vectors. See, e.g., Haipairai et al., Sci Rep. 2017; 7: 13223 and Mysore et al., PLoS Negl Trop Dis. 2020 July; 14(7): e0008479. Disclosed herein are improved methods of engineering an interfering RNA biopesticide, which have the exemplary advantages of efficient and stable integration into the genome of a host organism, enhanced expression of the interfering RNA molecule, and feasibility of industrial scale up. In one aspect, described herein is the production of interfering RNA biopesticides for control of disease vector mosquitoes using the Cas-CLOVER system in conjunction with a transposon system, such as the piggyBac transposon and the Super piggyBac transposase. Herein, a “transposon system” may be referred to interchangeably as a “transposon/transposase system” or a “transposase/transposon system.”

While Saccharomyces cerevisiae is a natural attractant to mosquitoes, it lacks the necessary machinery to process the interfering RNA molecules disclosed herein. The exemplary yeast host organism is amenable to gene editing through Cas-CLOVER, which can enable production of various molecules. However, microbial genomes are generally non-redundant compared to plant and mammalian genomes. This feature presents a technical challenge to nuclease and transposon-mediated gene editing, as off-target cutting or random integration on an essential gene can result in cell death, and thereby reduce the likelihood of survivable clones.

Cas-CLOVER, like the original CRISPR/Cas9 system, is an RNA guided system. Although the Cas-CLOVER system maintains the simplicity of the original Cas9 system, unlike Cas9, it uses a dimeric nuclease called Clo051 to edit the genome. The Clo51 subunit is an obligate dimer, meaning that it must dimerize in order to perform cleaving activity. Clo051, is associated with reduced off-target nuclease activity relative to CRISPR, such as higher fidelity, larger deletions, and more efficient knock-ins.

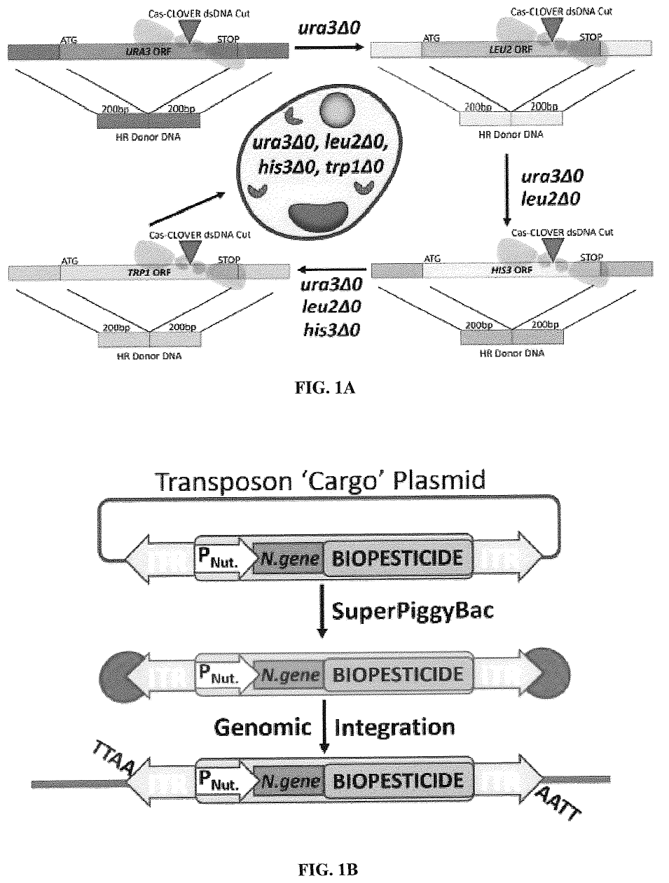

Cas-CLOVER was used to systematically create yeast strains bearing multiple gene deletions that are easily rescued by selectable markers (ura3, leu2, his3, trp1), thereby creating a versatile bioprocessing platform. All auxotrophic marker gene knockouts were full ORF deletions facilitated by homology directed repair (post-Cas-CLOVER cutting) with donor PCR fragments that encompass 200 bp upstream of the translation start-site and 200 bp downstream of the stop codon (FIG. 1A). Utilizing the resulting auxotrophic engineered yeast strains in conjunction with Super PiggyBac transposase/transposon technology provided efficient detection and selection of positive integrated cargo (FIG. 1B).

In addition to the versatile bioprocessing platform for transposase integration, S. cerevisiae lack components of the RNA interference (RNAi) pathway, enabling this organism to become a cell biofactory and delivery system of interfering RNA molecules for control of disease vector mosquitoes, which kill hundreds of thousands of people across the globe each year. See, e.g., Duman-Scheel. Current Drug Targets. 20 (9), 942-952 and Mysore et al., Insect genomics: Methods and protocols. 1858; 213-231. Humana Press.

The term “siRNA,” or “small interfering RNA,” refers to short interfering RNA or silencing RNA, which are short double-stranded RNA molecules of <30 base pairs in length, for example, about 19-30 base pairs in length that operate through the RNAi pathway. Each siRNA is unwound into two single-stranded RNAs (ssRNAs), one of which is incorporated into the RNA-induced silencing complex (RISC) leading to post-transcriptional gene silencing. siRNAs can be generated in several ways. In some cases, long dsRNA is introduced to a cell, either by a virus, endogenous RNA expression (i.e., microRNA), or exogenously delivered dsRNA. The enzyme DICER cleaves the long duplex RNAs into siRNAs. In some preferred embodiments, the siRNA is about 25 bp in length. While use of longer (300-400 bp) double stranded RNA (dsRNA) molecules is one approach for producing interfering RNA, the short length (21-25 bp) of custom small interfering RNAs (siRNAs) facilitates the design of highly specific interfering RNA.

The terms “short hairpin RNA” and “small hairpin RNA” are encompassed by the term “shRNA.” shRNAs are artificial RNAs having a secondary structure such that a portion of the RNA strand forms a hairpin loop. shRNA can be transcribed under the control of RNA Pol-II or Pol-III promoters, and folds into a structure resembling a siRNA duplex. shRNAs are then processed by DICER into siRNAs.

The term “dsRNA” (double stranded RNA) refers to long double-stranded RNA molecules that are cleaved by the enzyme DICER into short double-stranded fragments of about 20-25 nucleotide siRNAs.

RNA interference (RNAi) or Post-Transcriptional Gene Silencing (PTGS) refers to the biological process in which RNA molecules interfere or inhibit the expression of specific genes with complementary nucleotide sequences to the interfering RNA (gene-specific suppression of gene expression). RNAi results in the degradation of mRNA after transcription, resulting in reduced translation and protein expression.

RNA interference techniques employ genetic constructs that encode interfering RNA molecules, such as dsRNA and shRNA. Typically, the RNA constructs comprise sense and anti-sense sequences which are placed in regions flanking an intron sequence in proper splicing orientation with donor and acceptor splicing sites. Alternatively, spacer sequences of various lengths can be employed to separate self-complementary regions of sequence in the construct. During processing of the gene construct transcript, intron sequences can be spliced-out, allowing sense and anti-sense sequences, as well as splice junction sequences, to bind forming double-stranded RNA. Alternatively, where secondary structure inhibits splicing machinery, the intron sequences are not spliced out and the dsRNA is supplied as a hairpin structure. When the dsRNA is expressed in a cell, ribonucleases bind to and cleave the double-stranded RNA, initiating a cascade of events leading to degradation of the target mRNA molecules, and thereby silencing such target genes. The phenomenon of RNA interference using shRNA is described in Sheng et al., Front Bioeng Biotechnol. 2020 Aug. 7; 8:940 and generally in Bass, Nature 411: 428-29 (2001); Elbahir et al., Nature 411: 494-98 (2001); and Fire et al., Nature 391: 806-11 (1998); and WO 01/75164, where methods of making interfering RNA also are discussed.

The interfering RNA can hybridize with the full-length mRNA encoded by the target gene or hybridize to a fragment of the target RNA or DNA (the target sequence). For example, to reduce expression of a target gene in a mosquito by RNA interference, an expression cassette encoding an interfering RNA molecule having the sequence of an mRNA transcribed from the target gene, or a substantially identical sequence (including those engineered not to translate the protein), or fragment thereof, is stably integrated into the genome of a host organism, such as a yeast cell.

Stable integration refers to sustaining long-term expression of a transgene by integrating foreign DNA into the host nuclear genome. Accordingly, foreign DNA, or the transgene, is integrated into the genome, replicated alongside genomic DNA, and passed down to progeny. In contrast, nucleic acids are not integrated into the host cell genome in transient transfection. In transient systems, foreign DNA is unable to replicate independently from the host's DNA and may persist only for a few days. See, e.g., Fus-Kajawa, Front Bioeng Biotechnol. 2021 Jul. 20; 9:701031.

While stable integration was achieved for parental strains, see, e.g., US20220346380Δ1, of the interfering RNA biopesticides disclosed herein, e.g., DMT9-52.2R #3 and DMT9-56.10R #3, the parental strains did not grow at sufficient rates for scaled fermentation. Parental strains also included a galactose-inducible promoter, which would have driven up costs for scaled yeast production. In comparison to the parental strains, the strains disclosed herein express disclosed shRNA at substantially higher level, thereby reducing the dose of yeast required to kill mosquitoes. This feature is anticipated to reduce the costs of commercial production and deployment.

The resulting genetically modified host organism can then be fed to the mosquito to determine its ability to inhibit expression of the target gene, which results in reduced fitness and/or survival of the mosquito. Although the sequence of the interfering RNA used for RNA interference need not be completely identical to mRNA transcribed from the target sequence of the target gene, it is typically substantially identical, e.g., at least 70%, 80%, 90%, 95%, 98%, or more identical to mRNA transcribed from the target sequence. It is known in the art that dsRNA molecules that are not perfectly complementary to mRNA transcribed from the target sequence (for example, having only 95% identity to mRNA transcribed from the target sequence) are effective to control insect pests (see, for example, Narva et al., U.S. Pat. No. 9,012,722).

Target genes can be selected based on a number of criteria, including gene essentiality, expression level, and divergence from related species sequences. Exemplary target genes include Shaker and orthologs thereof, e.g., Shaker-like genes, which encode a voltage-gated potassium channel protein. Exemplary target organisms having the target gene or an ortholog thereof are shown in Table 2. The exemplary target genes and organisms were retrieved from NCBI's BLAST (https://blast.ncbi.nlm.nih.gov/Blast.cgi) by querying SEQ ID NO. 2.

TABLE 2
Exemplary Target Genes and Associated Gene IDs

| Description | Gene ID |
|---|---|
| Anopheles nili potassium voltage-gated | LOC128728591 |
| channel protein Shaker | |
| Anopheles marshallii potassium voltage-gated | LOC128706889 |
| channel protein Shaker | |
| Anopheles moucheti potassium voltage-gated | LOC128307365 |
| channel protein Shaker-like | |
| Anopheles cruzii potassium voltage-gated | LOC128273193 |
| channel protein Shaker | |
| Anopheles aquasalis potassium voltage-gated | LOC126572542 |
| channel protein Shaker | |
| Anopheles maculipalpis potassium voltage- | LOC126568426 |
| gated channel protein Shaker | |
| Anopheles darlingi potassium voltage-gated | LOC125951850 |
| channel protein Shaker | |
| Anopheles coluzzii potassium voltage-gated | LOC120958335 |
| channel protein Shaker | |
| Anopheles funestus potassium voltage-gated | LOC125766229 |
| channel protein Shaker | |
| Anopheles merus potassium voltage-gated | LOC121587667 |
| channel protein Shaker | |
| Anopheles arabiensis potassium voltage-gated | LOC120906401 |
| channel protein Shaker | |
| Anopheles stephensi potassium voltage-gated | LOC118507132 |
| channel protein Shaker | |
| Anopheles albimanus potassium voltage-gated | LOC118464114 |
| channel protein Shaker-like | |
| Culex pipiens pallens potassium voltage-gated | LOC120415071 |
| channel protein Shaker | |
| Culex quinquefasciatus potassium voltage- | LOC6041679 |
| gated channel protein Shaker transcript | |

In some examples, inhibiting a target gene interferes with the production of an essential protein. Exemplary target gene Shaker encodes a structural component of a voltage-dependent potassium channel. Mutations in the Drosophila Shaker gene, which is conserved in mosquito species such as Anopheles, result in hyperexcitability near axon branch points due to improper repolarization of neurons. See, e.g, Salkof & Wyman, Nature. 1981; 293(5829):228-30 and Lichtinghagen et al., EMBO J. 1990; 9(13):4399-407. These neural defects manifest behaviorally in uncontrolled movements (Jan & Jan, J Physiol. 1997; 505 (Pt 2):267-82). For example, Drosophila Shaker mutants exhibit aberrant movements, such as leg shaking (hence the name phenotypically descriptive name Shaker (Trout & Kaplan, J Neurobiol. 1973; 4(6):495-512).

“Gene suppression” or “down-regulation of gene expression” or “inhibition or suppression of gene expression” are used interchangeably and refer to a measurable or observable reduction in gene expression or a complete abolition of detectable gene expression at the level of protein product (“gene silencing”), and/or mRNA product from the gene. In some embodiments, gene suppression results in gene silencing, referring to the ability of the interfering RNA to target mRNA for degradation, resulting in disrupted translation, which prevents protein expression. The ability of the interfering RNA to suppress or down-regulate at least one gene leads to the suppression or inhibition of a mosquito's growth or maturation or death of the mosquito larvae or adult. The downregulation or inhibition may occur at the translational or post-translational stage of expression of the gene of interest by promoting transcript turnover, cleavage, or disruption of translation.

Inhibition of target gene expression may be quantified by measuring either the endogenous target RNA or the protein produced by translation of the target RNA and the consequences of inhibition can be confirmed by examination of the outward properties of the cell or organism. Techniques for quantifying RNA and proteins are well known to one of ordinary skill in the art.

The term “gene” refers to a polynucleotide sequence that comprises control and coding sequences necessary for production of a polypeptide (protein). The polypeptide can be encoded by a full-length coding sequence or by any portion of the coding sequence. A gene includes regions preceding and following the coding region (leader and trailer) as well as intervening sequences (introns) between individual coding segments (exons). The leader, the trailer as well as the introns include regulatory elements that are necessary during the transcription and the translation of a gene (e.g., promoters, enhancers, etc.). A gene may be an uninterrupted coding sequence or may include one or more introns contained between splice junctions. As used herein, a gene may include variants of the gene, which include, but are not limited to, modifications such as mutations, insertions, deletions, or substitutions of one or more nucleotides. A “target gene” is the gene targeted for down-regulation or suppression by the interfering RNA of the present technology. A “gene product” can refer to either the mRNA or protein expressed from a particular gene.

The terms “nucleic acid,” “polynucleotide,” and “oligonucleotide” refer to a single or double-stranded polymer of deoxyribonucleotide or ribonucleotide bases read from the 5′ to the 3′ end. The monomer is typically referred to as a nucleotide. Nucleic acids can include modified nucleotides that permit correct read through by a polymerase and do not significantly alter expression of a polypeptide encoded by that nucleic acid.

The phrase “nucleic acid sequence encoding” refers to a nucleic acid, such as DNA, which is the template for transcription of a specific RNA molecule, e.g., a shRNA, a dsRNA, or an mRNA that is translated into a protein. The nucleic acid sequences include both the full-length nucleic acid sequences as well as non-full-length sequences derived from the full-length sequences. A coding sequence can include degenerate codons (relative to the native sequence) or sequences that provide codon preference in a specific host cell.

The term “promoter” refers to regions or sequence located upstream and/or downstream from the start of transcription and which are involved in recognition and binding of RNA polymerase and other proteins to initiate transcription. A “yeast promoter” is a promoter capable of initiating transcription in yeast cells. A yeast promoter can be a nucleic acid sequence originally isolated from a yeast, but promoters not initially isolated from a yeast are also considered “yeast promoters” for the purposes of this disclosure.

An “expression cassette” refers to a nucleic acid construct, which when introduced into a host cell (e.g., a yeast cell), results in transcription of an RNA molecule (e.g., dsRNA or mRNA). An expression cassette typically includes a sequence to be expressed, and sequences necessary for expression of the sequence to be expressed, such as a promoter operably linked to the sequence. Generally, an expression cassette is inserted into an expression vector to be introduced into a host cell.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity can be partial, in which only some of the nucleic acids match according to base pairing, or complete, such as fully complementary or perfectly complementary, where all the nucleic acids match according to base pairing.

The terms “protein,” “peptide,” and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified.

The terms “identical” or percent “identity,” in the context of two or more nucleic acids or proteins of the invention, refer to two or more sequences or subsequences that are the same or have a specified percentage of nucleotides or amino acids that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST/. For example, the sequence of a dsRNA of the invention can be compared using the above techniques to the sequence of a target gene in a mosquito, taking into account the presence of uracil in the dsRNA and thymidine in the DNA. Sequences that have at least about 90% sequence identity using the methods described above are said to be “substantially identical.” This definition also refers to, and can be applied to, the complement of a test sequence. The definition also includes sequences that have deletions and/or additions, as well as those that have substitutions. Optimal alignment of such sequences can be carried out by any of the publicly available algorithms or programs for determining sequence identity and alignment, e.g., BLAST.

In some embodiments, the reduction, inhibition, or suppression of expression of the target gene results in life cycle disruptions, such as reduced viability, growth, development or reproduction of the targeted mosquito, including larvae and adult forms. Such assessments are within the grasp of one of skill in the art and described in, e.g., Mysore et al., PLoS Negl Trop Dis., 14(7): e0008479.

Other methods of confirming downregulation of the gene expression are known in the art, and include, but are not limited to, measurement of mRNA or protein expression using molecular techniques such as RNA solution hybridization, nuclease protection, Northern hybridization, reverse transcription, gene expression monitoring with a microarray, antibody binding, enzyme-linked immunosorbent assay (ELISA), Western blotting, radioimmunoassay (MA), other immunoassays, or fluorescence-activated cell analysis (FACS) and the like.

### Methods of Engineering Yeasts

In some aspects, provided are methods for stably integrating a polynucleotide sequence encoding an interfering RNA molecule into the genome of a host organism comprising use of a nuclease system and a transposase system, where the interfering RNA molecule inhibits the expression of a gene in a mosquito. Accordingly, disclosed are methods of engineering a host organism to express an interfering RNA molecule, where the interfering RNA molecule is capable of suppressing gene expression in a mosquito. Herein, such engineered host organism may be referred to as a “biopesticide” or an “interfering RNA biopesticide.”

In some embodiments, disclosed methods comprise integrating, such as stably integrating, an expression cassette comprising a nucleotide sequence encoding an interfering RNA molecule into the genome of a host organism. In some embodiments, integrating the expression cassette into the host organism genome comprises use of a nuclease system and a transposon system. In some embodiments, the interfering RNA molecule inhibits the expression of a gene in a mosquito.

In some embodiments, the nuclease system is used to generate a mutation in a gene in the host organism, such as a selectable marker gene. In some embodiments, the mutation is a deletion mutation. In some embodiments, the mutation is a partial or a full deletion of the open reading frame of the selectable marker gene. In some embodiments, the mutation is a full deletion of the open reading frame of the selectable marker gene. In some embodiments, the open reading frame deletion results from homology directed repair, such as homology directed repair initiated by nuclease activity.

In some embodiments, the selectable marker gene is a dominant marker gene, an auxotrophic rescue gene, or a combination thereof. In some embodiments, the dominant marker gene is KanMX, ble, Sh ble, hph, Cat, CUP1, SFA1, dehH1, PDR3-9, AURI-C, nat, CYH2, pat, ARO4-, OFP, SMR1, FZFI-4, DsdA, orthologs thereof, or a combination thereof.

In some embodiments, the selectable marker gene is an auxotrophic rescue gene. Herein, an “auxotrophic rescue gene” may be interchangeably referred to as an “auxotrophic marker gene.” In some embodiments, the auxotrophic rescue gene is URA3, KIURA3, CaURA3, HIS3, HIS5, LEU2, KILEU2, LYS2, TRP1, ADE1, ADE2, MET15, orthologs thereof, or a combination thereof. In some embodiments, the auxotrophic rescue gene is ade1-14, ade1-101, ade2-1, ade2-101, ade2 Δ::hisG, lys2Δ0, met15 Δ 0, ura3Δ0, ade2-BgIII, can1-100, his3Δ200, his3Δ0, his3Δ1, his3-11, his3-15, his3 Δ, leu2Δ1, leu2Δ0, leu2-3, leu2-112, leu2Δ, ys2-801 lys2Δ202, lys2Δ, trp1Δ0, trp1Δ1, trp1Δ63, trp1-1, trp1Δ, trp1-289, trp5Δ, ura3-52, ura3-1, ura3Δ, ura3Δ0, ura4delta, or a combination thereof. In some embodiments, the mutation generates an auxotrophic mutant.

In some embodiments, the nuclease system is used to generate an auxotrophic mutation in a microbial cell, such as a yeast cell. In some embodiments, the nuclease system is used to generate an auxotrophic mutant or a multiply auxotrophic mutant. In some embodiments, the nuclease system is used to generate a histidine auxotroph, a leucine auxotroph, a tryptophan auxotroph, or a uracil auxotroph. In some embodiments, the nuclease system is used to generate a multiply auxotrophic microbial cell, such as a yeast cell, having any two or more of histidine auxotrophy, leucine auxotrophy, tryptophan auxotrophy, and uracil auxotrophy. In some embodiments, the nuclease system is used to generate a multiply auxotrophic microbial cell, such as a yeast cell, having uracil auxotrophy and leucine auxotrophy. In some embodiments, the nuclease system is used to generate a multiply auxotrophic microbial cell, such as a yeast cell, having histidine auxotrophy and tryptophan auxotrophy.

In some embodiments, the auxotrophic mutation results from full open reading frame deletion of an auxotrophic rescue gene. In some embodiments, the full open reading frame deletion results from homology directed repair, such as homology directed repair initiated by nuclease activity. In some embodiments, the nuclease is Clo51, such as Clo51 in the Cas-CLOVER system.

In some embodiments, the auxotrophic mutation comprises ura3Δ0, leu2Δ0, his3Δ0, trp1Δ0, or a combination thereof. In some embodiments, the auxotrophic mutant comprises the auxotrophic mutations ura3Δ0 and leu2Δ0. In some embodiments, the auxotrophic mutant comprises the auxotrophic mutations ura3Δ0, leu2Δ0, his3Δ0, and trp10.

Deletion and restoration of various selectable markers may be leveraged to determine the identity of a transformant, such as the stable integration of a polynucleotide sequence or expression cassette as disclosed herein in a host organism, such as a yeast cell. For example, prototrophic markers, C/N-source related markers, resistance markers, and manipulation thereof are described by, e.g., Siewers, Methods Mol Biol. 2014; 1152:3-15.

In other examples, alternative gene editing nuclease systems may be used to generate an auxotrophic yeast strain that is suitable for use in accordance with the disclosed methods. In additional examples, auxotrophic yeast strains may be provided for transposon-mediated integration of disclosed shRNA and expression cassettes in the production of interfering RNA biopesticides.

An auxotrophic mutant carries a mutation that renders it unable to synthesize an essential compound, such as a mutation in a gene that facilitates production of an amino acid or nucleotide biosynthesis. Auxotrophic strains are unable to grow unless the medium is supplemented with the necessary amino acid or nucleotide. With such strains, it is possible to select transformants by using plasmids that carry a wild-type copy of the mutated gene, provided that the reversion frequency of the mutation is lower than the transformation frequency. The latter condition is ensured by using non-reversible knockout mutations.

Auxotrophic selection markers, such as auxotrophic rescue genes, can complement the auxotrophic mutation and restore the growth of auxotrophic mutants. See, e.g, Ulfstedt et al., Front Plant Sci. 2017 Nov. 3; 8:1850. Auxotrophic rescue genes such as URA3, LEU2, or HIS3 are ubiquitous in yeast genetics, where they are used to select cells that have been successfully transformed with recombinant DNA. Auxotrophic markers and their application are further described by Yuan, PLoS ONE 2011; 6(10):e25830), Solis-Escalante et al., FEMS Yeast Res. 2013 January; 13(1): 126-139, and wiki.yeastgenome.org (“Commonly used auxotrophic markers”).

In some embodiments, disclosed methods comprise use of a nuclease system to generate an auxotrophic mutation or an auxotrophic mutant. In some embodiments, the nuclease system comprises at least one guide RNA (gRNA) and a nuclease, such as an endonuclease. In some embodiments the nuclease system comprises one gRNA and a nuclease. In some embodiments the nuclease system comprises at least two gRNAs and a nuclease. In some embodiments the nuclease system comprises two gRNAs and a nuclease. In some embodiments the nuclease is a dimeric nuclease. In some embodiments, the nuclease system comprises two guide RNAs and a dimeric nuclease. In some embodiments, the nuclease system is Cas-CLOVER.

In some embodiments, the nuclease system comprises a gRNA, such as an sgRNA, transcribed from a sequence having at least about 80%, or 90% identity to the entire length of SEQ ID NO:17, SEQ ID NO:18, or the complement thereof. In some embodiments, the nuclease system comprises a gRNA, such as an sgRNA, transcribed from a sequence having about 75%, 80%, 85%, 90%, 95%, 100% identity to the entire length of SEQ ID NO:17, SEQ ID NO:18, or the complement thereof. In some embodiments, the nuclease system comprises two gRNAs transcribed from the entire length of SEQ ID NO:17 and SEQ ID NO:18.

In some embodiments, the nuclease system comprises a gRNA, such as an sgRNA, transcribed from a sequence having at least about 80%, or 90% identity to the entire length of SEQ ID NO:19, SEQ ID NO:20, or the complement thereof. In some embodiments, the nuclease system comprises a gRNA, such as an sgRNA, transcribed from a sequence having about 75%, 80%, 85%, 90%, 95%, 100% identity to the entire length of SEQ ID NO:19, SEQ ID NO:20, or the complement thereof. In some embodiments, the nuclease system comprises two gRNAs transcribed from the entire length of SEQ ID NO:19 and SEQ ID NO:20.

In some embodiments, the nuclease system comprises a dimeric nuclease. In some embodiments, the dimeric nuclease has at least about 80%, 85%, 90%, or 95% sequence identity to the entire length of SEQ ID NO:16. In some embodiments, the dimeric nuclease has about 80%, 85%, 90%, 95%, 98%, 99%, or 100% sequence identity to the entire length of SEQ ID NO:16. In some embodiments, the dimeric nuclease is Clo051.

Cas-CLOVER is a dimeric gene editing system that exhibits greater precision, such as a lack of off-targets, relative to CRISPR-Cas9. While synthetic CRISPR employs a single guide RNA (sgRNA) together with CRISPR-associated proteins (Cas) to bind and cut target genomic sequences, Cas-CLOVER utilizes two gRNAs. Cas-CLOVER uses a catalytically inactive Cas fusion protein, or “dead Cas” (abbreviated as dCas), fused to the dimeric Clo051 endonuclease domain.

In the Cas-CLOVER system, two gRNAs can be designed to target genes of interest to create double-stranded breaks, a similar feature in other dimeric gene editing technologies, i.e. ZFN and TALEN. The Cas-CLOVER system requires a protospacer adjacent motif (PAM) sequence in each RNA. The two guide-RNAs should be designed in the PAMs-out orientation. A flexible spacer range between the two guides of 15-30 nucleotides enables the dual-complex to function. The flexibility of Cas-CLOVER's guide RNA design allows the user to target any gene of interest.

Dual synthetic gRNA/Cas-CLOVER complexes interact with the left and right complementary sequences within the targeted locus, which leads to dimerization of the Clo051 nuclease domains and cleavage of the targeted locus. Creation of a double-stranded break result in DNA repair mechanism activation and repair of the break via non-homologous end joining (NHEJ) or homologous recombination (HR). A variety of online open-source gRNA design tools are available to one of skill in the art and include CRISPR-MIT, E-CRISP, or CHOPCHOP, CRISPOR or ZiFit.

When using Cas-CLOVER in a plasmid-based editing system, the two guide-RNAs should be designed in the PAMs-out orientation. Specifically, when designing and creating the left guide, the sequence matches the bottom strand of the chosen guide sequence, reading 5′ to 3′ orientation toward the PAM site. This 5′ to 3′ sequence should be cloned into the vector between an appropriate promoter for the system and a guide scaffold. When designing and creating the right guide, the sequence should match the top strand, reading 5′ to 3′ toward a PAM site. The 5′ to 3′ sequence for this guide should also be cloned into the vector between an appropriate promoter and a guide scaffold. There are various ways to create guides for cloning into vectors, which include synthesized fragments or annealing together two single-stranded oligos to make a double stranded fragment. There are also several ways to clone the guides into vectors, which are evident to one of skill in the art. It is important to choose left and right guides that are separated by a spacer region of 15-30 nucleotides (Demeetra, “Designing Cas-CLOVER: A Dimeric RNA Guided Targeted Nuclease for Precision Gene-Editing,” User Guide. Cas-CLOVER for Gene Editing).

Exemplary dCas9-Clo051 fusion proteins (referred to in the art as “Cas-CLOVER” proteins), and polynucleotide sequences encoding said dCas9-Clo051 fusion proteins, are described in detail in U.S. Patent Publication No. 2022/0042038, the contents of which is incorporated herein by reference in its entirety. Gene editing compositions, including Cas-CLOVER, and methods of using these compositions for gene editing are described in detail in U.S. Patent Publication Nos. 2017/0107541, 2017/0114149, 2018/0187185 and US20160060610Δ1, the contents of each of which are incorporated herein by reference in their entireties.

In some embodiments, disclosed methods comprise integrating an expression cassette comprising a nucleotide sequence encoding an interfering RNA molecule into the genome of the host organism by transposition. Herein, transposition refers to a transposon-mediated method of integrating a transgene, including an expression cassette carrying the transgene, into the genome of a host organism.

In some embodiments, a transposon system, such as a is used to integrate an expression cassette comprising a nucleotide sequence encoding an interfering RNA molecule into the genome of a host organism. In some embodiments, the transposon system comprises a transposon vector and a transposase. In some embodiments, disclosed methods comprise integrating the expression cassette into at least one, two, three, four, five, six, seven, eight, nine, or ten sites in the genome of the host organism. In some embodiments, disclosed methods comprise integrating the expression cassette into one, two, three, four, five, six, seven, eight, nine, or ten sites in the genome of the host organism.

In some embodiments, disclosed methods comprise using the transposon system piggyBac, Super piggyBac, Sleeping Beauty, Hyperactive Sleeping Beauty, a helitron transposon system, a Tol2 transposon system, a TcBuster transposon system, including like, e.g., piggyBac-like, or mutant transposon systems thereof to integrate an expression cassette comprising a nucleotide sequence encoding an interfering RNA molecule into the genome of a host organism. In some embodiments, the nucleotide sequence encoding the transposase is operably linked to a constitutive promoter. In some embodiments, the nucleotide sequence encoding the transposase is operably linked to an ADH1 promoter or a derivative thereof, such as a truncated version.

In some embodiments, disclosed methods comprise integrating an expression cassette comprising an inverted terminal repeat sequence into the genome of a host organism by transposition, such as with use of a transposon system. In some embodiments, the transposon system comprises a transposon vector and a transposase. In some embodiments, the transposase is a piggyBac™ (PB) transposase, a piggyBac-like (PBL) transposase, a Super piggyBac™ (SPB) transposase polypeptide, a Sleeping Beauty transposase, a Hyperactive Sleeping Beauty (SB100X) transposase, a helitron transposase, a Tol2 transposase, a TcBuster transposase, or a mutant TcBuster transposase.

In some embodiments, disclosed methods comprise integrating an expression cassette comprising a 5′ inverted terminal repeat sequence and a 3′ inverted terminal repeat sequence into the genome of the host organism. In some embodiments, the inverted terminal repeat sequence is recognized by a piggyBac, Super piggyBac, or a piggyBac-like transposase. In some embodiments, the inverted terminal repeat sequence is recognized by a Sleeping Beauty transposase. In some embodiments, the inverted terminal repeat sequence is recognized by a Helraiser transposon. In some embodiments, the inverted terminal repeat sequence is recognized by a Tol2 transposon. In some embodiments, the inverted terminal repeat sequence is recognized by a TcBuster transposase.

In some embodiments, the 5′ inverted terminal repeat sequence has at least about 70%, 75%, 80%, 85%, 90%, 95%, 99%, or 100% identity to the entire length of SEQ ID NO:5. In some embodiments, the 5′ inverted terminal repeat sequence has about 70%, 75%, 80%, 85%, 90%, 95%, 99%, or 100% identity to the entire length of SEQ ID NO:5. In some embodiments, the 5′ inverted terminal repeat sequence has 100% identity to the entire length of SEQ ID NO:5. In some embodiments, the 5′ inverted terminal repeat sequence consists of to the entire length of SEQ ID NO:5.

In some embodiments, the 3′ inverted terminal repeat sequence has at least about 70%, 75%, 80%, 85%, 90%, 95%, 99%, or 100% identity to the entire length of SEQ ID NO:4. In some embodiments, the 3′ inverted terminal repeat sequence has about 70%, 75%, 80%, 85%, 90%, 95%, 99%, or 100% identity to the entire length of SEQ ID NO:4. In some embodiments, the 3′ inverted terminal repeat sequence has 100% identity to the entire length of SEQ ID NO:4. In some embodiments, the 3′ inverted terminal repeat sequence consists of to the entire length of SEQ ID NO:4.

Inverted terminal repeats, such as transposon-specific inverted terminal repeat sequences can be recognized by a transposase, such as the Super piggyBac transposase. The piggyBac (PB) transposon is a mobile genetic element that efficiently transposes between vectors and chromosomes via a “cut and paste” mechanism. During transposition, the Super PB transposase recognizes transposon-specific inverted terminal repeat sequences (ITRs) located on both ends of the transposon vector and moves the contents from the original sites and efficiently integrates them into TTAA chromosomal sites. See, e.g., System Biosciences, 2014, “PiggyBac Transposon System.”

In some embodiments, disclosed methods comprise integrating an expression cassette into the genome of a host organism, where the expression cassette further comprises a promoter. In some embodiments, the promoter flanks the nucleotide sequence encoding the interfering RNA molecule on the 5′ end. In some embodiments, the promoter is operably linked to the nucleotide sequence encoding the interfering RNA molecule on the 5′ end. In some embodiments, the promoter separates the nucleotide sequence encoding the interfering RNA molecule from the 5′ inverted terminal repeat sequence. In some embodiments, the promoter is a glyceraldehyde-3-phosphate dehydrogenase (GAP) promoter or a derivative thereof.

In some embodiments, the GAP promoter comprises a nucleotide sequence with at least 70%, 75%, 80%, 85%, 90%, 95%, 99%, or 100% identity to the entire length of SEQ ID NO:11. In some embodiments, the GAP promoter comprises a nucleotide sequence with 70%, 75%, 80%, 85%, 90%, 95%, 99%, or 100% identity to the entire length of SEQ ID NO:11. In some embodiments, the GAP promoter comprises a nucleotide sequence with 100% identity to the entire length of SEQ ID NO:11. In some embodiments, the GAP promoter consists of a nucleotide sequence with 100% identity to the entire length of SEQ ID NO:11.

In some embodiments, disclosed methods comprise integrating an expression cassette into the genome of a host organism, where the expression cassette further comprises a terminator, such as a transcription terminator. In some embodiments, the terminator flanks the nucleotide sequence encoding the interfering RNA molecule on the 3′ end. In some embodiments, the terminator separates the nucleotide sequence encoding the interfering RNA molecule from the 3′ inverted terminal repeat sequence. In some embodiments, the terminator is a CYC1 terminator.

In some embodiments, the CYC1 terminator comprises a nucleotide sequence with at least 70%, 75%, 80%, 85%, 90%, 95%, 99%, or 100% identity to the entire length of SEQ ID NO:12. In some embodiments, the CYC1 terminator comprises a nucleotide sequence with 70%, 75%, 80%, 85%, 90%, 95%, 99%, or 100% identity to the entire length of SEQ ID NO:12. In some embodiments, the CYC1 terminator comprises a nucleotide sequence with 100% identity to the entire length of SEQ ID NO:12. In some embodiments, the CYC1 terminator consists of a nucleotide sequence with 100% identity to the entire length of SEQ ID NO:12.

In some embodiments, disclosed methods comprise integrating an expression cassette into the genome of a host organism, where the expression cassette further comprises a selection marker promoter operably linked to a selection marker gene. In some embodiments, the selection marker is an auxotrophic marker, such as an auxotrophic rescue gene. In some embodiments, the selection marker promoter is flanked by the 5′ inverted terminal repeat. In some embodiments, the selection marker promoter is a minimal promoter. In some embodiments, the minimal promoter is truncated or mutated. In some embodiments, the selection marker promoter is a minimal auxotrophic rescue promoter. Herein, an “auxotrophic rescue promoter” may be interchangeably referred to as an “auxotrophic marker promoter.”

In some embodiments, disclosed methods comprise integrating an expression cassette into the genome of a host organism, where the expression cassette further comprises an auxotrophic rescue promoter operably linked to an auxotrophic rescue gene. In some embodiments, the auxotrophic rescue promoter is flanked by the 5′ inverted terminal repeat. In some embodiments, the auxotrophic rescue promoter is a minimal auxotrophic rescue promoter. In some embodiments, the minimal auxotrophic rescue promoter is truncated or mutated.

In some embodiments, the minimal auxotrophic rescue promoter is a truncated leucine promoter and the auxotrophic rescue gene is leucine. In some embodiments, the minimal promoter comprises a nucleotide sequence with at least 70%, at least 80%, or at least 90% identity to the entire length of SEQ ID NO:3. In some embodiments, the minimal promoter comprises a nucleotide sequence with 70%, 73%, 77%, 80%, 83%, 87%, 90%, 93%, 97% or 100% identity to the entire length of SEQ ID NO:3. In some embodiments, the minimal promoter comprises a nucleotide sequence with 100% identity to the entire length of SEQ ID NO:3. In some embodiments, the minimal promoter consists of a nucleotide sequence with 100% identity to the entire length of SEQ ID NO:3.

In some embodiments, the insulator sequence separates the 5′ end of the nucleotide sequence encoding the interfering RNA molecule from the 5′ inverted terminal repeat sequence. In some embodiments, the insulator sequence separates the 3′ end of the nucleotide sequence encoding the interfering RNA molecule from the 3′ inverted terminal repeat sequence.

In some embodiments, disclosed methods comprise integrating an expression cassette into the genome of a host organism, where the expression cassette further comprises an insulator sequence with at least 70%, 75%, 80%, 85%, 90%, 95%, 99%, or 100% identity to the entire length of SEQ ID NO:6. In some embodiments, the insulator sequence has about 70%, 75%, 80%, 85%, 90%, 95%, 98%, 99%, or 100% identity to the entire length of SEQ ID NO:6. In some embodiments, the insulator sequence consists of a nucleotide sequence with 100% identity to the entire length of SEQ ID NO:6.

In some embodiments, disclosed methods comprise integrating an expression cassette into the genome of a host organism, where the expression cassette comprises at least one, three, five, seven, nine, or ten copies of the nucleotide sequence encoding the interfering RNA molecule. In some embodiments, the expression cassette comprises one, two, three, four, five, six, seven, eight, nine, or ten copies of the nucleotide sequence encoding the interfering RNA molecule.

In some embodiments, disclosed methods comprise integrating an expression cassette into the genome of a host organism, wherein the expression cassette comprises a nucleotide sequence encoding an interfering RNA molecule that inhibits the expression of a gene in Aedes spp., Anopheles spp., or Culex spp. In some embodiments, the interfering RNA molecule inhibits the expression of a gene in Anopheles gambiae, Aedes aegypti, Aedes albopictus, Culex pipiens, or Culex quinquesfasciatus. In some embodiments, the interfering RNA molecule inhibits the expression of Shaker in an Aedes spp., Anopheles spp., or Culex spp., e.g., Anopheles gambiae, Aedes aegypti, Aedes albopictus, Culex pipiens, or Culex quinquesfasciatus.

In some embodiments, disclosed methods comprise integrating an expression cassette into the genome of a host organism, wherein the expression cassette comprises a nucleotide sequence which encodes an interfering RNA molecule that targets the gene Shaker in the mosquito. In some embodiments, the interfering RNA molecule is capable of inhibiting the expression of Shaker in the mosquito. In some embodiments, a nucleotide sequence transcribed from the nucleotide sequence encoding the interfering RNA molecule is partially or perfectly complementary to a sequence comprising at least 80%, 84%, 88%, 92%, 96%, or 100% identity to the entire length of SEQ ID NO: 2. In some embodiments, the interfering RNA molecule comprises at least 25 contiguous nucleotides which are partially or perfectly complementary to the entire length of SEQ ID NO: 2. In some embodiments, the nucleotide sequence encoding the interfering RNA molecule is integrated, such as stably integrated, into the genome of the host organism by transposition.

In some embodiments, disclosed methods comprise integrating an expression cassette into the genome of a host organism, wherein the expression cassette comprises a nucleotide sequence encoding the interfering RNA molecule shRNA.463, also referred to herein as “shRNA_463,” which is represented by SEQ ID NO:1, wherein the nucleotide sequence is flanked by an inverted terminal repeat sequence. In some embodiments, the expression cassette comprises a nucleotide sequence that has at least 80%, 85%, 90%, 95%, or 100%, identity to SEQ ID NO:1, wherein the nucleotide sequence encodes an interfering RNA molecule and is flanked by an inverted terminal repeat sequence. In some embodiments, the nucleotide sequence has 80%, 82%, 84%, 86%, 88%, 90%, 92%, 94%, 96%, 98%, or 100% identity to the entire length of SEQ ID NO:1, wherein the nucleotide sequence encodes an interfering RNA molecule and is flanked by an inverted terminal repeat sequence. In some embodiments, the nucleotide sequence encoding the interfering RNA molecule consists of 100% identity to the entire length of SEQ ID NO:1, wherein the nucleotide sequence encodes an interfering RNA molecule and is flanked by an inverted terminal repeat sequence. In some embodiments, the inverted terminal repeat sequence flanks the 5′ end and the 3′ end of the nucleotide sequence encoding the interfering RNA molecule.

In some embodiments, disclosed methods comprise integrating an expression cassette into the genome of a host organism, wherein the expression cassette comprises a nucleotide sequence with 80%, 82%, 84%, 86%, 88%, 90%, 92%, 94%, 96%, 98%, or 100% identity to the entire length of SEQ ID NO:1 flanked on the 5′ end by an inverted terminal repeat sequence with about 70%, 75%, 80%, 85%, 90%, 95%, 99%, or 100% identity to the entire length of SEQ ID NO:4. In some embodiments, the nucleotide sequence has 80%, 82%, 84%, 86%, 88%, 90%, 92%, 94%, 96%, 98%, or 100% identity to the entire length of SEQ ID NO:1 flanked on the 3′ end by an inverted terminal repeat sequence with about 70%, 75%, 80%, 85%, 90%, 95%, 99%, or 100% identity to the entire length of SEQ ID NO:5.

In some embodiments, disclosed methods comprise integrating an expression cassette into the genome of a host organism, wherein the expression cassette comprises at least one, three, five, seven, nine, or ten copies of the nucleotide sequence encoding the interfering RNA molecule, e.g., SEQ ID NO:1, or a sequence having at least 80% or 90% identity to SEQ ID NO:1.

In some embodiments, disclosed methods comprise integrating an expression cassette into the genome of a host organism, wherein the expression cassette comprises a nucleotide sequence with at least about 70%, 75%, 80%, 85%, 90%, 95%, 98%, 99%, or 100% identity to the entire length of SEQ ID NO:7 or the complement thereof. In some embodiments, the expression cassette comprises a nucleotide sequence with at least about 70%, 75%, 80%, 85%, 90%, 95%, 98%, 99%, or 100% identity to the entire length of SEQ ID NO:8 or the complement thereof.

In some embodiments, the expression cassette comprises a nucleotide sequence with about 70%, 75%, 80%, 85%, 90%, 95%, 98%, 99%, or 100% identity to the entire length of SEQ ID NO:7 or the complement thereof. In some embodiments, the expression cassette comprises a nucleotide sequence with about 70%, 75%, 80%, 85%, 90%, 95%, 98%, 99%, or 100% identity to the entire length of SEQ ID NO:8 or the complement thereof.

In some embodiments, disclosed methods comprise integrating an expression cassette comprising a nucleotide sequence encoding an interfering RNA molecule into the genome of a host organism with a transposase comprising a nucleotide sequence with at least 70%, 75%, 80%, 85%, 90%, 95%, or 100% identity to the entire length of SEQ ID NO:10. In some embodiments, the transposase comprises a nucleotide sequence with about 70%, 75%, 80%, 85%, 90%, 95%, 97%, 98%, 99%, or 100% identity to the entire length of SEQ ID NO:10.

The Super piggyBac transposase is represented by SEQ ID NO:10. The activity of the piggyBac transposon system enables genes of interest between the two (5′ and 3′) inverted terminal repeat sequences in the piggyBac vector to be easily mobilized into target genomes. In some embodiments, the Super piggyBac transposase is operably linked to a constitutive promoter. In some embodiments, the Super piggyBac transposase is operably linked to an ADH1 promoter, such as a strong ADH1 promoter or a derivative thereof, such as a truncated version. SEQ ID NO:21 represents the full length ADH1 promoter. In some embodiments, SEQ ID NO:10 or a nucleotide sequence having at least 70%, at least 80%, at least 90%, or at least 95% to the entire length SEQ ID NO:10 of is operably linked to an ADH1 promoter or a derivative thereof, such as a truncated ADH1 promoter.

In some embodiments, disclosed methods comprise integrating an expression cassette comprising a nucleotide sequence encoding an interfering RNA molecule into the genome of a microbial host organism, such as a fungal cell. In some embodiments, the microbial host cell is a yeast cell. In some embodiments, the host organism is a species of Pichia, e.g., Pichia pastoris, a species of Saccharomyces, e.g., Saccharomyces cerevisiae, or a species of Yarrowia, e.g., Yarrowia lipolytica. In some embodiments, the host organism is Saccharomyces cerevisiae. In some embodiments, the host organism is Saccharomyces cerevisiae strain FL100 or Saccharomyces cerevisiae strain S288C.

In some embodiments, disclosed methods comprise integrating an expression cassette comprising a nucleotide sequence encoding an interfering RNA molecule into the genome of S. cerevisiae, such as strain FL100, at any one or more of the integration sites chromosome IV (NC_001136 at position 543,705); chromosome IV (NC_001136 position 1,357,520); chromosome VIII (NC_001140 position 124,029); chromosome X (NC_001142 position 181,309); chromosome XI (NC_001143 position 300,654); and chromosome XII (NC_001144 position 213,991).

Confirming the integration of expression cassettes into the genome of a host organism may be accomplished according to methods known to one of skill in the art. For example, PCR and sequencing may be used to confirm integration of expression cassettes, such as expression cassettes comprising a nucleotide sequence encoding an interfering RNA molecule.

Specifically relating to the shRNA Sh.4643, expression may be confirmed after total RNA extraction using TRIzol Reagent according to the manufacturer's instructions (Invitrogen, Carlsbad, CA) from 5.6 mg of pelleted yeast taken from cultures that had been prepared as described by Hapairai et al., Sci Rep. 2017; 7(1):13223. cDNA can be prepared according to the instructions provided in the High Capacity RNA to cDNA Kit (Applied Biosystems, Foster City, CA), and 1/100 of the resulting cDNA may be used as template for PCR amplifications performed with Clontech Labs 3P TaKaRa Taq DNA Polymerase (Clontech Laboratories, Mountain View, CA), as described by Mysore et al., PLoS Negl Trop Dis. 2020 July; 14(7): e0008479.

### Genetically Modified Host Organisms and Elements Thereof

In some aspects, provided herein are polynucleotide sequences and genetically modified host organisms comprising the same which are designed to produce interfering RNA molecules, such as interfering RNA pesticides. Such interfering RNA pesticides are capable of interfering with the expression of a gene in a mosquito, such as inhibiting gene expression of the target gene in the mosquito.

### Polynucleotide Sequences and Expression Cassettes

In some aspects, provided herein are nucleotide sequences and expression cassettes comprising a nucleotide sequence encoding an interfering RNA molecule which is capable of inhibiting the expression of a gene in a mosquito. In some embodiments, the nucleotide sequence encoding the interfering RNA molecule is flanked by an inverted terminal repeat. In some embodiments, the nucleotide sequence encoding the interfering RNA molecule is flanked by a 5′ inverted terminal repeat sequence and a ′3 inverted terminal repeat sequence. In some embodiments, the interfering RNA molecule is an RNA construct, a double stranded RNA (dsRNA), a small interfering RNA (siRNA), a short hairpin RNA (shRNA), or an anti-sense oligonucleotide.

In some embodiments, the 5′ inverted terminal repeat sequence has at least about 70%, 75%, 80%, 85%, 90%, 95%, 99%, or 100% identity to the entire length of SEQ ID NO:5. In some embodiments, the 5′ inverted terminal repeat sequence has about 70%, 75%, 80%, 85%, 90%, 95%, 99%, or 100% identity to the entire length of SEQ ID NO:5. In some embodiments, the 5′ inverted terminal repeat sequence has 100% identity to the entire length of SEQ ID NO:5. In some embodiments, the 5′ inverted terminal repeat sequence consists of to the entire length of SEQ ID NO:5.

In some embodiments, the 3′ inverted terminal repeat sequence has at least about 70%, 75%, 80%, 85%, 90%, 95%, 99%, or 100% identity to the entire length of SEQ ID NO:4. In some embodiments, the 3′ inverted terminal repeat sequence has about 70%, 75%, 80%, 85%, 90%, 95%, 99%, or 100% identity to the entire length of SEQ ID NO:4. In some embodiments, the 3′ inverted terminal repeat sequence has 100% identity to the entire length of SEQ ID NO:4. In some embodiments, the 3′ inverted terminal repeat sequence consists of to the entire length of SEQ ID NO:4.

In some embodiments, disclosed expression cassettes comprise inverted terminal repeats. The piggyBac inverted terminal repeat element is a short inverted terminal repeat (ITR) transposable element, 2.5 kb long, with 13-bp ITR sequences and a 2.1-kb ORF. See, e.g., Handler et al., PNAS 1998; 95(13): 7520-7525; Cary et al. Virology 1989; 161, 8-17; Elick et al. Genetica 1995; 97, 127-139). It is part of a subclass of ITR elements that are thus far found only in lepidopterans and that insert exclusively into TTAA target sites. See, e.g., Beames & Summers, Virology 1990; 174: 354-363; Fraser et al., Virology 1995; 211: 397-407; Wang & Fraser, Insect Mol Biol 1993; 1: 109-116.

In some embodiments, disclosed expression cassettes further comprise a promoter. In some embodiments, the promoter flanks the nucleotide sequence encoding the interfering RNA molecule on the 5′ end. In some embodiments, the promoter is operably linked to the nucleotide sequence encoding the interfering RNA molecule on the 5′ end. In some embodiments, the promoter separates the nucleotide sequence encoding the interfering RNA molecule from the 5′ inverted terminal repeat sequence. In some embodiments, the promoter is a glyceraldehyde-3-phosphate dehydrogenase (GAP) promoter.

A “promoter” is a control sequence that is a region of a nucleic acid sequence at which initiation and rate of transcription are controlled. It may contain genetic elements at which regulatory proteins and molecules may bind, such as RNA polymerase and transcription factors, to initiate the transcription of a nucleic acid sequence. The phrase “operably linked” indicates that an expression control element, e.g., a promoter, is in an appropriate location and/or orientation in relation to a nucleic acid to control transcriptional initiation and/or expression of the nucleic acid.

A promoter may be one that is naturally associated with a nucleic acid sequence, as may be obtained by isolating the 5′ non-coding sequences located upstream of the coding segment. Alternatively, a promoter may be a recombinant or heterologous promoter, which refers to a promoter that is not normally associated with a nucleic acid segment in its natural environment. Such promoters may include promoters of other genes and promoters that are not naturally occurring. An expression control element may be derived from a yeast of the species or strain in which RNA interference (RNAi) is to be used or in which the RNAi pathway is to be engineered. For example, if RNAi is to be used in S. cerevisiae, it may be desirable to use a S. cerevisiae promoter to direct expression of a dsRNA. However, any expression control element capable of directing transcription in the cell of interest may be used.

The promoters employed may be either constitutive or inducible. For example, various yeast-specific promoters may be employed to regulate the expression in yeast cells. Examples of inducible yeast promoters include GAL1-10, GAL1, GALL, GALS, TET, CUP1, VP16 and VP16-ER. Examples of repressible yeast promoters include Met25. Examples of constitutive yeast promoters include glyceraldehyde 3-phosphate dehydrogenase promoter (also known as GAP, GPD, and TDH3), phosphoglycerate kinase (PGK), alcohol dehydrogenase promoter (ADH), translation-elongation factor-1-alpha promoter (TEF), cytochrome c-oxidase promoter (CYC1), and MRP7. Promoters containing steroid response elements (e.g., glucocorticoid response element) inducible by glucocorticoid or other steroid hormones can also direct expression in yeast. Yet other yeast constitutive or inducible promoters such as those of the genes for alpha factor, phosphate pathway genes (e.g., PH05), or alcohol oxidase may be used. In some embodiments, the vector comprises an expression control element known as an upstream activating sequence (UAS).

Such elements, which are considered functional equivalents of metazoan enhancers, can activate gene transcription from remote positions, e.g., up to about 1,000 to 1,200 bp from the promoter. See, e.g., Petrascheck, M, et al., Nucleic Acids Res., 33(12): 3743-3750, 2005, for discussion. The level of expression achieved using an inducible promoter can be regulated, e.g., by controlling the amount of inducing agent or the length of exposure. Further, mutant promoters that result in lower expression levels than a wild-type promoter can be used. In some embodiments, an expression control element originates from a species in which the expression control element is to be used to direct expression while in other embodiments the expression control element originates from a different species.

Suitable promoters for expression in yeast are also well known and include, for example, the bacteriophage T7 promoter, promoters from GAL1 (which is induced by the presence of galactose), ADH1, the TEF1 promoter and the AOX promoter (a methanol inducible promoter), and the like. Many yeast cloning vectors have been designed and are readily available. Methods for transforming S. cerevisiae cells with exogenous DNA and producing recombinant products polypeptides are also well known. Transformed cells are selected by phenotype determined by a selectable marker, commonly drug resistance or the ability to grow in the absence of a particular nutrient (e.g., leucine). Applications and use of the GAP promoter have been described in, e.g., Waterham et al., Gene. 1997 Feb. 20; 186(1):37-44.

Certain promoters may also be utilized in the expression of small RNAs, such as single guide RNA (sgRNA) involved in specifying a target sequence for endonuclease activity. One of methods of making sgRNAs involves expressing guide RNA cloned into a plasmid vector in host cells. In some examples, the cells use their normal RNA polymerase enzyme to transcribe the genetic information in the newly introduced DNA to generate the sgRNA. In some embodiments, a nucleotide sequence encoding an sgRNA is operably linked to a promoter, such as an RNA polymerase promoter, a tRNA promoter, or a hybrid RNA polymerase-tRNA promoter. In some embodiments, the RNA polymerase promoter is an RNA pol II or an RNA pol III promoter. In some embodiments, the RNA pol II promoter is an ADH promoter. In some embodiments, the RNA pol III promoter is an SNR52 promoter or a derivative thereof.

In some embodiments, disclosed expression cassettes comprise nucleotide sequences encoding gRNA, such as sgRNA, and promoters and terminators for the same. In some embodiments, the SNR52 promoter has at least about 70%, 75%, 80%, 85%, 90%, 95%, 99%, or 100% identity to the entire length of to the entire length of SEQ ID NO:13. In some embodiments, the SNR52 promoter has about 70%, 75%, 80%, 85%, 90%, 95%, 99%, or 100% identity to the entire length of to the entire length of SEQ ID NO:13. In some embodiments, the SNR52 promoter consists of the entire length of SEQ ID NO:13.

The SNR52 promoter is unique amongst Pol III promoters in that it has native cleavage sites that result in the excision of sgRNAs from the primary transcripts. See, e.g., DiCarlo et al., Nucleic Acids Res. 2013; 41: 4336-4343 and Marck et al., Nucleic Acids Res. 2006; 34 (6):1816-1835. In some examples, a promoter operably linked to a nucleotide sequence encoding sgRNA may be optimized improve transcription efficiency and mutation frequency, e.g., as described by Ng & Dean, mSphere. 2017 March-April; 2(2): e00385-16 and Schwartz et al., ACS Synth. Biol. 2016, 5, 4, 356-359.

In some embodiments, the nucleotide sequence terminating transcription of the sequence encoding gRNA, such as a gRNA terminator. In some embodiments, the gRNA terminator is an SNR52 terminator. In some embodiments, the SNR52 terminator comprises a nucleotide sequence with at least 80%, 85%, 90%, 95%, 99%, or 100% identity to the entire length of SEQ ID NO:14. In some embodiments, the SNR52 terminator comprises a nucleotide sequence with about 80%, 85%, 90%, 95%, 99%, or 100% identity to the entire length of SEQ ID NO:14. In some embodiments, the SNR52 terminator comprises a nucleotide sequence with 100% identity to the entire length of SEQ ID NO:14. In some embodiments, the SNR52 terminator consists of a nucleotide sequence with 100% identity to the entire length of SEQ ID NO:14.

In some embodiments, disclosed expression cassettes further comprise a promoter sequence, such as a GAP promoter. In some embodiments, the GAP promoter comprises a nucleotide sequence with at least 70%, 75%, 80%, 85%, 90%, 95%, 99%, or 100% identity to the entire length of SEQ ID NO:11. In some embodiments, the GAP promoter comprises a nucleotide sequence with 70%, 75%, 80%, 85%, 90%, 95%, 99%, or 100% identity to the entire length of SEQ ID NO:11. In some embodiments, the GAP promoter comprises a nucleotide sequence with 100% identity to the entire length of SEQ ID NO:11. In some embodiments, the GAP promoter consists of a nucleotide sequence with 100% identity to the entire length of SEQ ID NO:11.

In some embodiments, disclosed expression cassettes further comprise a terminator, such as a transcription terminator. In some embodiments, the terminator flanks the nucleotide sequence encoding the interfering RNA molecule on the 3′ end. In some embodiments, the terminator separates the nucleotide sequence encoding the interfering RNA molecule from the 3′ inverted terminal repeat sequence. In some embodiments, the terminator is a CYC1 terminator or a derivative thereof.

In some embodiments, disclosed expression cassettes comprise a CYC1 terminator comprising a nucleotide sequence with at least 70%, 75%, 80%, 85%, 90%, 95%, 99%, or 100% identity to the entire length of SEQ ID NO:12. In some embodiments, disclosed expression cassettes comprise a CYC1 terminator comprising a nucleotide sequence with 70%, 75%, 80%, 85%, 90%, 95%, 99%, or 100% identity to the entire length of SEQ ID NO:12. In some embodiments, disclosed expression cassettes comprise a CYC1 terminator comprising a nucleotide sequence with 100% identity to the entire length of SEQ ID NO:12. In some embodiments, disclosed expression cassettes comprise a CYC1 terminator consisting of a nucleotide sequence with 100% identity to the entire length of SEQ ID NO:12.

The CYC1 terminator is a DNA fragment with a special structure, marking the end of transcription, which is located in Saccharomyces cerevisiae strain FL100 chromosome X from 526690 bp to 526939 bp. Synthetic terminators, including CYC1 terminators and derivatives thereof, for heterologous gene expression in yeast are described in, e.g., Curran et al., ACS Synthetic Biology, 2015; 4(7):824-832, Mumberg et al., Gene. 1995 Apr. 14; 156(1):119-22, Zaret & Sherman, Cell. 1982 March; 28(3):563-73.

In some embodiments, disclosed expression cassettes comprise nucleotide sequences encoding gRNA, such as sgRNA. In some embodiments, the nucleotide sequence terminating transcription of the sequence encoding gRNA, such as a gRNA terminator, is an SNR52 terminator or a derivative thereof. In some embodiments, disclosed expression cassettes comprise an SNR52 terminator comprising a nucleotide sequence with at least 80%, 85%, 90%, 95%, 99%, or 100% identity to the entire length of SEQ ID NO:14. In some embodiments, disclosed expression cassettes comprise an SNR52 terminator comprising a nucleotide sequence with about 80%, 85%, 90%, 95%, 99%, or 100% identity to the entire length of SEQ ID NO:14. In some embodiments, disclosed expression cassettes comprise an SNR52 terminator comprising a nucleotide sequence with 100% identity to the entire length of SEQ ID NO:14. In some embodiments, disclosed expression cassettes comprise an SNR52 terminator consisting of a nucleotide sequence with 100% identity to the entire length of SEQ ID NO:14.

In some embodiments, disclosed expression cassettes further comprise a selection marker promoter operably linked to a selection marker gene. In some embodiments, the selection marker is an auxotrophic marker, such as an auxotrophic rescue gene. In some embodiments, the selection marker promoter is flanked by the 5′ inverted terminal repeat. In some embodiments, the selection marker promoter is a minimal promoter. In some embodiments, the minimal promoter is truncated or mutated. In some embodiments, the selection marker promoter is a minimal auxotrophic rescue promoter.

In some embodiments, disclosed expression cassettes further comprise an auxotrophic rescue promoter operably linked to an auxotrophic rescue gene. In some embodiments, the auxotrophic rescue promoter is flanked by the 5′ inverted terminal repeat. In some embodiments, the auxotrophic rescue promoter is a minimal auxotrophic rescue promoter. In some embodiments, the minimal auxotrophic rescue promoter is truncated or mutated.

In some embodiments, the minimal auxotrophic rescue promoter is a truncated leucine promoter and the auxotrophic rescue gene is leucine. In some embodiments, the minimal promoter comprises a nucleotide sequence with at least 70%, at least 80%, or at least 90% identity to the entire length of SEQ ID NO:3. In some embodiments, the minimal promoter comprises a nucleotide sequence with 70%, 73%, 77%, 80%, 83%, 87%, 90%, 93%, 97% or 100% identity to the entire length of SEQ ID NO:3. In some embodiments, the minimal promoter comprises a nucleotide sequence with 100% identity to the entire length of SEQ ID NO:3. In some embodiments, the minimal promoter consists of a nucleotide sequence with 100% identity to the entire length of SEQ ID NO:3.

In some embodiments, disclosed expression cassettes comprising a minimal or truncated promoter operably linked to a selection marker gene increase expression levels of interfering RNA molecules relative to an expression cassette comprising a native or full-length promoter operably linked to the selection marker gene. In some embodiments, expression levels of the interfering RNA molecules are increased by 25%, 50%, 75%, 100%, 125%, 150%, 175%, 200%, 225%, 250%, 275%, 300%, 325%, 350%, 375%, 400%, 425%, 450%, 475%, or 500%.

In some embodiments, expression levels of the interfering RNA molecules are increased by at least 25%, 50%, 75%, 100%, 125%, 150%, 175%, 200%, 225%, 250%, 275%, 300%, 325%, 350%, 375%, 400%, 425%, 450%, 475%, or 500% relative to an expression cassette comprising a native promoter operably linked to the selection marker gene. In some embodiments, expression levels of the interfering RNA molecules are increased by about 25%, 50%, 75%, 100%, 125%, 150%, 175%, 200%, 225%, 250%, 275%, 300%, 325%, 350%, 375%, 400%, 425%, 450%, 475%, or 500% relative to an expression cassette comprising a native promoter operably linked to the selection marker gene. In some embodiments, expression levels are 5 times greater, 10 times greater, 15 times greater, 20 times greater, 25 times greater, 30 times greater, 35 times greater, 40 times greater, 45 times greater, or 50 times greater relative to an expression cassette comprising a native promoter operably linked to the selection marker gene.

Use of an integrated expression cassette having a minimal, defective, or truncated promoter as described herein resulted in increased expression of the interfering RNA molecule relative to use of a native promoter driving expression of the auxotrophic rescue gene. In comparison to native promoters, minimal promoters may be truncated or mutated. See, e.g, Parker & Newstead, Protein Sci. 2014 September; 23(9):1309-14 and Tang et al., Metabolites. 2020 Aug. 6; 10(8):320. The truncation of endogenous promoters to remove non-essential bases is one strategy for minimal promoter construction. However, minimal truncated promoters contain elements from endogenous promoters and may be subject to homologous recombination. Accordingly, mutagenesis, such as saturation mutagenesis, is another strategy for creating minimal promoters See, e.g., Tang et al., Metabolites. 2020 Aug. 6; 10(8):320.

In some embodiments, disclosed expression cassettes further comprise a selection rescue gene. In some embodiments, the selection marker gene is an auxotrophic rescue gene, such as an auxotrophic marker gene that complements an auxotrophic mutation. In some embodiments, the auxotrophic rescue gene encodes an amino acid, such as an essential amino acid. In some embodiments, the essential amino acid is histidine, leucine, tryptophan, uracil, or a combination thereof. In some embodiments, the auxotrophic rescue gene is leu2, ura3, his3, trp1, orthologs thereof, or a combination thereof. In some embodiments, disclosed expression cassettes comprise the auxotrophic rescue promoter dleu2, which is represented by SEQ ID NO:3, operably linked to leu2.

Use of auxotrophic mutant host organisms facilitates selection of cells which have had a disclosed expression cassette successfully integrated into the host cell genome, i.e., cell growth is indicative of auxotroph complementation or restored prototrophy, such as by transposon-mediated genome integration of disclosed expression cassettes. Accordingly, the selection of an auxotrophic rescue promoter and an auxotrophic rescue gene relate to the auxotrophic mutations. Auxotrophic genes are required for growth in the absence of an essential nutrient, such as an essential amino acid. For example, the auxotrophic URA3 gene mutation results in failure to grow in a medium that lacks uracil, given that URA3 encodes a key enzyme in the de novo pathway for uracil biosynthesis (Lacroute, J Bacteriol 95:824-832). Growth of ura3 mutants in uracil-deficient medium can be restored, if the cells have been transformed with DNA containing the URA3 gene, such as by transposition, as described herein. Additional selection and counterselection techniques are described in, e.g., Yuan, PLoS ONE 2011; 6(10):e25830.

In some embodiments, disclosed expression cassettes further comprise an insulator sequence. In some embodiments, the insulator sequence separates the 5′ end of the nucleotide sequence encoding the interfering RNA molecule from the 5′ inverted terminal repeat sequence. In some embodiments, the insulator sequence separates the 3′ end of the nucleotide sequence encoding the interfering RNA molecule from the 3′ inverted terminal repeat sequence.

In some embodiments, disclosed expression cassettes comprise an insulator sequence with at least 70%, 75%, 80%, 85%, 90%, 95%, 99%, or 100% identity to the entire length of SEQ ID NO:6. In some embodiments, disclosed expression cassettes comprise an insulator sequence with about 70%, 75%, 80%, 85%, 90%, 95%, 98%, 99%, or 100% identity to the entire length of SEQ ID NO:6. In some embodiments, the insulator sequence consists of a nucleotide sequence with 100% identity to the entire length of SEQ ID NO:6.

Insulators are genome sequence elements that help to organize eukaryotic genomes into coherent regulatory domains. Insulators can encode both enhancer-blocking activity, which prevents the interaction between enhancers and promoters located in distinct regulatory domains, and/or chromatin barrier activity that helps to delineate active and repressive chromatin domains. Accordingly, insulators are cis-regulatory elements that can block improper gene activation or heterochromatin propagation imposed by remote enhancers and silencers. Insulators thus have been applied to the stabilization of transgenes, such as those delivered by viral vectors. See, e.g., Wang et al., Proc Natl Acad Sci USA. 2015 Aug. 11; 112(32):E4428-37. Insulator sequences have also been described in the context of transposition, or transposon-mediated gene delivery. See, e.g., Bire et al., PLoS One. 2013; 8(12):1-10 and Mossine et al., PLoS One. 2013; 8(12): e85494.

In some embodiments, disclosed expression cassettes encode an interfering RNA molecule that inhibits the expression of a gene in a mosquito that is a species of Aedes, Anopheles, or Culex. In some embodiments, disclosed expression cassettes comprises a nucleotide sequence encoding an interfering RNA molecule that inhibits the expression of a gene in Anopheles gambiae, Aedes aegypti, Aedes albopictus, Culex pipiens, or Culex quinquesfasciatus. In some embodiments, disclosed expression cassette comprise a nucleotide sequence encoding an interfering RNA molecule that inhibits the expression of Shaker in an Aedes spp., Anopheles spp., or Culex spp., e.g., Anopheles gambiae, Aedes aegypti, Aedes albopictus, Culex pipiens, or Culex quinquesfasciatus.

In some embodiments, disclosed expression cassettes comprise a nucleotide sequence encoding an interfering RNA molecule, wherein the interfering RNA molecule is an RNA construct, a double stranded RNA (dsRNA), a small interfering RNA (siRNA), a short hairpin RNA (shRNA), or an anti-sense oligonucleotide. In some embodiments, disclosed expression cassettes comprise inverted terminal repeat sequences flanking the nucleotide sequence encoding the interfering RNA molecule.

In some embodiments, disclosed expression cassettes comprise a nucleotide sequence which encodes an interfering RNA molecule that targets the gene Shaker in the mosquito. In some embodiments, the interfering RNA molecule is capable of inhibiting the expression of Shaker in the mosquito. In some embodiments, a nucleotide sequence transcribed from the nucleotide sequence encoding the interfering RNA molecule is partially or perfectly complementary to a sequence comprising at least 80%, 84%, 88%, 92%, 96%, or 100% identity to the entire length of SEQ ID NO: 2. In some embodiments, disclosed expression cassettes encode an interfering RNA molecule which comprises at least 25 contiguous nucleotides which are partially or perfectly complementary to the entire length of SEQ ID NO: 2. In some embodiments, the nucleotide sequence encoding the interfering RNA molecule is integrated, such as stably integrated, into the genome of the host organism by transposition.

In some embodiments, disclosed expression cassettes comprise a nucleotide sequence encoding the interfering RNA molecule shRNA.463, which is represented by SEQ ID NO:1, wherein the nucleotide sequence is flanked by an inverted terminal repeat sequence. In some embodiments, disclosed expression cassettes comprise a nucleotide sequence encoding an interfering RNA molecule that consists of 100% identity to the entire length of SEQ ID NO:1, wherein the nucleotide sequence encodes an interfering RNA molecule and is flanked by an inverted terminal repeat sequence. In some embodiments, the inverted terminal repeat sequence flanks the 5′ end and the 3′ end of the nucleotide sequence encoding the interfering RNA molecule.

In some embodiments, disclosed expression cassettes comprise a nucleotide sequence that has at least 80%, 85%, 90%, 95%, or 100%, identity to SEQ ID NO:1, wherein the nucleotide sequence encodes an interfering RNA molecule and is flanked by an inverted terminal repeat sequence. In some embodiments, disclosed expression cassettes comprise a nucleotide sequence that has 80%, 82%, 84%, 86%, 88%, 90%, 92%, 94%, 96%, 98%, or 100% identity to the entire length of SEQ ID NO:1, wherein the nucleotide sequence encodes an interfering RNA molecule and is flanked by an inverted terminal repeat sequence. The main portion of SEQ ID NO:1 that could likely be modified without compromising the insecticidal activity is TTCAAGAGA, which corresponds to the hairpin loop.

In some embodiments, disclosed expression cassettes comprise a nucleotide sequence with 80%, 82%, 84%, 86%, 88%, 90%, 92%, 94%, 96%, 98%, or 100% identity to the entire length of SEQ ID NO:1 flanked on the 5′ end by an inverted terminal repeat sequence with about 70%, 75%, 80%, 85%, 90%, 95%, 99%, or 100% identity to the entire length of SEQ ID NO:4. In some embodiments, disclosed expression cassettes comprise a nucleotide sequence with 80%, 82%, 84%, 86%, 88%, 90%, 92%, 94%, 96%, 98%, or 100% identity to the entire length of SEQ ID NO:1 flanked on the 3′ end by an inverted terminal repeat sequence with about 70%, 75%, 80%, 85%, 90%, 95%, 99%, or 100% identity to the entire length of SEQ ID NO:5.

In some embodiments, disclosed expression cassettes comprise at least one, three, five, seven, nine, or ten copies of the nucleotide sequence encoding the interfering RNA molecule, e.g., SEQ ID NO:1. In some embodiments, disclosed expression cassettes comprise at least one, three, five, seven, nine, or ten copies of the nucleotide sequence encoding the interfering RNA molecule. In some embodiments, disclosed expression cassettes comprise one, two, three, four, five, six, seven, eight, nine, or ten copies of the nucleotide sequence encoding the interfering RNA molecule.

In some embodiments, a disclosed expression cassette comprises a nucleotide sequence with at least about 70%, 75%, 80%, 85%, 90%, 95%, 98%, 99%, or 100% identity to the entire length of SEQ ID NO:7 or the complement thereof. In some embodiments, a disclosed expression cassette comprises a nucleotide sequence with at least about 70%, 75%, 80%, 85%, 90%, 95%, 98%, 99%, or 100% identity to the entire length of SEQ ID NO:8 or the complement thereof.

In some embodiments, a disclosed expression cassette comprises a nucleotide sequence with about 70%, 75%, 80%, 85%, 90%, 95%, 98%, 99%, or 100% identity to the entire length of SEQ ID NO:7 or the complement thereof. In some embodiments, a disclosed expression cassette comprises a nucleotide sequence with about 70%, 75%, 80%, 85%, 90%, 95%, 98%, 99%, or 100% identity to the entire length of SEQ ID NO:8 or the complement thereof.

In some embodiments, a disclosed expression cassette comprises a nucleotide sequence consisting of 100% identity to the entire length of SEQ ID NO:7 or the complement thereof. In some embodiments, a disclosed expression cassette comprises a nucleotide sequence consisting of 100% identity to the entire length of SEQ ID NO:8 or the complement thereof.

In some embodiments, a disclosed expression cassette is stably integrated into the genome of a host organism. In some embodiments, a disclosed expression cassette is stably integrated into the genome of a microbe, such as a fungal cell. In some embodiments, a disclosed expression cassette is stably integrated into the genome of a species of Pichia, e.g., Pichia pastoris, a species of Saccharomyces, e.g., Saccharomyces cerevisiae, including strains FL100 or S288C, or a species of Yarrowia, e.g., Yarrowia lipolytica. In some embodiments, a disclosed expression cassette is integrated, such as stably integrated, into the genome of a host organism by transposition, such as a transposon-mediated method of genome integration.

In some embodiments, a transposon system is used to integrate a disclosed expression cassette into the genome of a host organism. In some embodiments, the transposon system comprises a transposon vector and a transposase. In some embodiments, the transposon system is piggyBac, Super piggyBac, Sleeping Beauty, Hyperactive Sleeping Beauty, a helitron transposon system, a Tol2 transposon system, a TcBuster transposon system, including like, e.g., piggyBac-like, or mutant transposon systems thereof.

In some embodiments, disclosed expression cassettes are integrated into the genome of a host cell by a transposase comprising a nucleotide sequence with at least 70%, 75%, 80%, 85%, 90%, 95%, or 100% identity to the entire length of SEQ ID NO:10. In some embodiments, disclosed expression cassettes are integrated into the genome of a host cell by a transposase comprising a nucleotide sequence with about 70%, 75%, 80%, 85%, 90%, 95%, 97%, 98%, 99%, or 100% identity to the entire length of SEQ ID NO:10.

In some embodiments, disclosed expression cassettes are integrated into at least one site, such as an integration site or site of integration, in the genome of the host organism. In some embodiments, disclosed expression cassettes are integrated into at least two, three, four, five, six, seven, eight, nine, or ten sites in the host organism's genome. In some embodiments, disclosed expression cassettes are integrated into one, two, three, four, five, six, seven, eight, nine, or ten sites of integration in the host cell genome.

In some embodiments, a disclosed expression cassette is integrated into the genome of host organism is S. cerevisiae, such as strain FL100, at any one or more of the integration sites chromosome IV (NC_001136 at position 543,705); chromosome IV (NC_001136 position 1,357,520); chromosome VIII (NC_001140 position 124,029); chromosome X (NC_001142 position 181,309); chromosome XI (NC_001143 position 300,654); and chromosome XII (NC_001144 position 213,991).

### Host Organisms

In some aspects, provided herein are host organisms comprising a polynucleotide sequence, such as an expression cassette, encoding an interfering RNA molecule which is capable of inhibiting the expression of a gene in a mosquito, flanked by an inverted terminal repeat sequence, such as a host organism comprising a disclosed expression cassette.

In some embodiments, a disclosed host organism is a prototroph, such as a prototrophic yeast cell. In some embodiments, the disclosed host organism is a prototroph of Pichia, Saccharomyces, or Yarrowia. In some embodiments, the disclosed host organism is a prototroph of Pichia pastoris, Saccharomyces cerevisiae, or Yarrowia lipolytica. In some embodiments, the disclosed host organism is a prototrophic Saccharomyces cerevisiae strain FL100. In some embodiments, the disclosed host organism is a prototrophic Saccharomyces cerevisiae strain S288C.

In some embodiments, disclosed host organisms comprise the auxotrophic rescue promoter flanked by a 5′ inverted terminal repeat. In some embodiments, disclosed host organisms comprise the nucleotide sequence encoding the interfering RNA molecule flanked by a 3′ inverted terminal repeat. In some embodiments, the expression cassette is integrated, such as stably integrated, into the genome of the host cell.

In some embodiments, a disclosed host organism comprises a nucleotide sequence encoding an interfering RNA molecule, wherein the interfering RNA molecule is an RNA construct, a double stranded RNA (dsRNA), a small interfering RNA (siRNA), a short hairpin RNA (shRNA), or an anti-sense oligonucleotide.

In some embodiments, the disclosed host organism produces an interfering RNA molecule that inhibits expression of a gene in a mosquito that is a species of Aedes, Anopheles, or Culex. In some embodiments, the disclosed host organism produces an interfering RNA molecule that inhibits expression of a gene in Anopheles gambiae, Aedes aegypti, Aedes albopictus, Culex pipiens, or Culex quinquesfasciatus. In some embodiments, the disclosed host organism produces an interfering RNA molecule that inhibits the expression of Shaker in an Aedes spp., Anopheles spp., or Culex spp., e.g., Anopheles gambiae, Aedes aegypti, Aedes albopictus, Culex pipiens, or Culex quinquesfasciatus.

In some embodiments, the expression cassette is stably integrated into the genome of the host organism. In some embodiments, the disclosed host organism is a microbe, such as a fungal cell, such as a yeast cell. In some embodiments, the disclosed host organism is a species of Pichia, e.g., Pichia pastoris, a species of Saccharomyces, e.g., Saccharomyces cerevisiae, including strains FL100 or S288C, or a species of Yarrowia, e.g., Yarrowia lipolytica. In some embodiments, the disclosed host organism is Saccharomyces cerevisiae. In some embodiments, the disclosed host organism is Saccharomyces cerevisiae strain FL100 or Saccharomyces cerevisiae strain S288C.

Saccharomyces cerevisiae strain FL100 has been described by Casaregola et al., Yeast 1998; 14(6):551-64 and the whole genome shotgun sequence is available at GenBank Accession No: JRIT00000000.1. Additionally, the S. cerevisiae S288C genome has been described by Fisk et al., Yeast. 2006 September; 23(12):857-65 and YeastGenome.org (GenBank Accession No.: GCF_000146045.2). General genomic details of disclosed host organisms are accessible to one of skill in the art.

In some embodiments, disclosed host organisms comprise an expression cassette comprising inverted terminal repeat sequences which facilitate integration of the expression cassette by a transposon system, such as by transposition. In some embodiments, the transposon system is piggyBac, Super piggyBac, Sleeping Beauty, Hyperactive Sleeping Beauty, a helitron transposon system, a Tol2 transposon system, a TcBuster transposon system, including like, e.g., piggyBac-like, or mutant transposon systems thereof.

In some embodiments, disclosed host organisms comprise an expression cassette, comprising a nucleotide sequence encoding an interfering RNA molecule, an auxotrophic rescue promoter, and an auxotrophic rescue gene wherein the auxotrophic rescue promoter is flanked by a 5′ inverted terminal repeat and the nucleotide sequence encoding the interfering RNA molecule is flanked by a 3′ inverted terminal repeat.

In some embodiments, disclosed host organisms have at least one site of genomic integration of the expression cassette, such as a disclosed expression cassette, in their genome. In some embodiments, disclosed host organisms have at least two, three, four, five, six, seven, eight, nine, or ten sites of genomic integration of the expression cassette in their genome. In some embodiments, disclosed host organisms have one, two, three, four, five, six, seven, eight, nine, or ten sites of integration of the expression cassette in their genome.

In some embodiments, disclosed host organisms have at least one site of genomic integration of a nucleotide sequence encoding the interfering RNA molecule in their genome. In some embodiments, disclosed host organisms have at least two, three, four, five, six, seven, eight, nine, or ten sites of genomic integration of a nucleotide sequence encoding the interfering RNA molecule in their genome. In some embodiments, disclosed host organisms have one, two, three, four, five, six, seven, eight, nine, or ten sites of integration of the nucleotide sequence encoding the interfering RNA molecule in their genome. In some embodiments, the interfering RNA molecule inhibits the expression of Shaker in a mosquito.

In some embodiments, disclosed host organisms comprise a nucleotide sequence encoding an interfering RNA molecule that targets the gene Shaker in the mosquito. In some embodiments, disclosed host organisms comprise a nucleotide sequence transcribed from the nucleotide sequence encoding the interfering RNA molecule which is partially or perfectly complementary to a sequence comprising at least 80%, 84%, 88%, 92%, 96%, or 100% identity to the entire length of SEQ ID NO: 2. In some embodiments, disclosed host organisms comprise an interfering RNA molecule which comprises at least 25 contiguous nucleotides which are partially or perfectly complementary to the entire length of SEQ ID NO: 2. In some embodiments, the nucleotide sequence encoding the interfering RNA molecule is integrated, such as stably integrated, into the genome of the host organism.

In some embodiments, a disclosed host organism comprises a nucleotide sequence encoding the interfering RNA molecule shRNA.463, which is represented by SEQ ID NO:1, wherein the nucleotide sequence is flanked by an inverted terminal repeat sequence. In some embodiments, a disclosed host organism comprises a nucleotide sequence that has at least 80% or at least 90% identity to SEQ ID NO:1, wherein the nucleotide sequence is flanked by an inverted terminal repeat sequence. In some embodiments, a disclosed host organism comprises a nucleotide sequence that has 80%, 82%, 84%, 86%, 88%, 90%, 92%, 94%, 96%, or 98% to SEQ ID NO:1, wherein the nucleotide sequence is flanked by an inverted terminal repeat sequence. In some embodiments, a disclosed host organism comprises a nucleotide sequence that has 100% identity to the entire length of SEQ ID NO:1, wherein the nucleotide sequence is flanked by an inverted terminal repeat sequence. In some embodiments, a disclosed host organism comprises a nucleotide sequence encoding an interfering RNA molecule that consists of 100% identity to the entire length of SEQ ID NO:1, wherein the nucleotide sequence is flanked by an inverted terminal repeat sequence. In some embodiments, the inverted terminal repeat sequence flanks the 3′ end of the nucleotide sequence encoding the interfering RNA molecule.

In some embodiments, disclosed host organisms comprise a nucleotide sequence which has at least 80% or at least 90% sequence similarity to the entire length of SEQ ID NO. 1, wherein the nucleotide sequence is flanked by an inverted terminal repeat sequence. In some embodiments, a disclosed host organism comprises a nucleotide sequence that has 80%, 82%, 84%, 86%, 88%, 90%, 92%, 94%, 96%, or 98% to SEQ ID NO:1, wherein the nucleotide sequence is flanked by an inverted terminal repeat sequence. In some embodiments, disclosed host organisms comprise a nucleotide sequence comprising the entire length of SEQ ID NO. 1, wherein the nucleotide sequence is flanked by an inverted terminal repeat sequence. In some embodiments, disclosed host organisms comprise a nucleotide sequence consisting of the entire length of SEQ ID NO. 1, wherein the nucleotide sequence is flanked by an inverted terminal repeat sequence. In some embodiments, the inverted terminal repeat sequence flanks the 3′ end of the nucleotide sequence encoding the interfering RNA molecule.

In some embodiments, disclosed host organisms comprise an expression cassette comprising a nucleotide sequence with at least about 80%, 85%, 90%, 95%, 98%, or 99% sequence identity to the entire length of SEQ ID NO:7 or the complement thereof. In some embodiments, disclosed host organisms comprise an expression cassette comprising a nucleotide sequence with at least about 80%, 85%, 90%, 95%, 98%, or 99% sequence identity to the entire length of SEQ ID NO:8 or the complement thereof.

In some embodiments, a disclosed host organism is a yeast cell comprising the genotype MATa, PiggyBac (leu2d/P_{TDH3}-shRNA 463-T_{CYC1}), CEN/ARS (URA3/SPBase_Sc-CO).

In some embodiments, a disclosed host organism is yeast cell comprising the genotype MATa, PiggyBac (leu2d/P_{TDH3}-shRNA 463-T_{CYC1}, P_{TDH3}-shRNA 463-T_{CYC1}, P_{TDH3}-shRNA 463-T_{CYC1}), CEN/ARS (URA3/SPBase-Sc-CO).

In some embodiments, disclosed host organisms comprise an expression cassette comprising a minimal or truncated promoter operably linked to a selection marker gene, such as leu2d. In some embodiments, disclosed host organisms comprising the minimal or truncated promoter have increased expression levels of interfering RNA molecules relative to an expression cassette comprising a native or full-length promoter operably linked to the selection marker gene. In some embodiments, disclosed host organisms comprise expression levels of the interfering RNA molecules are increased by at least 25%, 50%, 75%, 100%, 125%, 150%, 175%, 200%, 225%, 250%, 275%, 300%, 325%, 350%, 375%, 400%, 425%, 450%, 475%, or 500% relative to an expression cassette comprising a native promoter operably linked to the selection marker gene. In some embodiments, expression levels of the interfering RNA molecules are increased by about 25%, 50%, 75%, 100%, 125%, 150%, 175%, 200%, 225%, 250%, 275%, 300%, 325%, 350%, 375%, 400%, 425%, 450%, 475%, or 500% relative to an expression cassette comprising a native promoter operably linked to the selection marker gene. In some embodiments, expression levels are 5 times greater, 10 times greater, 15 times greater, 20 times greater, 25 times greater, 30 times greater, 35 times greater, 40 times greater, 45 times greater, or 50 times greater relative to an expression cassette comprising a native promoter operably linked to the selection marker gene.

### Production of Interfering RNA Biopesticides

In some aspects, provided herein are methods of cultivating disclosed host organisms, such as interfering RNA biopesticides, at an industrial scale. In some embodiments, disclosed methods comprise cultivating a disclosed host organism in a fermentation media, such as a high cell-density fermentation media.

In some embodiments, growth of a disclosed host organism for about 72 hours in a fermentation media yields at least 5 g/L, 10 g/L, 15 g/L, 20 g/L, 25 g/L, 30 g/L, 35 g/L, 40 g/L, 45 g/L, or 50 g/L of dry cell weight. In some embodiments, growth of a disclosed host organism for about 72 hours in a fermentation media yields about 0 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, or 100 g/L, of dry cell weight. In some embodiments, the fermentation media is a high cell-density fermentation media.

In some embodiments, growth of a disclosed host organism for about 72 hours in a fermentation media reaches an OD600 of at least about 5, 25, 50, 75, or 100. In some embodiments, growth of a disclosed host organism for about 72 hours in a fermentation media reaches an OD600 of about 5, 10, 15, 20, 25, 30, 35, 40, 45, 50, 55, 60, 65, 70, 75, 80, 85, 90, 95, or 100. In some embodiments, the fermentation media is a high cell-density fermentation media.

In some embodiments, cultivating a host organism according to the methods described herein results in production, such as continuous production, of interfering RNA molecules. In some embodiments, cultivating a host organism in a fermentation media for about 72 hours results in at least about a 25%, 50%, 75%, 100%, 125%, 150%, 175%, 200%, 225%, 250%, 275%, 300%, 325%, 350%, 375%, 400%, 425%, 450%, 475%, or 500% increase in the expression level of the interfering RNA molecule relative to time=0 h. In some embodiments, cultivating a host organism in a fermentation media for about 72 hours results in about a 25%, 50%, 75%, 100%, 125%, 150%, 175%, 200%, 225%, 250%, 275%, 300%, 325%, 350%, 375%, 400%, 425%, 450%, 475%, or 500% increase in the expression level of the interfering RNA molecule relative to time=0 h. In some embodiments, the host organism is a yeast cell, such as P. pastoris, S. cerevisiae, or Y. lipolytica. In some embodiments, disclosed methods comprise cultivating the host organism in a high cell-density fermentation media. In some embodiments, the interfering RNA molecules are used as biopesticides to undermine the fitness and/or the survival of mosquitoes, such as in the control of mosquito populations.

In some examples, the fermentation media, such as a high cell-density fermentation media, is a proprietary or commercial composition including, e.g., a carbon source, such as glucose, a nitrogen source, such as peptone, and amino acids essential for the growth of yeasts. Methods of yeast cultivation, such as high-cell-density fed-batch cultivation, are described in, e.g., Hoek et al., Biotechnol Bioeng. 2000 Jun. 5; 68(5):517-23 and Vogel & Todaro, Cross-Flow Filtration. In Fermentation and Biochemical Engineering Handbook: Principles, process design and equipment. Elsevier Science. 1996; (7): 271-347.

### Compositions

In some aspects, provided herein are compositions comprising a disclosed polynucleotide, expression cassette, host organism, or a combination thereof. In some embodiments, disclosed compositions comprise a live host organism as disclosed herein. In some embodiments, disclosed compositions comprise a dead host organism as disclosed herein, such as a heat-killed and/or lyophilized interfering RNA biopesticide. In preferred embodiments, the host organism, such as the interfering RNA biopesticide, is heat inactivated and/or lyophilized to reduce or eliminate the ability of the host organism to grow once released into a treatment area. In preferred embodiments, a disclosed yeast interfering RNA biopesticide is heat inactivated to reduce or eliminate the ability of the yeast to grow once released into a treatment area. In some embodiments, the yeast is formulated in a ready-to use dry formulation. In some embodiments, the yeast is S. cerevisiae.

In some embodiments, disclosed compositions comprise an expression cassette which comprises a nucleotide sequence encoding an interfering RNA molecule, wherein the interfering RNA molecule is an RNA construct, a double stranded RNA (dsRNA), a small interfering RNA (siRNA), a short hairpin RNA (shRNA), or an anti-sense oligonucleotide. In some embodiments, the expression cassette is integrated, such as stably integrated into the genome of a host organism, e.g., a yeast interfering RNA biopesticide.

In some embodiments, disclosed compositions comprise an expression cassette which encodes an interfering RNA molecule that inhibits the expression of a gene in a mosquito that is a species of Aedes, Anopheles, or Culex. In some embodiments, disclosed compositions comprise an expression cassette which encodes an interfering RNA molecule that inhibits the expression of a gene in Anopheles gambiae, Aedes aegypti, Aedes albopictus, Culex pipiens, or Culex quinquesfasciatus. In some embodiments, the disclosed compositions comprise an expression cassette which encodes an interfering RNA molecule that inhibits the expression of Shaker in an Aedes spp., Anopheles spp., or Culex spp., e.g., Anopheles gambiae, Aedes aegypti, Aedes albopictus, Culex pipiens, or Culex quinquesfasciatus.

In some embodiments, disclosed compositions comprise an expression cassette comprising a nucleotide sequence which encodes an interfering RNA molecule that targets the gene Shaker in the mosquito. In some embodiments, the interfering RNA molecule is capable of inhibiting the expression of Shaker in the mosquito.

In some embodiments, disclosed compositions comprise a nucleotide sequence transcribed from the nucleotide sequence encoding the interfering RNA molecule which is partially or perfectly complementary to a sequence comprising at least 80%, 84%, 88%, 92%, 96%, or 100% identity to the entire length of SEQ ID NO: 2. In some embodiments, disclosed compositions comprise an expression cassette which encodes an interfering RNA molecule comprising at least 25 contiguous nucleotides which are partially or perfectly complementary to the entire length of SEQ ID NO: 2. In some embodiments, disclosed compositions comprise a disclosed expression cassette integrated into the genome of a host organism. In some embodiments, disclosed compositions comprise the nucleotide sequence encoding the interfering RNA molecule integrated, such as stably integrated, into the genome of the host organism.

In some embodiments, disclosed compositions comprise an expression cassette comprising a nucleotide sequence encoding the interfering RNA molecule shRNA.463, which is represented by SEQ ID NO:1, wherein the nucleotide sequence is flanked by a 3′ inverted terminal repeat sequence. In some embodiments, disclosed compositions comprise an expression cassette comprising a nucleotide sequence that has at least 80% or at least 90% identity to SEQ ID NO: 1, wherein the nucleotide sequence is flanked by an inverted terminal repeat sequence. In some embodiments, disclosed compositions comprise an expression cassette comprising a nucleotide sequence that 80%, 82%, 84%, 86%, 88%, 90%, 92%, 94%, 96%, or 98% to SEQ ID NO:1, wherein the nucleotide sequence is flanked by an inverted terminal repeat sequence. In some embodiments, disclosed compositions comprise an expression cassette comprising a nucleotide sequence that has 100% identity to the entire length of SEQ ID NO: 1, wherein the nucleotide sequence is flanked by an inverted terminal repeat sequence. In some embodiments, disclosed compositions comprise an expression cassette comprising a nucleotide sequence encoding an interfering RNA molecule that consists of 100% identity to the entire length of SEQ ID NO: 1, wherein the nucleotide sequence is flanked by an inverted terminal repeat sequence. In some embodiments, the inverted terminal repeat sequence flanks the 3′ end of the nucleotide sequence encoding the interfering RNA molecule.

In some embodiments, disclosed compositions comprise the nucleotide sequence encoding the interfering RNA molecule integrated into the genome of a host organism. In some embodiments, disclosed compositions comprise the nucleotide sequence encoding the interfering RNA molecule integrated, such as stably integrated, into the genome of the host organism.

In some embodiments, disclosed compositions comprise a disclosed expression cassette stably integrated into the genome of a microbe, such as a fungal cell. In some embodiments, disclosed compositions comprise a disclosed expression cassette stably integrated into the genome of a species of Pichia, e.g., Pichia pastoris, a species of Saccharomyces, e.g., Saccharomyces cerevisiae, a species of Yarrowia, e.g., Yarrowia lipolytica. In some embodiments, disclosed compositions comprise a disclosed expression cassette stably integrated into the genome of Saccharomyces cerevisiae strain FL100 or Saccharomyces cerevisiae strain S288C.

In some embodiments, a disclosed composition comprises a prototrophic yeast cell comprising the genotype MATa, PiggyBac (leu2dPTDH3-shRNA 463-TCYC1), CEN/ARS (URA3/SPBase_Sc-CO).

In some embodiments, a disclosed composition comprises prototrophic yeast cell comprising the genotype MATa, PiggyBac (leu2d/PTDH3-shRNA 463-TCYC1, PTDH3-shRNA 463-TCYC1, PTDH3-shRNA 463-TCYC1), CEN/ARS (URA3/SPBase-Sc-CO).

In some embodiments, disclosed compositions are insecticidal, such as mosquitocidal. In some embodiments, disclosed compositions undermine the fitness and/or the survival of a mosquito after being consumed by the mosquito. In some embodiments, disclosed compositions kill a mosquito after being consumed by the mosquito, such as a larval mosquito or an adult mosquito.

In some embodiments, disclosed compositions further comprise an insecticide, such as a chemical insecticide. In some embodiments, the insecticide is DEET (N,N-diethyl-meta-toluamidedeltamethrin), etofenprox, methoprene, permethrin, piperonyl butoxide, phenothrin, malathion, pyriproxyfen or a combination thereof.

In some embodiments, disclosed compositions comprise an attractant, such as a microbe, e.g., a bacteria cell or a fungal cell. In some embodiments, the attractant is a yeast. In some embodiments, the attractant is a species of Saccharomyces, such as S. cerevisiae. In some embodiments, disclosed compositions further comprise an additional attractant, such as a bait. In some embodiments, the additional attractant comprises sugar, an octanol, a plant extract, a pheromone, a volatile organic compound, carbon dioxide, lactic acid, ammonia, or a combination thereof. Mosquito attractants are described in, e.g., Dormont et al., J Chem Ecol. 2021 May; 47(4-5):351-393.

In some embodiments, disclosed compositions further a sugar bait. In some embodiments, the bait is present within a trap, such as a lure trap. In some embodiments, the bait and/or the trap comprises any of the nucleic acids, including expression cassettes, host organisms, such as interfering RNA biopesticides, or compositions disclosed herein.

In some embodiments, the bait comprises an attractant. In some embodiments, the attractant is sugar. In some embodiments, the bait is an attractive targeted sugar bait or an attractive toxic sugar bait (ATSB). Attractive targeted sugar baits typically contain an attractant, including a form of sugar, such as a fruit syrup, and a toxic agent, such as a chemical insecticide. See, e.g., Wongthangsiri et al., Agriculture and Natural Resources, 2018; 52(4):393-398.

Lure traps are commonly used to attract and kill insect pests. Design and use of such traps are well known to those of skill in the art. A lure trap of the invention can be any device into which the interfering RNA biopesticides and compositions thereof are placed, and that prevents the insect pest, such as a mosquito, from escaping once the insect pest has come into contact with the trap. The traps can be of various sizes, shapes, colors, and materials. Traps may be designed and manufactured specifically for use as an insect trap or can be a container converted and adapted from other uses, e.g., a glass Petri dish, a metal coffee can, a cardboard box, or any ordinary plastic, metal, fiberglass, composite or ceramic container.

### Methods of Use

In some aspects, provided herein are methods of using a disclosed polynucleotide, expression cassette, host organism, such as an interfering RNA biopesticide composition, or a combination thereof, such as in the control of mosquito populations. In some embodiments, disclosed methods involve contacting a mosquito with a disclosed polynucleotide, expression cassette, host organism, composition, or a combination thereof, such that contacting the mosquito leads to consumption by the mosquito.

In some embodiments, disclosed methods involve contacting mosquito larvae with a disclosed polynucleotide, expression cassette, host organism, composition, or a combination thereof. In some embodiments, disclosed methods involve contacting an adult mosquito with a disclosed polynucleotide, expression cassette, host organism, composition, or a combination thereof. Herein, reference to a mosquito generally includes reference to different stages of life, such as pupae, larvae, and adult. Additionally, herein, contacting a mosquito encompasses consumption of the disclosed polynucleotide, expression cassette, host organism, composition, or a combination thereof by the mosquito or feeding of the same to the mosquito.

In some embodiments, the host organism is a yeast cell. a species of Pichia, e.g., Pichia pastoris, a species of Saccharomyces, e.g., Saccharomyces cerevisiae, including strains FL100 or S288C, or a species of Yarrowia, e.g., Yarrowia lipolytica. In some embodiments, the host organism is Saccharomyces cerevisiae, such as strain FL100 or S288C.

In some embodiments, disclosed methods further comprise contacting the mosquito with an insecticide, such as a chemical insecticide. In some embodiments, the insecticide contacting the mosquito is DEET (N,N-diethyl-meta-toluamidedeltamethrin), etofenprox, methoprene, permethrin, piperonyl butoxide, phenothrin, malathion, pyriproxyfen or a combination thereof.

In some embodiments, disclosed methods further comprise contacting the mosquito with an additional attractant, such as a bait. In some embodiments, the additional attractant comprises sugar, an octanol, a plant extract, a pheromone, a volatile organic compound, carbon dioxide, lactic acid, ammonia, or a combination thereof. Mosquito attractants are described in, e.g., Dormont et al., J Chem Ecol. 2021 May; 47(4-5):351-393.

In some embodiments, the bait is a sugar bait, such as an attractive targeted sugar bait or an attractive toxic sugar bait (ATSB). In some embodiments, the sugar bait comprises sugar and at least one insecticide, such as a mosquitocidal pesticide.

ATSBs, which are known in the art and commercially available, may include a sugar bait and a toxic agent, e.g., an insecticide. ATSBs are described in, e.g., WO2020185583Δ1, WO2009150254Δ1, Hapairai et al., Insect Biochem Mol Biol. 2020 May; 120: 103359, Mysore et al., PLoS Negl Trop Dis. 2020 July; 14(7): e0008479, Wongthangsiri et al., Agric. Nat. Resour 2018; 52(4):393-398, Khan et al., PLoS One. 2013 Sep. 24; 8(9):e77225, Fraser et al., Malar J. 2021 Mar. 17; 20(1):15.

In some embodiments, the disclosed methods result in a percent (%) mortality of at least about 10% 50%, at least about 60%, at least about 70%, at least about 75%, at least about 80%, at least about 90%, at least about 95%, at least about 98%, or at least about 100% mortality, including any and all numerical values and ranges in between.

### EXAMPLES

### Example 1: Use of Cas-CLOVER and piggyBac in Combination to Generate Yeast Stably Integrated with an Expression Cassette Encoding Interfering RNA Pesticide Sh.463

S. cerevisiae was engineered to produce Sh.463 using a combination of the Cas-CLOVER and piggyBac systems. The yeast strains have multiple integrations of the Sh.463 shRNA expression cassette cargo, which is directed to interfering with expression of a conserved sequence in the mosquito Shaker gene. Previously, the efficacy of the dual action larvicidal and adulticidal interfering RNA pesticide Sh.463 was described (Mysore et al., PLoS Negl Trop Dis., 14(7): e0008479), with production and delivery of Sh.463 achieved by engineering S. cerevisiae to express the RNA molecule. Prior studies involved synthesis of custom DNA oligonucleotides, which encode an shRNA expression cassette corresponding to the Sh.463 target sequence, stable transformation of S. cerevisiae according to previously described methods (Hapairai et al., Sci Rep. 2017; 7(1):13223).

### Methods

Yeast strain construction was performed in accordance with methods described by Brizzee et al., J Fungi 2023, 9, 1056. Briefly, to generate auxotrophic mutants, Cas-CLOVER was expressed under the ScRNR2 yeast promoter on a CEN/ARS plasmid with kanamycin resistance. The URA3 gene was targeted using a left URA3 gRNA (SEQ ID NO:17) and a right URA3 gRNA (SEQ ID NO:18) expressed by an SNR52 promoter and an SNR52 terminator. The guides and Cas-CLOVER were all on one plasmid and transformed into S. cerevisiae FL100 with a homologous donor repair (HDR) fragment that was 200 bp upstream of the start codon and 200 bp downstream of the stop codon of the URA3 gene.

After the strain was cured of the plasmid, and PCR verified that the full ORF was deleted, this strain was then used to target the LEU2 gene. Cas-CLOVER was expressed under the ScRNR2 yeast promoter on a CEN/ARS plasmid with hygromycin resistance. The LEU2 gene was targeted with a left LEU2 gRNA (SEQ ID NO:19) and a right LEU2 gRNA (SEQ ID NO:20) expressed by an SNR52 promoter and an SNR52 terminator. The guides and Cas-CLOVER were all on one plasmid and transformed into S. cerevisiae FL100 (ura3Δ0) with a homologous donor repair (HDR) fragment that was 200 bp upstream of the start codon and 200 bp downstream of the stop codon of LEU2 gene. The strain was cured of the plasmid carrying the gRNAs and Cas-CLOVER, and full ORF deletion of the leu2 gene was confirmed by PCR. The resultant genotype was MATa, ura3Δ0, leu2Δ0.

A Super piggyBac transposase was cloned onto a URA3 selection plasmid with a CEN/ARS replication of origin. The base piggyBac transposon plasmid was produced by cloning a minimal Leu2d promoter and Leu2 gene from pRS425, the plasmid map of which is accessible to one of skill in the art, e.g., via www.snapgene.com. A nucleotide sequence encoding the minimal Leu2d promoter and the Leu2 gene was inserted inside the piggyBac ITRs. The nucleotide sequence encoding Sh.463 shRNA (SEQ ID NO:1) was introduced by cloning the shRNA cassette, which included nucleotide sequences encoding a GAP promoter (SEQ ID NO:11), Sh.463 shRNA (SEQ ID NO:1), and a CYC1 terminator (SEQ ID NO:12) into a multi-cloning site inside of the piggyBac ITRs and upstream of the LEU2 selection marker.

S. cerevisiae were transformed with a 3:1 ratio of Transposon to transposase (750 ng:250 ng), per the instructions of the EZ-yeast transformation Kit. After incubation for one hour at 30° C., the Transformation solution was removed. Yeast were recovered in SD-URA and agitated overnight at 30° C. for piggyBac selection. On day 2, the cells were grown on a CM-Uracil agar plate for 2-3 days until colonies formed. The colonies were picked and placed on CM-leucine agar plates to select for transformants that incorporated the leucine selection marker. The colonies from this plate were then scaled into 96-well deep well plates for expansion.

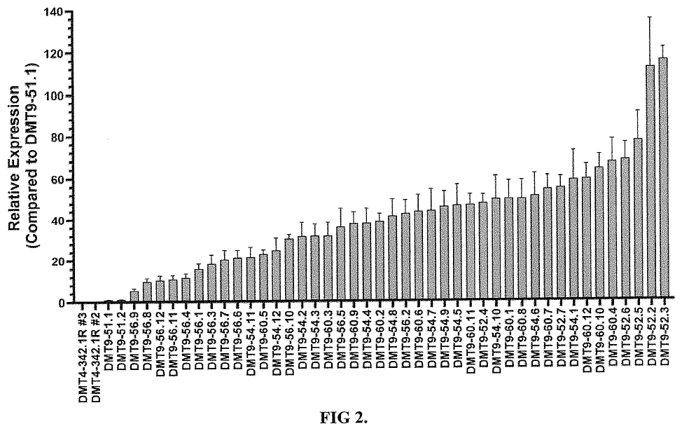

Sh.463 shRNA expression was verified via RNA extraction and cDNA synthesis, and levels of shRNA expression were assessed via qRT-PCR. FIG. 2 shows various strains expressing a range of mosquitocidal interfering RNA pesticide (IRP) levels. Relative expression was compared to that of control strain DMT9-51.1, which was engineered to express Sh.463 using only piggyBac integration.

Auxotrophies were restored, also referred to as auxotroph complementation, by amplifying 200 bp upstream or downstream of the ura3 gene or the leu2 gene from the parent S. cerevisiae FL100 strain and transformed in 1 μg of the nucleotide sequence encoding the minimal promoter leu2d and the leu2 gene, as described above with modifications. After one hour of incubation at 30° C., cells were recovered in YPD instead of selection media for 4 hours then plated on selection plates (CM-URA or CM-LEU2). Colonies were picked and put into seed plates with selection media then grown up for qPCR analysis.

### Example 2: Mosquito Larvicidal and Adulticidal ATSB Assays Involving Cas-CLOVER/piggyBac Yeast Strains Exhibiting Relatively High Expression of Sh.463

Yeast strains, which were engineered according to the methods in Example 1, exhibiting the greatest comparative levels of Sh.463 were selected for further study in mosquito-cidal assays. These strains were down-selected and their auxotrophies were restored (designated by ‘R’). Restoring, such as complementing, the auxotrophies for the transiently expressing yeast strain, DMT4-342 (pRS426_463, SEQ ID NO: 9), showed a 10-fold decrease in Sh.463 expression levels by qRT-PCR when compared to DMT9-51.1R, which is a low-expressing stably integrated piggyBac strain. The highest expressing strains, DMT9-52.2R #3 and DMT9-56.10R #3, were included in laboratory larvicidal studies conducted with Anopheles gambiae, Aedes aegypti, Culex quinquesfasciatus, and Culex pipiens.

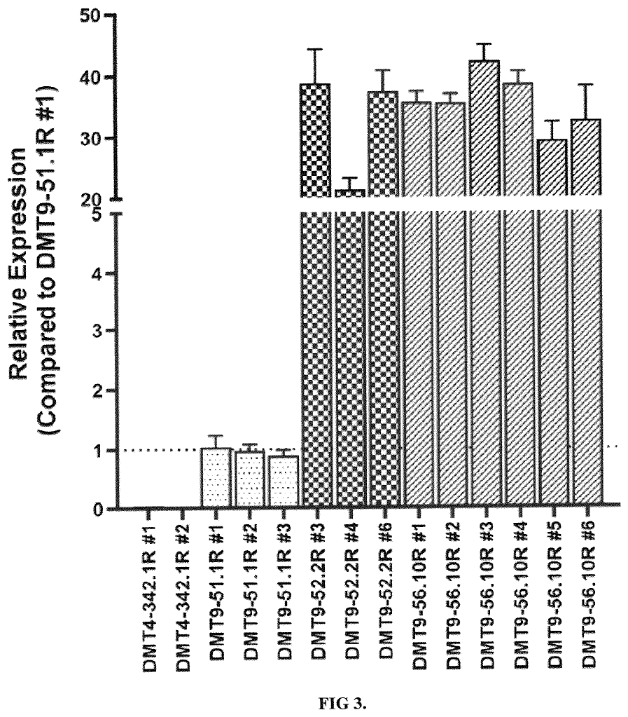

FIG. 3 shows down-selected and auxotrophy-re stored Cas-CLOVER/piggyac-synthesized yeast strains, which expressed relatively high levels of Sh.463. Sh.463 shRNA expression levels, which are shown relative to piggyfac integration strain DMT9-51.aR #1, were assessed via qRT-PCR, as described by Brizzee et al., J Fungi 2023, 9, 1056. The initial piggylac integration strain used a native leucine promoter (SEQ ID NO: 15) and had very low expression compared to subsequent strains that had minimal promoters and multiple copies of shRNA. Sh.463 genotypes and auxotrophy-restored genotypes are shown in Table 3.

TABLE 3
Genotypes of Down-Selected Yeast Strains

| shRNA | | | Original | |
|---|---|---|---|---|
| Type | Strain | Genotype | Auxotrophy | Restored Genotype |
| shRNA_463 | DMT4- | MATa, ura3Δ0, | Uracil, | MATa, (2 um |
| (Sh.463) | 342.1R | leu2Δ0, 2 um | Leucine | URA3/P_{TDH3}- |
| | | URA3/P_{TDH3}- | | shRNA_463-T_{CYC1}) |
| | | shRNA_463-T_{CYC1}) | | |
| | DMT9- | MATa, ura3Δ0, | Uracil | MATa, PiggyBac |
| | 51.1R | leu2Δ0, PiggyBac | | (LEU2/P_{TDH3}- |
| | | (LEU2/P_{TDH3}- | | shRNA_463-T_{CYC1}), |
| | | shRNA_463-T_{CYC1}), | | CEN/ARS |
| | | CEN/ARS | | (URA3/SPBase_Sc- |
| | | (URA3/SPBase_Sc- | | CO) |
| | | CO) | | |
| | DMT9- | MATa, ura3Δ0, | Uracil | MATa, PiggyBac |
| | 52.2R | leu2Δ0, PiggyBac | | (leu2d/P_{TDH3}- |
| | #3 | (leu2d/P_{TDH3}- | | shRNA_463-T_{CYC1}), |
| | | shRNA_463-T_{CYC1}), | | CEN/ARS |
| | | CEN/ARS | | (URA3/SPBase_Sc- |
| | | (URA3/SPBase_Sc- | | CO) |
| | | CO) | | |
| | DMT9- | MATa, ura3Δ0, | Uracil | MATa, PiggyBac |
| | 56.10R | leu2Δ0, PiggyBac | | (leu2d/P_{TDH3}- |
| | #3 | (leu2d/P_{TDH3}- | | shRNA_463-T_{CYC1}, |
| | | shRNA_463-T_{CYC1}, | | P_{TDH3}-shRNA_463- |
| | | P_{TDH3}-shRNA_463- | | T_{CYC1}, P_{TDH3}- |
| | | T_{CYC1}, P_{TDH3}- | | shRNA_463-T_{CYC1}), |
| | | shRNA_463-T_{CYC1}), | | CEN/ARS |
| | | CEN/ARS | | (URA3/SPBase-Sc- |
| | | (URA3/SPBase-Sc- | | CO) |
| | | CO) | | |
| Control | DMT4- | MATa, ura3D0, | Uracil | MATa, PiggyBac |
| | 347.1R | leu2D0, his3D0, | | (LEU2/PTDH3- |
| | | trp1D0, PiggyBac | | shRNA_Ctrl-TCYC1), |
| | | (LEU2/PTDH3- | | 2 um |
| | | shRNA_Ctrl-TCYC1), | | (URA3/SPBase_Sc- |
| | | 2 um | | CO), PiggyBac |
| | | (URA3/SPBase_Sc- | | (HIS3/PTDH3- |
| | | CO), PiggyBac | | shRNA_Ctrl-TCYC1), |
| | | (HIS3/PTDH3- | | CEN/ARS |
| | | shRNA_Ctrl-TCYC1), | | (URA3/SPBase_Sc- |
| | | CEN/ARS | | CO) PiggyBac |
| | | (URA3/SPBase_Sc- | | (trp1d/PTDH3- |
| | | CO) PiggyBac | | shRNA_Ctrl-TCYC1), |
| | | (trp1d/PTDH3- | | CEN/ARS |
| | | shRNA_Ctrl-TCYC1), | | (URA3/SPBase_Sc- |
| | | CEN/ARS | | CO) |
| | | (URA3/SPBase_Sc- | | |
| | | CO) | | |

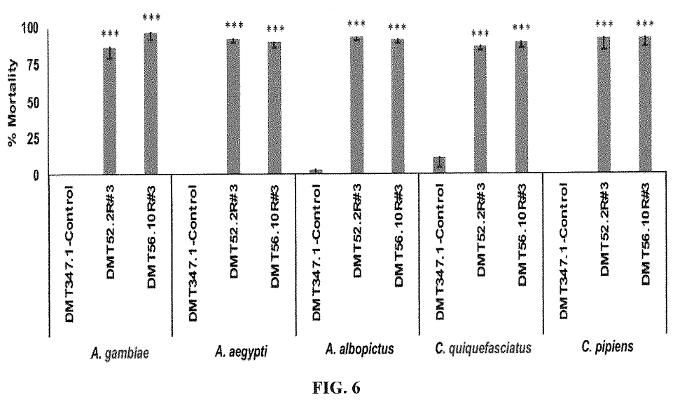

Larvicidal Assay: Larvicidal assays were conducted as previously described, with 40 mg of yeast per 20 larvae serving as a full dose treatment. See, e.g., Mysore et al., Insect genomics: Methods and protocols. 2019; 1858:213-231. Humana Press; Hapairai et al., Scientific Reports. 2017; 7:13223; Mysore et al., PLoS Negl Trop Dis., 2020; 14(7): e0008479. FIG. 6 shows larvicidal activity of heat-inactivated Cas-CLOVER/piggyBac-synthesized yeast strains expressing shRNA Sh.463 targeting the mosquito Shaker gene. Anopheles gambiae, Aedes aegypti, Culex quinquefasciatus, and Culex pipiens larvicide activity is shown for 50% doses of control S. cerevisiae strain DMT4-347.1R and S. cerevisiae Sh.463-expressing strains DMT9-52.2R #3 and DMT9-56.10R #3. DMT9-52.2R #3 and DMT9-56.10R #3 yeast treatments induced significant larval mortality with respect to control treatments (P<0.001).

These experiments demonstrated that dried heat-inactivated DMT9-52.2R #3 and DMT9-56.10R #3 strain yeast consumed by the larvae effectively killed mosquitoes prior to adulthood. Moreover, greater potency of the CLOVER/piggyBac Sh.463 yeast strains was observed compared to Sh.463 generated by conventional methods, as described in Mysore et al., PLoS Negl Trop Dis., 2020; 14(7): e0008479, Restated, lethal dosages were achieved using a reduced amount of the CLOVER/piggyBac Sh.463 yeast strains relative to those produced by the methods described in Mysore et al.

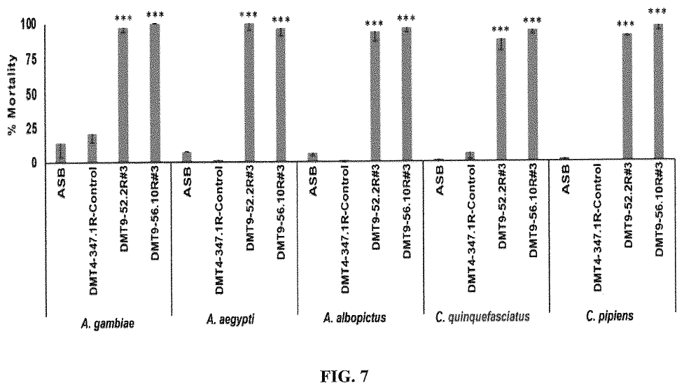

Adulticidal Assay: Attractive targeted sugar bait (ATSB) assays were conducted in accordance with previously described methods to assess the adulticidal activity of S. cerevisiae Sh.463-expressing strains DMT9-52.2R #3 and DMT9-56.10R #3. See, e.g., Mysore et al., Insects 2021; 12(11):986. Here, a full dose consists of ˜5 ul of 0.4 μg/μl yeast in the sugar bait. FIG. 7 shows adulticidal activity of Cas-CLOVER/piggyBac-synthesized yeast strains expressing shRNA Sh.463. Adult mosquito killing capacity was demonstrated using 50% strength of doses with DMT9-52.2R #3 and DMT9-56.10R #3 yeast delivered as an ATSB to adult female A. gambiae, A. aegypti, A. albopictus, C. quinquefasciatus, and C. pipiens mosquitoes. Both DMT9-52.2R #3, and DMT9-56.10R #3 induced significant adult female mortality with respect to the control yeast and sugar bait (ASB) treatments (P<0.001, ANOVA).

These trials, which were conducted on adult females, demonstrated the adulticidal capacity of the yeast strains generated using the Cas-CLOVER/piggyBac system. As with the larvicide assays, lethal dosages could be obtained with a smaller amount of yeast than had been used when similar experiments were performed with laboratory yeast strains in the past (unpublished data). These studies indicate not only the feasibility but also the advantages of using Cas-CLOVER and piggyBac to engineer S. cerevisiae containing stable integrations of the Sh.463 shRNA expression cassette and use of the same to control larvae and adult populations of Aedes, Anopheles, and Culex mosquitoes.

Following completion of the insecticidal trials for the two top yeast strains, the fully restored strains (see Table 3) were subsequently grown by shake culture on synthetic growth media and industrial fermentation media. The high growth rates achieved in these cultures suggested that the yeast will perform well in industrial-sized scale-up fermentation, enabling global deployment of these insecticides. Additional fermentation scale-up studies are described in Example 4.

### Example 3: Whole Genome Sequencing of Cas-CLOVER/piggyBac-Engineered Saccharomyces cerevisiae Strains Expressing Mosquito-Cidal shRNA Sh.463

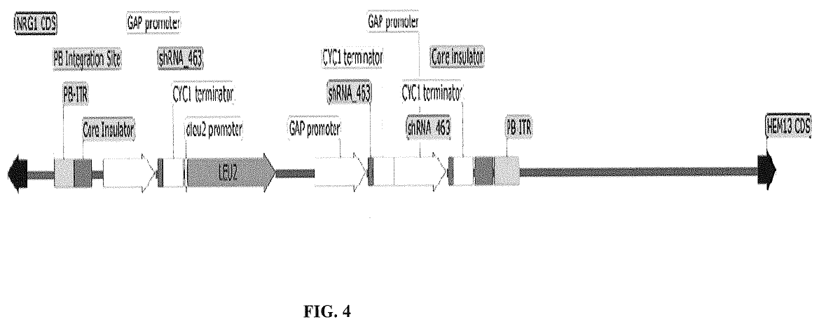

Whole Genome Sequencing was performed via Oxford Nanopore Technology and verified externally to determine the genomic integration sites of DMT9-52.2R #3 and DMT9-56.10R #3. For DMT9-56.10R #3, 135 sequencing reads were identified and mapped directly to the piggyBac transposon and the flanking sequences of the piggyBac ITRs were searched via BLASTn (https://blast.ncbi.nlm.nih.gov/Blast.cgi) to identify sites of genomic integration. These reads identified that the insecticidal cargo was integrated on chromosome IV (NC_001136) at position 543,705 which is between the genes, NRG1 and HEM13, as shown in FIG. 4.

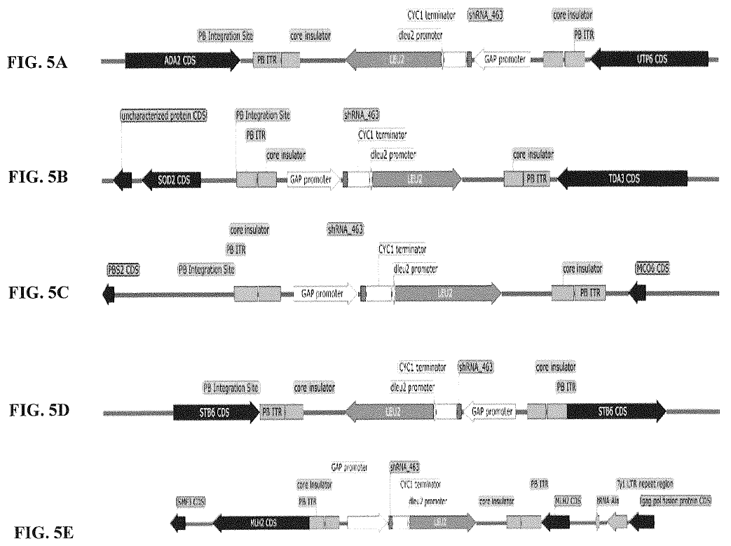

As for DMT9-52.2R #3, the sequencing revealed five different genomic integration sites: (A) chromosome IV (NC_001136 position 1,357,520) in between the genes ADA2 and UTP6, (B) VIII (NC_001140 position 124,029) in between the genes, SOD2 and TDA3, (C) X (NC_001142 position 181,309) in between the genes, PBS2 and MCO6, (D) XI (NC_001143 position 300,654) intragenic of STB6, and (E) XII (NC_001144 position 213,991) intragenic of MLH2, as shown in FIG. 5.

Accordingly, insecticidal Sh.463 shRNA expression cassette is stably integrated into chromosome IV, VIII, X, XI, and XII, and two out the five genomic integrations were intragenic and occurred in the STB6 and MLH2 gene of chromosome XI and XII, respectively. For each of these characterized yeast strains, the integration events, Sh.463 copy number, and genomic locations are summarized in Table 4.

TABLE 4
Summary of WGS data for yeast strains
DMT9-56.10R #3 and DMT9-52.2R #3

| | | | | | 5′ Flanking | 3′ Flanking |
|---|---|---|---|---|---|---|
| | Integra- | Sh.463 | Total | Genomic | Sequence | Sequence |
| Strain | tion(s) | Copies | Copies | Location | (60 bp) | (60 bp) |
| DMT9- | 5 | 1 | 5 | Chromosome | ATCGGTTCTT | AAGAATAAA |
| 52.2R | | | | IV | TCCAATTTTT | CATTTATCTG |
| #3 | | | | (1,357,520) | TTTTTTTTTT | ATATGTAATT |
| | | | | | TTTTTTTTTT | GCATTTATAA |
| | | | | | TTTTTTTTTT | AATGTACAGT |
| | | | | | GGTTGTAAT | ACCGCATTTA |
| | | | | | A (SEQ ID | A (SEQ ID |
| | | | | | NO: 22) | NO: 23) |
| | | | | Chromosome | AAGAAATAT | ATATATAAAT |
| | | | | VIII | ATAGATTTA | AAATAAGCTC |
| | | | | (124,029) | GGTATTCGT | TTATATGTAC |
| | | | | | TAAATATAT | AAATTTGTGC |
| | | | | | ACACATTAA | ATATACTTTT |
| | | | | | ATGGCCTCA | CTTGACCTTT |
| | | | | | GAAATT | (SEQ ID |
| | | | | | (SEQ ID | NO: 25) |
| | | | | | NO: 24) | |
| | | | | Chromosome | AGTCTAAGC | ATCGTTCTCG |
| | | | | X | TGAAAGATT | TGGACGAGAT |
| | | | | (181,309) | ATTACTTTCA | TAAAAATAG |
| | | | | | TTTGATTTTT | AAATGATGTA |
| | | | | | TTATTTTTGA | GAGGAGATG |
| | | | | | AGCCCCATT | CACTAAACAT |
| | | | | | TCC (SEQ ID | TG (SEQ ID |
| | | | | | NO: 26) | NO: 27) |
| | | | | Chromosome | TAACAATTG | AAAGGTTGTG |
| | | | | XI | ATAAATTAT | AAGTCAACTG |
| | | | | (300,654) | TTGAAGTAT | TTCAAGACAT |
| | | | | | CTTCCAAGA | GACTGGCAA |
| | | | | | CTTCAAACA | AGGAAACTTT |
| | | | | | AAGATATTT | ATGCATCTAT |
| | | | | | TCAAGT | C (SEQ ID |
| | | | | | (SEQ ID | NO: 29) |
| | | | | | NO: 28) | |
| | | | | Chromosome | TTTTTCTTGG | TATTACTGTA |
| | | | | XII | CCTCGAAGA | GTCCCCACGG |
| | | | | (213,991) | AATTTCGAG | GACAAGATA |
| | | | | | ATACCTTGC | CCTTGTACCT |
| | | | | | TCGTAACCC | TTTTCCATTG |
| | | | | | TCCCAGAAG | GTAATACCAC |
| | | | | | TTTCC (SEQ | C (SEQ ID |
| | | | | | ID NO: 30) | NO: 31) |
| DMT9- | 1 | 3 | 3 | Chromosome | GAATAACGG | AAGAAAAAA |
| 56.10R | | | | IV (543,705) | AAAAGGAGC | CACCCCAAAC |
| #3 | | | | | CTGCAGCCA | ACCCTGACCG |
| | | | | | GACTGTAGA | GCGGCGAAG |
| | | | | | AAGATGACA | CCCCTCTGCG |
| | | | | | CTGCCAAGA | CGCTCAACGC |
| | | | | | GAATAA | GT (SEQ ID |
| | | | | | (SEQ ID | NO: 33) |
| | | | | | NO: 32) | |

### Example 4: Fermentation Studies of Cas-CLOVER/piggyBac-Engineered Saccharomyces cerevisiae Strains Expressing Mosquito-Cidal shRNA Sh.463

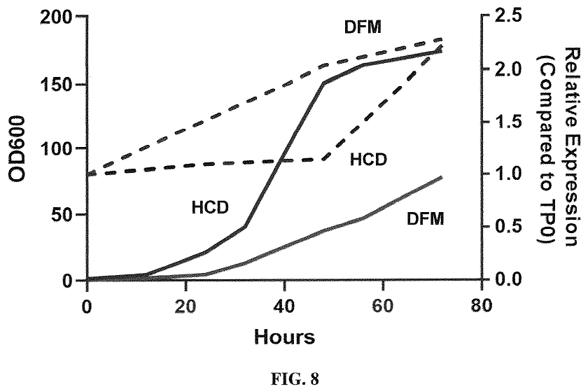

Pilot fermentation studies were performed in accordance with methods described by Brizzee et al., J. Fungi 2023, 9, 1056 to determine whether industrial-sized scale-up fermentation would be economically feasible. Confirmation of feasibility would bolster the added advantage of amenability to widespread cultivation and deployment of the engineered yeast. At the 5 L to 10 L fermentation scale, DMT9-56.10R #3 yielded 121.3 g/L and 23.5 g/L dry cell weight (DCW) using two different fermentation media for high-cell density growth, HCD and DFM, respectively. FIG. 8 shows DMT9-56.10R #3 72-hour growth curves and Sh.463 expression in the presence of HCD and DFM. Relative expression was quantified via qRT-PCR and was compared to expression at timepoint 0 (TP0). Dashed lines correspond to each media condition HCD and DFM. The optical densities for these two fermentation medias reached 173.92 and 77.81 over the course of 72 hours, respectively (FIG. 8, solid lines). In addition to the high growth conditions, the expression of Sh.463 stayed steady throughout the fermentation, regardless of the media (FIG. 8, dashed lines).

Fermentations at the scale of 5 L to 10 L fermentations were performed because the systematic parameters are scalable to large-scale fermenters, e.g., volumes in excess of >100 L. Furthermore, downstream processes are currently underway to optimize cleaning of the yeast material similar to that of nutritional yeast manufacturing. See, e.g., Vogel & Todaro, Fermentation and Biochemical Engineering Handbook: Principles, process design and equipment. Elsevier Science. 1996; 7:271-347.

### Example 5: Field Trials

Larvicide field assays are presently being conducted in Trinidad and Tobago. Guiyun Yan's laboratory has described several potential field sites in Kenya (Kweka et al., PLoS One. 2012; 7(12): e52084). Several field sites exist in the region adjacent to St. Augustine, Trinidad and Tobago. The site is located on an isolated stretch along Los Armadillos Road. The area around the site is completely forested for several kilometers. The Severson and Chadee labs have performed larval sampling experiments at these sites, where they have found an abundance of A. aegypti larvae.

Small Scale Field Trials: Small scale field trials are conducted on natural mosquitoes located in natural breeding sites. The objectives of these studies are to: i) determine the efficacy, including residual activity of interfering RNA larvicides in natural breeding sites, ii) identify the optimum field application dosage, iii) monitor abiotic parameters which may impact the efficacy of the larvicides, and to iv) record qualitative observations of non-target biota cohabitating with mosquito larvae (WHO, 2005). Specific interfering RNA larvicides and delivery strategies deemed to be suitable in our simulated field and semi-field experiments are assessed in the field. The habitats to be evaluated in these experiments include natural and man-made containers that are not used to store drinking water. As discussed in the WHO guidelines (2005), at least three replicates of each type of habitat are randomly selected for each dosage of the experimental or control larvicide formulation. Post-treatment immature (first and second instar, third and fourth instar, and pupal) abundance are monitored in samples taken just prior to treatment, on day 2, and then weekly until the density of fourth instar larvae in treated habitats is comparable to that of the control containers. Efficacy and residual activity are ascertained through measurement of the pre- and post-treatment abundance of each larval instar and pupae, accounting for the dynamics of change occurring in the treated and control containers. Adult emergence is monitored by sampling and counting pupal skins. Data are assessed as described in the WHO (2005) guidelines using ANOVA.

Large Scale Field Trials: The efficacy of larvicides deemed acceptable in small field trials is verified in larger scale field trials conducted according to the WHO (2005) guidelines on natural mosquito populations located in natural breeding habitats. The objectives of these trials include: i) confirmation of the efficacy of the larvicide at the selected field application dosage when applied to large-scale plots in natural breeding habitats, ii) verification of larvicide residual activity and application intervals, iii) examination of the ease of larvicide application and dispersal, iv) assessment of community acceptance of this intervention, and v) detection of any unanticipated effects of the treatment on non-targeted organisms (WHO, 2005). Pre-treatment densities of larvae and pupae are assessed in each larval habitat at least twice over the course of a week prior to treatments. The habitats to be evaluated include those assessed in the small scale field trials, but 25 replicates of each habitat are assessed for each control or experimental treatment. Samples are taken and assessed, and data is evaluated using the same general procedure described for the small scale trials. Nontarget organisms cohabitating with mosquito larvae are also be counted and examined to ascertain unanticipated impacts of the larvicide treatments. Furthermore, the ease of storage, handling, and application of the insecticide formulation are assessed. Observations on the acceptability of these larvicides to residents of the area will also be recorded.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Sequence Table

| SEQ | | |
|---|---|---|
| ID | | |
| NO. | Sequence | Description |
| 1 | gttaaattatctaggcattcgattcaagagatcgaatgcc | shRNA of |
| | tagataatttaa | siRNA.463 |
| 2 | auuuaaauuaucuaggcauucgaaa | Target |
| | | sequence |
| | | Shaker |
| | | (AAEL0002 |
| | | 42) |
| 3 | atatatatatttcaaggatataccattcta | Truncated |
| | | (defective) |
| | | leu2 |
| | | nutritional |
| | | promoter |
| 4 | ttaaccctagaaagataatcatattgtgacgtacgttaaa | 3′ piggyBac |
| | gataatcatgcgtaaaattgacgcatgtgttttatcggtc | Inverted |
| | tgtatatcgaggtttatttattaatttgaatagatattaa | Terminal |
| | gttttattatatttacacttacatactaataataaattca | Repeat |
| | acaaacaatttatttatgtttatttatttattaaaaaaaa | transposable |
| | acaaaaactcaaaatttcttctataaagtaacaaaacttt | element |
| | ta | |
| 5 | gatatctataacaagaaaatatatatataataagttatca | 5′ piggyBac |
| | cgtaagtagaacatgaaataacaatataattatcgtatga | Inverted |
| | gttaaatcttaaaagtcacgtaaaagataatcatgcgtca | Terminal |
| | ttttgactcacgcggtcgttatagttcaaaatcagtgaca | Repeat |
| | cttaccgcattgacaagcacgcctcacgggagctccaagc | transposable |
| | ggcgactgagatgtcctaaatgcacagcgacggattcgcg | element |
| | ctatttagaaagagagagcaatatttcaagaatgcatgcg | |
| | tcaattttacgcagactatctttctagggttaa | |
| 6 | gagggacagcccccccccaaagcccccagggatgtaatta | piggyBac |
| | cgtccctcccccgctagggggcagcagcgagccgcccggg | Core |
| | gctccgctccggtccggcgctccccccgcatccccgagcc | Insulators |
| | ggcagcgtgcggggacagcccgggcacggggaaggtggca | |
| | cgggatcgctttcctctgaacgcttctcgctgctctttga | |
| | gcctgcagacacctggggggatacggggaaaa | |
| 7 | ttaaccctagaaagataatcatattgtgacgtacgttaaa | piggyBac |
| | gataatcatgcgtaaaattgacgcatgtgttttatcggtc | Transposon |
| | tgtatatcgaggtttatttattaatttgaatagatattaa | carrying |
| | gttttattatatttacacttacatactaataataaattca | Sh.463 |
| | acaaacaatttatttatgtttatttatttattaaaaaaaa | expression |
| | acaaaaactcaaaatttcttctataaagtaacaaaacttt | cassette for |
| | tatagaattctatctagagggacagcccccccccaaagcc | DMT9- |
| | cccagggatgtaattacgtccctcccccgctagggggcag | 52.2R #3 |
| | cagcgagccgcccggggctccgctccggtccggcgctccc | |
| | cccgcatccccgagccggcagcgtgcggggacagcccggg | |
| | cacggggaaggtggcacgggatcgctttcctctgaacgct | |
| | tctcgctgctctttgagcctgcagacacctggggggatac | |
| | ggggaaaaggcctccaaggccagcttcccacaataagttg | |
| | ggtgaattttggctcattcctcctttctataggattgagg | |
| | tcagagctttgtgatgggaattctgtggaatgtgtgtcag | |
| | ttagggtgtggaaagtcccgcgatcgctcattatcaatac | |
| | tcgccatttcaaagaatacgtaaataattaatagtagtga | |
| | ttttcctaactttatttagtcaaaaaattagccttttaat | |
| | tctgctgtaacccgtacatgcccaaaataggggggggtta | |
| | cacagaatatataacatcgtaggtgtctgggtgaacagtt | |
| | tattcctggcatccactaaatataatggagcccgcttttt | |
| | aagctggcatccagaaaaaaaaagaatcccagcaccaaaa | |
| | tattgttttcttcaccaaccatcagttcataggtccattc | |
| | tcttagcgcaactacagagaacaggggcacaaacaggcaa | |
| | aaaacgggcacaacctcaatggagtgatgcaacctgcctg | |
| | gagtaaatgatgacacaaggcaattgacccacgcatgtat | |
| | ctatctcattttcttacaccttctattaccttctgctctc | |
| | tctgatttggaaaaagctgaaaaaaaaggttgaaaccagt | |
| | tccctgaaattattcccctacttgactaataagtatataa | |
| | agacggtaggtattgattgtaattctgtaaatctatttct | |
| | taaacttcttaaattctacttttatagttagtcttttttt | |
| | tagttttaaaacaccagaacttagtttcgacggattctag | |
| | aactagtggatccgttaaattatctaggcattcgattcaa | |
| | gagatcgaatgcctagataatttaattttttctcgagtca | |
| | tgtaattagttatgtcacgcttacattcacgccctccccc | |
| | cacatccgctctaaccgaaaaggaaggagttagacaacct | |
| | gaagtctaggtccctatttatttttttatagttatgttag | |
| | tattaagaacgttatttatatttcaaatttttcttttttt | |
| | tctgtacagacgcgtgtacgcatgtaacattatactgaaa | |
| | accttgcttgagaaggttttgggacgctcgaaggctttaa | |
| | tttgccggccggcctgcatatatatatttcaaggatatac | |
| | cattctaatgtctgcccctaagaagatcgtcgttttgcca | |
| | ggtgaccacgttggtcaagaaatcacagccgaagccatta | |
| | aggttcttaaagctatttctgatgttcgttccaatgtcaa | |
| | gttcgatttcgaaaatcatttaattggtggtgctgctatc | |
| | gatgctacaggtgttccacttccagatgaggcgctggaag | |
| | cctccaagaaggctgatgccgttttgttaggtgctgtggg | |
| | tggtcctaaatggggtaccggtagtgttagacctgaacaa | |
| | ggtttactaaaaatccgtaaagaacttcaattgtacgcca | |
| | acttaagaccatgtaactttgcatccgactctcttttaga | |
| | cttatctccaatcaagccacaatttgctaaaggtactgac | |
| | ttcgttgttgtcagagaattagtgggaggtatttactttg | |
| | gtaagagaaaggaagacgatggtgatggtgtcgcttggga | |
| | tagtgaacaatacaccgttccagaagtgcaaagaatcaca | |
| | agaatggccgctttcatggccctacaacatgagccaccat | |
| | tgcctatttggtccttggataaagctaatgttttggcctc | |
| | ttcaagattatggagaaaaactgtggaggaaaccatcaag | |
| | aacgaattccctacattgaaggttcaacatcaattgattg | |
| | attctgccgccatgatcctagttaagaacccaacccacct | |
| | aaatggtattataatcaccagcaacatgtttggtgatatc | |
| | atctccgatgaagcctccgttatcccaggttccttgggtt | |
| | tgttgccatctgcgtccttggcctctttgccagacaagaa | |
| | caccgcatttggtttgtacgaaccatgccacggttctgct | |
| | ccagatttgccaaagaataaggtcaaccctatcgccacta | |
| | tcttgtctgctgcaatgatgttgaaattgtcattgaactt | |
| | gcctgaagaaggtaaggccattgaagatgcagttaaaaag | |
| | gttttggatgcaggtatcagaactggtgatttaggtggtt | |
| | ccaacagtaccaccgaagtcggtgatgctgtcgccgaaga | |
| | agttaagaaaatccttgcttaaaaagattctcttttttta | |
| | tgatatttgtacataaactttataaatgaaattcataata | |
| | gaaacgacacgaaattacaaaatggaatatgttcataggg | |
| | tagacgaaactatatacgcaatctacatacatttatcaag | |
| | aaggagaaaaaggaggatgtaaaggaatacaggtaagcaa | |
| | attgatactaatggctcaacgtgataaggaaaaagaattg | |
| | cactttaacattaatattgacaaggaggagggcaccacac | |
| | aaaaagttaggtgtaacagaaaatcatgaaactatgattc | |
| | ctaatttatatattggaggattttctctaaaaaaaaaaaa | |
| | atacaacaaataaaaaacactcaatgacctgaccatttga | |
| | tggagtttaagtcaataccttccctgcaggtgtcctcaca | |
| | ggaacgaagtccctaaagaaacagtggcagccaggtttag | |
| | ccccggaattgactggattccttttttagggcccattggt | |
| | atggctttttccccgtatccccccaggtgtctgcaggctc | |
| | aaagagcagcgagaagcgttcagaggaaagcgatcccgtg | |
| | ccaccttccccgtgcccgggctgtccccgcacgctgccgg | |
| | ctcggggatgcggggggagcgccggaccggagcggagccc | |
| | cgggcggctcgctgctgccccctagcgggggagggacgta | |
| | attacatccctgggggctttgggggggggctgtcccttag | |
| | cggccgctgatatctataacaagaaaatatatatataata | |
| | agttatcacgtaagtagaacatgaaataacaatataatta | |
| | tcgtatgagttaaatcttaaaagtcacgtaaaagataatc | |
| | atgcgtcattttgactcacgcggtcgttatagttcaaaat | |
| | cagtgacacttaccgcattgacaagcacgcctcacgggag | |
| | ctccaagcggcgactgagatgtcctaaatgcacagcgacg | |
| | gattcgcgctatttagaaagagagagcaatatttcaagaa | |
| | tgcatgcgtcaattttacgcagactatctttctagggtta | |
| | a | |
| 8 | ttaaccctagaaagataatcatattgtgacgtacgttaaa | piggyBac |
| | gataatcatgcgtaaaattgacgcatgtgttttatcggtc | Transposon |
| | tgtatatcgaggtttatttattaatttgaatagatattaa | carrying |
| | gttttattatatttacacttacatactaataataaattca | Sh.463 |
| | acaaacaatttatttatgtttatttatttattaaaaaaaa | expression |
| | aaaaaactcaaaatttcttctataaagtaacaaaactttt | cassette for |
| | atagaattctatctagagggacagcccccccccaaagccc | DMT9- |
| | ccagggatgtaattacgtccctcccccgctagggggcagc | 56.10R #3 |
| | agcgagccgcccggggctccgctccggtccggcgctcccc | |
| | ccgcatccccgagccggcagcgtgcggggacagcccgggc | |
| | acggggaaggtggcacgggatcgctttcctctgaacgctt | |
| | ctcgctgctctttgagcctgcagacacctggggggatacg | |
| | gggaaaaggcctccaaggccagcttcccacaataagttgg | |
| | gtgaattttggctcattcctcctttctataggattgaggt | |
| | cagagctttgtgatgggaattctgtggaatgtgtgtcagt | |
| | tagggtgtggaaagtcccgcgatcgctcattatcaatact | |
| | cgccatttcaaagaatacgtaaataattaatagtagtgat | |
| | tttcctaactttatttagtcaaaaaattagccttttaatt | |
| | ctgctgtaacccgtacatgcccaaaataggggggggttac | |
| | acagaatatataacatcgtaggtgtctgggtgaacagttt | |
| | attcctggcatccactaaatataatggagcccgcttttta | |
| | agctggcatccagaaaaaaaaagaatcccagcaccaaaat | |
| | attgttttcttcaccaaccatcagttcataggtccattct | |
| | cttagcgcaactacagagaacaggggcacaaacaggcaaa | |
| | aaacgggcacaacctcaatggagtgatgcaacctgcctgg | |
| | agtaaatgatgacacaaggcaattgacccacgcatgtatc | |
| | tatctcattttcttacaccttctattaccttctgctctct | |
| | ctgatttggaaaaagctgaaaaaaaaggttgaaaccagtt | |
| | ccctgaaattattcccctacttgactaataagtatataaa | |
| | gacggtaggtattgattgtaattctgtaaatctatttctt | |
| | aaacttcttaaattctacttttatagttagtctttttttt | |
| | agttttaaaacaccagaacttagtttcgacggattctaga | |
| | actagtggatccgttaaattatctaggcattcgattcaag | |
| | agatcgaatgcctagataatttaattttttctcgagtcat | |
| | gtaattagttatgtcacgcttacattcacgccctcccccc | |
| | acatccgctctaaccgaaaaggaaggagttagacaacctg | |
| | aagtctaggtccctatttatttttttatagttatgttagt | |
| | attaagaacgttatttatatttcaaatttttctttttttt | |
| | ctgtacagacgcgtgtacgcatgtaacattatactgaaaa | |
| | ccttgcttgagaaggttttgggacgctcgaaggctttaat | |
| | ttgccggccggcctgcatatatatatttcaaggatatacc | |
| | attctaatgtctgcccctaagaagatcgtcgttttgccag | |
| | gtgaccacgttggtcaagaaatcacagccgaagccattaa | |
| | ggttcttaaagctatttctgatgttcgttccaatgtcaag | |
| | ttcgatttcgaaaatcatttaattggtggtgctgctatcg | |
| | atgctacaggtgttccacttccagatgaggcgctggaagc | |
| | ctccaagaaggctgatgccgttttgttaggtgctgtgggt | |
| | ggtcctaaatggggtaccggtagtgttagacctgaacaag | |
| | gtttactaaaaatccgtaaagaacttcaattgtacgccaa | |
| | cttaagaccatgtaactttgcatccgactctcttttagac | |
| | ttatctccaatcaagccacaatttgctaaaggtactgact | |
| | tcgttgttgtcagagaattagtgggaggtatttactttgg | |
| | taagagaaaggaagacgatggtgatggtgtcgcttgggat | |
| | agtgaacaatacaccgttccagaagtgcaaagaatcacaa | |
| | gaatggccgctttcatggccctacaacatgagccaccatt | |
| | gcctatttggtccttggataaagctaatgttttggcctct | |
| | tcaagattatggagaaaaactgtggaggaaaccatcaaga | |
| | acgaattccctacattgaaggttcaacatcaattgattga | |
| | ttctgccgccatgatcctagttaagaacccaacccaccta | |
| | aatggtattataatcaccagcaacatgtttggtgatatca | |
| | tctccgatgaagcctccgttatcccaggttccttgggttt | |
| | gttgccatctgcgtccttggcctctttgccagacaagaac | |
| | accgcatttggtttgtacgaaccatgccacggttctgctc | |
| | cagatttgccaaagaataaggtcaaccctatcgccactat | |
| | cttgtctgctgcaatgatgttgaaattgtcattgaacttg | |
| | cctgaagaaggtaaggccattgaagatgcagttaaaaagg | |
| | ttttggatgcaggtatcagaactggtgatttaggtggttc | |
| | caacagtaccaccgaagtcggtgatgctgtcgccgaagaa | |
| | gttaagaaaatccttgcttaaaaagattctctttttttat | |
| | gatatttgtacataaactttataaatgaaattcataatag | |
| | aaacgacacgaaattacaaaatggaatatgttcatagggt | |
| | agacgaaactatatacgcaatctacatacatttatcaaga | |
| | aggagaaaaaggaggatgtaaaggaatacaggtaagcaaa | |
| | ttgatactaatggctcaacgtgataaggaaaaagaattgc | |
| | actttaacattaatattgacaaggaggagggcaccacaca | |
| | aaaagttaggtgtaacagaaaatcatgaaactatgattcc | |
| | taatttatatattggaggattttctctaaaaaaaaaaaaa | |
| | tacaacaaataaaaaacactcaatgacctgaccatttgat | |
| | ggagtttaagtcaataccttccctgcaggtgtcctcacag | |
| | gaacgaagtccctaaagaaacagtggcagccaggtttagc | |
| | cccggaattgactggattccttttttagtcattatcaata | |
| | ctcgccatttcaaagaatacgtaaataattaatagtagtg | |
| | attttcctaactttatttagtcaaaaaattagccttttaa | |
| | ttctgctgtaacccgtacatgcccaaaataggggggggtt | |
| | acacagaatatataacatcgtaggtgtctgggtgaacagt | |
| | ttattcctggcatccactaaatataatggagcccgctttt | |
| | taagctggcatccagaaaaaaaaagaatcccagcaccaaa | |
| | atattgttttcttcaccaaccatcagttcataggtccatt | |
| | ctcttagcgcaactacagagaacaggggcacaaacaggca | |
| | aaaaacgggcacaacctcaatggagtgatgcaacctgcct | |
| | ggagtaaatgatgacacaaggcaattgacccacgcatgta | |
| | tctatctcattttcttacaccttctattaccttctgctct | |
| | ctctgatttggaaaaagctgaaaaaaaaggttgaaaccag | |
| | ttccctgaaattattcccctacttgactaataagtatata | |
| | aagacggtaggtattgattgtaattctgtaaatctatttc | |
| | ttaaacttcttaaattctacttttatagttagtctttttt | |
| | ttagttttaaaacaccagaacttagtttcgacggattcta | |
| | gaactagtggatccgttaaattatctaggcattcgattca | |
| | agagatcgaatgcctagataatttaattttttctcgagtc | |
| | atgtaattagttatgtcacgcttacattcacgccctcccc | |
| | ccacatccgctctaaccgaaaaggaaggagttagacaacc | |
| | tgaagtctaggtccctatttatttttttatagttatgtta | |
| | gtattaagaacgttatttatatttcaaatttttctttttt | |
| | ttctgtacagacgcgtgtacgcatgtaacattatactgaa | |
| | aaccttgcttgagaaggttttgggacgctcgaaggcttta | |
| | atttgcgggtcattatcaatactcgccatttcaaagaata | |
| | cgtaaataattaatagtagtgattttcctaactttattta | |
| | gtcaaaaaattagccttttaattctgctgtaacccgtaca | |
| | tgcccaaaataggggggggttacacagaatatataacatc | |
| | gtaggtgtctgggtgaacagtttattcctggcatccacta | |
| | aatataatggagcccgctttttaagctggcatccagaaaa | |
| | aaaaagaatcccagcaccaaaatattgttttcttcaccaa | |
| | ccatcagttcataggtccattctcttagcgcaactacaga | |
| | gaacaggggcacaaacaggcaaaaaacgggcacaacctca | |
| | atggagtgatgcaacctgcctggagtaaatgatgacacaa | |
| | ggcaattgacccacgcatgtatctatctcattttcttaca | |
| | ccttctattaccttctgctctctctgatttggaaaaagct | |
| | gaaaaaaaaggttgaaaccagttccctgaaattattcccc | |
| | tacttgactaataagtatataaagacggtaggtattgatt | |
| | gtaattctgtaaatctatttcttaaacttcttaaattcta | |
| | cttttatagttagtcttttttttagttttaaaacaccaga | |
| | acttagtttcgacggattctagaactagtggatccgttaa | |
| | attatctaggcattcgattcaagagatcgaatgcctagat | |
| | aatttaattttttctcgagtcatgtaattagttatgtcac | |
| | gcttacattcacgccctccccccacatccgctctaaccga | |
| | aaaggaaggagttagacaacctgaagtctaggtccctatt | |
| | tatttttttatagttatgttagtattaagaacgttattta | |
| | tatttcaaatttttcttttttttctgtacagacgcgtgta | |
| | cgcatgtaacattatactgaaaaccttgcttgagaaggtt | |
| | ttgggacgctcgaaggctttaatttgcgggcccattggta | |
| | tggctttttccccgtatccccccaggtgtctgcaggctca | |
| | aagagcagcgagaagcgttcagaggaaagcgatcccgtgc | |
| | caccttccccgtgcccgggctgtccccgcacgctgccggc | |
| | tcggggatgcggggggagcgccggaccggagcggagcccc | |
| | gggcggctcgctgctgccccctagcggggggggacgtaat | |
| | tacatccctgggggctttgggggggggctgtcccttagcg | |
| | gccgctgatatctataacaagaaaatatatatataataag | |
| | ttatcacgtaagtagaacatgaaataacaatataattatc | |
| | gtatgagttaaatcttaaaagtcacgtaaaagataatcat | |
| | gcgtcattttgactcacgcggtcgttatagttcaaaatca | |
| | gtgacacttaccgcattgacaagcacgcctcacgggagct | |
| | ccaagcggcgactgagatgtcctaaatgcacagcgacgga | |
| | ttcgcgctatttagaaagagagagcaatatttcaagaatg | |
| | catgcgtcaattttacgcagactatctttctagggttaa | |
| 9 | gacgaaagggcctcgtgatacgcctatttttataggttaa | pRS426_463 |
| | tgtcatgataataatggtttcttagtatgatccaatatca | plasmid |
| | aaggaaatgatagcattgaaggatgagactaatccaattg | |
| | aggagtggcagcatatagaacagctaaagggtagtgctga | |
| | aggaagcatacgataccccgcatggaatgggataatatca | |
| | caggaggtactagactacctttcatcctacataaatagac | |
| | gcatataagtacgcatttaagcataaacacgcactatgcc | |
| | gttcttctcatgtatatatatatacaggcaacacgcagat | |
| | ataggtgcgacgtgaacagtgagctgtatgtgcgcagctc | |
| | gcgttgcattttcggaagcgctcgttttcggaaacgcttt | |
| | gaagttcctattccgaagttcctattctctagaaagtata | |
| | ggaacttcagagcgcttttgaaaaccaaaagcgctctgaa | |
| | gacgcactttcaaaaaaccaaaaacgcaccggactgtaac | |
| | gagctactaaaatattgcgaataccgcttccacaaacatt | |
| | gctcaaaagtatctctttgctatatatctctgtgctatat | |
| | ccctatataacctacccatccacctttcgctccttgaact | |
| | tgcatctaaactcgacctctacattttttatgtttatctc | |
| | tagtattactctttagacaaaaaaattgtagtaagaacta | |
| | ttcatagagtgaatcgaaaacaatacgaaaatgtaaacat | |
| | ttcctatacgtagtatatagagacaaaatagaagaaaccg | |
| | ttcataattttctgaccaatgaagaatcatcaacgctatc | |
| | actttctgttcacaaagtatgcgcaatccacatcggtata | |
| | gaatataatcggggatgcctttatcttgaaaaaatgcacc | |
| | cgcagcttcgctagtaatcagtaaacgcgggaagtggagt | |
| | caggctttttttatggaagagaaaatagacaccaaagtag | |
| | ccttcttctaaccttaacggacctacagtgcaaaaagtta | |
| | tcaagagactgcattatagagcgcacaaaggagaaaaaaa | |
| | gtaatctaagatgctttgttagaaaaatagcgctctcggg | |
| | atgcatttttgtagaacaaaaaagaagtatagattctttg | |
| | ttggtaaaatagcgctctcgcgttgcatttctgttctgta | |
| | aaaatgcagctcagattctttgtttgaaaaattagcgctc | |
| | tcgcgttgcatttttgttttacaaaaatgaagcacagatt | |
| | cttcgttggtaaaatagcgctttcgcgttgcatttctgtt | |
| | ctgtaaaaatgcagctcagattctttgtttgaaaaattag | |
| | cgctctcgcgttgcatttttgttctacaaaatgaagcaca | |
| | gatgcttcgttcaggtggcacttttcggggaaatgtgcgc | |
| | ggaacccctatttgtttatttttctaaatacattcaaata | |
| | tgtatccgctcatgagacaataaccctgataaatgcttca | |
| | ataatattgaaaaaggaagagtatgagtattcaacatttc | |
| | cgtgtcgcccttattcccttttttgcggcattttgccttc | |
| | ctgtttttgctcacccagaaacgctggtgaaagtaaaaga | |
| | tgctgaagatcagttgggtgcacgagtgggttacatcgaa | |
| | ctggatctcaacagcggtaagatccttgagagttttcgcc | |
| | ccgaagaacgttttccaatgatgagcacttttaaagttct | |
| | gctatgtggcgcggtattatcccgtattgacgccgggcaa | |
| | gagcaactcggtcgccgcatacactattctcagaatgact | |
| | tggttgagtactcaccagtcacagaaaagcatcttacgga | |
| | tggcatgacagtaagagaattatgcagtgctgccataacc | |
| | atgagtgataacactgcggccaacttacttctgacaacga | |
| | tcggaggaccgaaggagctaaccgcttttttgcacaacat | |
| | gggggatcatgtaactcgccttgatcgttgggaaccggag | |
| | ctgaatgaagccataccaaacgacgagcgtgacaccacga | |
| | tgcctgtagcaatggcaacaacgttgcgcaaactattaac | |
| | tggcgaactacttactctagcttcccggcaacaattaata | |
| | gactggatggaggcggataaagttgcaggaccacttctgc | |
| | gctcggcccttccggctggctggtttattgctgataaatc | |
| | tggagccggtgagcgtgggtctcgcggtatcattgcagca | |
| | ctggggccagatggtaagccctcccgtatcgtagttatct | |
| | acacgacggggagtcaggcaactatggatgaacgaaatag | |
| | acagatcgctgagataggtgcctcactgattaagcattgg | |
| | taactgtcagaccaagtttactcatatatactttagattg | |
| | atttaaaacttcatttttaatttaaaaggatctaggtgaa | |
| | gatcctttttgataatctcatgaccaaaatcccttaacgt | |
| | gagttttcgttccactgagcgtcagaccccgtagaaaaga | |
| | tcaaaggatcttcttgagatcctttttttctgcgcgtaat | |
| | ctgctgcttgcaaacaaaaaaaccaccgctaccagcggtg | |
| | gtttgtttgccggatcaagagctaccaactctttttccga | |
| | aggtaactggcttcagcagagcgcagataccaaatactgt | |
| | ccttctagtgtagccgtagttaggccaccacttcaagaac | |
| | tctgtagcaccgcctacatacctcgctctgctaatcctgt | |
| | taccagtggctgctgccagtggcgataagtcgtgtcttac | |
| | cgggttggactcaagacgatagttaccggataaggcgcag | |
| | cggtcgggctgaacggggggttcgtgcacacagcccagct | |
| | tggagcgaacgacctacaccgaactgagatacctacagcg | |
| | tgagctatgagaaagcgccacgcttcccgaagggagaaag | |
| | gcggacaggtatccggtaagcggcagggtcggaacaggag | |
| | agcgcacgagggagcttccagggggaaacgcctggtatct | |
| | ttatagtcctgtcgggtttcgccacctctgacttgagcgt | |
| | cgatttttgtgatgctcgtcaggggggcggagcctatgga | |
| | aaaacgccagcaacgcggcctttttacggttcctggcctt | |
| | ttgctggccttttgctcacatgttctttcctgcgttatcc | |
| | cctgattctgtggataaccgtattaccgcctttgagtgag | |
| | ctgataccgctcgccgcagccgaacgaccgagcgcagcga | |
| | gtcagtgagcgaggaagcggaagagcgcccaatacgcaaa | |
| | ccgcctctccccgcgcgttggccgattcattaatgcagct | |
| | ggcacgacaggtttcccgactggaaagcgggcagtgagcg | |
| | caacgcaattaatgtgagttacctcactcattaggcaccc | |
| | caggctttacactttatgcttccggctcctatgttgtgtg | |
| | gaattgtgagcggataacaatttcacacaggaaacagcta | |
| | tgaccatgattacgccaagcgcgcaattaaccctcactaa | |
| | agggaacaaaagctggagctcagtttatcattatcaatac | |
| | tcgccatttcaaagaatacgtaaataattaatagtagtga | |
| | ttttcctaactttatttagtcaaaaaattagccttttaat | |
| | tctgctgtaacccgtacatgcccaaaataggggggggtta | |
| | cacagaatatataacatcgtaggtgtctgggtgaacagtt | |
| | tattcctggcatccactaaatataatggagcccgcttttt | |
| | aagctggcatccagaaaaaaaaagaatcccagcaccaaaa | |
| | tattgttttcttcaccaaccatcagttcataggtccattc | |
| | tcttagcgcaactacagagaacaggggcacaaacaggcaa | |
| | aaaacgggcacaacctcaatggagtgatgcaacctgcctg | |
| | gagtaaatgatgacacaaggcaattgacccacgcatgtat | |
| | ctatctcattttcttacaccttctattaccttctgctctc | |
| | tctgatttggaaaaagctgaaaaaaaaggttgaaaccagt | |
| | tccctgaaattattcccctacttgactaataagtatataa | |
| | agacggtaggtattgattgtaattctgtaaatctatttct | |
| | taaacttcttaaattctacttttatagttagtcttttttt | |
| | tagttttaaaacaccagaacttagtttcgacggattctag | |
| | aactagtggatccgttaaattatctaggcattcgattcaa | |
| | gagatcgaatgcctagataatttaattttttctcgagtca | |
| | tgtaattagttatgtcacgcttacattcacgccctccccc | |
| | cacatccgctctaaccgaaaaggaaggagttagacaacct | |
| | gaagtctaggtccctatttatttttttatagttatgttag | |
| | tattaagaacgttatttatatttcaaatttttcttttttt | |
| | tctgtacagacgcgtgtacgcatgtaacattatactgaaa | |
| | accttgcttgagaaggttttgggacgctcgaaggctttaa | |
| | tttgcggccggtacccaattcgccctatagtgagtcgtat | |
| | tacgcgcgctcactggccgtcgttttacaacgtcgtgact | |
| | gggaaaaccctggcgttacccaacttaatcgccttgcagc | |
| | acatccccctttcgccagctggcgtaatagcgaagaggcc | |
| | cgcaccgatcgcccttcccaacagttgcgcagcctgaatg | |
| | gcgaatggcgcgacgcgccctgtagcggcgcattaagcgc | |
| | ggcgggtgtggtggttacgcgcagcgtgaccgctacactt | |
| | gccagcgccctagcgcccgctcctttcgctttcttccctt | |
| | cctttctcgccacgttcgccggctttccccgtcaagctct | |
| | aaatcgggggctccctttagggttccgatttagtgcttta | |
| | cggcacctcgaccccaaaaaacttgattagggtgatggtt | |
| | cacgtagtgggccatcgccctgatagacggtttttcgccc | |
| | tttgacgttggagtccacgttctttaatagtggactcttg | |
| | ttccaaactggaacaacactcaaccctatctcggtctatt | |
| | cttttgatttataagggattttgccgatttcggcctattg | |
| | gttaaaaaatgagctgatttaacaaaaatttaacgcgaat | |
| | tttaacaaaatattaacgtttacaatttcctgatgcggta | |
| | ttttctccttacgcatctgtgcggtatttcacaccgcata | |
| | gggtaataactgatataattaaattgaagctctaatttgt | |
| | gagtttagtatacatgcatttacttataatacagtttttt | |
| | agttttgctggccgcatcttctcaaatatgcttcccagcc | |
| | tgcttttctgtaacgttcaccctctaccttagcatccctt | |
| | ccctttgcaaatagtcctcttccaacaataataatgtcag | |
| | atcctgtagagaccacatcatccacggttctatactgttg | |
| | acccaatgcgtctcccttgtcatctaaacccacaccgggt | |
| | gtcataatcaaccaatcgtaaccttcatctcttccaccca | |
| | tgtctctttgagcaataaagccgataacaaaatctttgtc | |
| | gctcttcgcaatgtcaacagtacccttagtatattctcca | |
| | gtagatagggagcccttgcatgacaattctgctaacatca | |
| | aaaggcctctaggttcctttgttacttcttctgccgcctg | |
| | cttcaaaccgctaacaatacctgggcccaccacaccgtgt | |
| | gcattcgtaatgtctgcccattctgctattctgtatacac | |
| | ccgcagagtactgcaatttgactgtattaccaatgtcagc | |
| | aaattttctgtcttcgaagagtaaaaaattgtacttggcg | |
| | gataatgcctttagcggcttaactgtgccctccatggaaa | |
| | aatcagtcaagatatccacatgtgtttttagtaaacaaat | |
| | tttgggacctaatgcttcaactaactccagtaattccttg | |
| | gtggtacgaacatccaatgaagcacacaagtttgtttgct | |
| | tttcgtgcatgatattaaatagcttggcagcaacaggact | |
| | aggatgagtagcagcacgttccttatatgtagctttcgac | |
| | atgatttatcttcgtttcctgcaggtttttgttctgtgca | |
| | gttgggttaagaatactgggcaatttcatgtttcttcaac | |
| | actacatatgcgtatatataccaatctaagtctgtgctcc | |
| | ttccttcgttcttccttctgttcggagattaccgaatcaa | |
| | aaaaatttcaaagaaaccgaaatcaaaaaaaagaataaaa | |
| | aaaaaatgatgaattgaattgaaaagctgtggtatggtgc | |
| | actctcagtacaatctgctctgatgccgcatagttaagcc | |
| | agccccgacacccgccaacacccgctgacgcgccctgacg | |
| | ggcttgtctgctcccggcatccgcttacagacaagctgtg | |
| | accgtctccgggagctgcatgtgtcagaggttttcaccgt | |
| | catcaccgaaacgcgcga | |
| 10 | atgggttcctccttggatgatgaacatattttgtctgcct | Super |
| | tgttgcaatccgatgatgaattggttggtgaagattccga | piggyBac |
| | ttccgaagtttctgatcatgtttccgaagatgatgttcaa | transposase |
| | tctgatactgaagaagccttcatcgacgaagttcatgaag | |
| | ttcaacctacatcttccggttccgaaattttggatgaaca | |
| | aaacgttatcgaacagccaggttcttcattggcttctaac | |
| | agaattttgaccttgccacaaagaaccatcaggggtaaaa | |
| | acaaacattgctggtctacttctaagtccaccagaagatc | |
| | aagagtttctgctttgaacatcgtcagatctcaaaggggt | |
| | ccaactagaatgtgtagaaatatctacgatcccctgttgt | |
| | gcttcaagttgttttttaccgacgaaatcatctccgaaat | |
| | cgttaagtggactaacgccgaaatttccttgaagagaaga | |
| | gaatctatgacctctgctactttcagagatacaaacgaag | |
| | atgagatctacgccttcttcggtattttggttatgactgc | |
| | tgttagaaaggacaaccatatgtctaccgatgatttgttc | |
| | gacaggtctttgtctatggtttacgtgtctgttatgtcca | |
| | gagatagattcgacttcttgatcagatgcttgagaatgga | |
| | tgacaagtctattagaccaaccttgagagaaaacgatgtt | |
| | ttcactccagttagaaagatctgggacttgttcattcatc | |
| | aatgcatccaaaactataccccaggtgctcatttgactat | |
| | tgatgaacaactgttgggtttcagaggtagatgtcctttc | |
| | agagtttacattccaaacaagccatctaagtacggcatca | |
| | agattttgatgatgtgtgactctggtacgaagtacatgat | |
| | taacggtatgccatacttaggtagaggtactcaaactaat | |
| | ggtgttccattgggtgagtactacgtcaaagaattgtcta | |
| | aaccagttcacggttcctgcagaaacattacttgtgataa | |
| | ttggttcacctccattccattggctaagaatttgttgcaa | |
| | gagccatacaagttgaccatcgttggtactgttagatcca | |
| | acaagagagaaatcccagaagtcttgaagaacagtagatc | |
| | aagaccagttggtacttctatgttctgttttgatggtcca | |
| | ttgaccttggtttcttacaaacctaaaccagccaagatgg | |
| | tgtacttgttgtcatcttgtgatgaagatgcctccattaa | |
| | cgaatctactggtaaaccacaaatggtcatgtactacaat | |
| | cagaccaaaggtggtgttgataccttggatcaaatgtgtt | |
| | ctgttatgacctgttccagaaagactaatagatggccaat | |
| | ggctttgttgtacggcatgatcaatattgcctgtatcaac | |
| | tccttcatcatctactcccataacgtttcttctaagggtg | |
| | aaaaggtccagtccagaaaaaagtttatgaggaacctgta | |
| | tatgtccctgacctcttcattcatgagaaagagattggaa | |
| | gctccaactttgaagagatacttgagagacaacatctcca | |
| | acatcttgcctaaagaagttccaggtacatctgatgattc | |
| | aaccgaagaaccagttatgaagaagagaacttactgtacc | |
| | tactgtccctccaaaattagacgtaaagctaacgcttctt | |
| | gtaagaagtgcaagaaggttatctgcagagaacacaacat | |
| | tgacatgtgtcaatcctgcttctag | |
| 11 | tcattatcaatactcgccatttcaaagaatacgtaaataa | glyceraldehyde- |
| | ttaatagtagtgattttcctaactttatttagtcaaaaaa | 3- |
| | ttagccttttaattctgctgtaacccgtacatgcccaaaa | phosphate |
| | taggggggggttacacagaatatataacatcgtaggtgtc | dehydrogenase |
| | tgggtgaacagtttattcctggcatccactaaatataatg | (GAP) |
| | gagcccgctttttaagctggcatccagaaaaaaaaagaat | promoter |
| | cccagcaccaaaatattgttttcttcaccaaccatcagtt | |
| | cataggtccattctcttagcgcaactacagagaacagggg | |
| | cacaaacaggcaaaaaacgggcacaacctcaatggagtga | |
| | tgcaacctgcctggagtaaatgatgacacaaggcaattga | |
| | cccacgcatgtatctatctcattttcttacaccttctatt | |
| | accttctgctctctctgatttggaaaaagctgaaaaaaaa | |
| | ggttgaaaccagttccctgaaattattcccctacttgact | |
| | aataagtatataaagacggtaggtattgattgtaattctg | |
| | taaatctatttcttaaacttcttaaattctacttttatag | |
| | ttagtcttttttttagttttaaaacaccagaacttagttt | |
| | cga | |
| 12 | tcatgtaattagttatgtcacgcttacattcacgccctcc | CYC1 |
| | ccccacatccgctctaaccgaaaaggaaggagttagacaa | terminator |
| | cctgaagtctaggtccctatttatttttttatagttatgt | sequence |
| | tagtattaagaacgttatttatatttcaaatttttctttt | |
| | ttttctgtacagacgcgtgtacgcatgtaacattatactg | |
| | aaaaccttgcttgagaaggttttgggacgctcgaaggctt | |
| | taatttgc | |
| 13 | tctttgaaaagataatgtatgattatgctttcactcatat | SNR52 |
| | ttatacagaaacttgatgttttctttcgagtatatacaag | promoter |
| | gtgattacatgtacgtttgaagtacaactctagattttgt | |
| | agtgccctcttgggctagcggtaaaggtgcgcattttttc | |
| | acaccctacaatgttctgttcaaaagattttggtcaaacg | |
| | ctgtagaagtgaaagttggtgcgcatgtttcggcgttcga | |
| | aacttctccgcagtgaaagataaatgatc | |
| 14 | tttattttttgtcactattgttatgtaaaatgccacctct | SNR52 |
| | gacagtatggaacgcaaacttctgtctagtggataacaga | terminator |
| | atttttctatggccaattta | |
| 15 | aactgtgggaatactcaggtatcgtaagatgcaagagttc | Native |
| | gaatctcttagcaaccattatttttttcctcaacataacg | leucine |
| | agaacacacaggggcgctatcgcacagaatcaaattcgat | promoter |
| | gactggaaattttttgttaatttcagaggtcgcctgacgc | |
| | atatacctttttcaactgaaaaattgggagaaaaaggaaa | |
| | ggtgagaggccggaaccggcttttcatatagaatagagaa | |
| | gcgttcatgactaaatgcttgcatcacaatacttgaagtt | |
| | gacaatattatttaaggacctattgttttttccaataggt | |
| | ggttagcaatcgtcttactttctaacttttcttacctttt | |
| | acatttcagcaatatatatatatatttcaaggatatacca | |
| | ttcta | |
| 16 | EGIKSNISLLKDELRGQISHISHEYLSLIDLAFDSKQNRL | Clo051 |
| | FEMKVLELLVNEYGFKGRHLGGSRKPDGIVYSTTLEDNFG | |
| | IIVDTKAYSEGYSLPISQADEMERYVRENSNRDEEVNPNK | |
| | WWENFSEEVKKYYFVFISGSFKGKFEEQLRRLSMTTGVNG | |
| | SAVNVVNLLLGAEKIRSGEMTIEELERAMENNSEFILKY | |
| 17 | cttgtcatctaaacccacac | URA3 left |
| | | gRNA |
| 18 | gtatagaaccgtggatgatg | URA3 right |
| | | gRNA |
| 19 | gatatcaccaaacatgttgc | LEU2 left |
| | | gRNA |
| 20 | tccgttatcccaggttcctt | LEU2 right |
| | | gRNA |
| 21 | acatgtaggtggcggaggggagatatacaatagaacagat | ADH1 |
| | accagacaagacataatgggctaaacaagactacaccaat | promoter |
| | tacactgcctcattgatggtggtacataacgaactaatac | |
| | tgtagccctagacttgatagccatcatcatatcgaagttt | |
| | cactaccctttttccatttgccatctattgaagtaataat | |
| | aggcgcatgcaacttcttttctttttttttcttttctctc | |
| | tcccccgttgttgtctcaccatatccgcaatgacaaaaaa | |
| | atgatggaagacactaaaggaaaaaattaacgacaaagac | |
| | agcaccaacagatgtcgttgttccagagctgatgaggggt | |
| | atctcgaagcacacgaaactttttccttccttcattcacg | |
| | cacactactctctaatgagcaacggtatacggccttcctt | |
| | ccagttacttgaatttgaaataaaaaaaagtttgctgtct | |
| | tgctatcaagtataaatagacctgcaattattaatctttt | |
| | gtttcctcgtcattgttctcgttccctttcttccttgttt | |
| | ctttttctgcacaatatttcaagctataccaagcatacaa | |
| | tcaactatctcatataca | |
| 22 | atcggttctttccaatttttttttttttttt tttttttt | DMT9- |
| | ttttttttttggttgtaata | 52.2R #3 5′ |
| | | 60 bp |
| 23 | aagaataaacatttatctgatatgtaattgcatttataaa | DMT9- |
| | atgtacagtaccgcatttaa | 52.2R #3 IV |
| | | 3′ 60 bp |
| 24 | aagaaatatatagatttaggtattcgttaaatatatacac | DMT9- |
| | attaaatggcctcagaaatt | 52.2R #3 |
| | | VIII 5′ 60 bp |
| 25 | atatataaataaataagctcttatatgtacaaatttgtgc | DMT9- |
| | atatacttttcttgaccttt | 52.2R #3 |
| | | VIII 3′ 60 bp |
| 26 | agtctaagctgaaagattattactttcatttgattttttt | DMT9- |
| | atttttgaagccccatttcc | 52.2R #3 X |
| | | 5′ 60 bp |
| 27 | atcgttctcgtggacgagattaaaaatagaaatgatgtag | DMT9- |
| | aggagatgcactaaacattg | 52.2R #3 X |
| | | 3′ 60 bp |
| 28 | taacaattgataaattatttgaagtatcttccaagacttc | DMT9- |
| | aaacaaagatattttcaagt | 52.2R #3 XI |
| | | 5′ 60 bp |
| 29 | aaaggttgtgaagtcaactgttcaagacatgactggcaaa | DMT9- |
| | ggaaactttatgcatctatc | 52.2R #3 XI |
| | | 3′ 60 bp |
| 30 | tttttcttggcctcgaagaaatttcgagataccttgctcg | DMT9- |
| | taaccctcccagaagtttcc | 52.2R #3 XII |
| | | 5′ 60 bp |
| 31 | tattactgtagtccccacgggacaagataccttgtacctt | DMT9- |
| | tttccattggtaataccacc | 52.2R #3 XII |
| | | 3′ 60 bp |
| 32 | gaataacggaaaaggagcctgcagccagactgtagaaaga | DMT9- |
| | tgacactgccaagagaataa | 56.10R #3 |
| | | IV 5′ 60 bp |
| 33 | aagaaaaaacaccccaaacaccctgaccggcggcgaagcc | DMT9- |
| | cctctgcgcgctcaacgcgt | 56.10R #3 |
| | | IV 3′ 60 bp |

DOCID: DOCPROPERTY DOCXDOCID
DMS = IManage
Format = <<LIB>>.<<NUM>>.<<VER>>

I claim:

1. A prototrophic mutant yeast cell comprising an expression cassette stably integrated into at least one location within the genome of the yeast cell,
wherein the expression cassette comprises a nucleotide sequence encoding an interfering RNA molecule; and wherein the interfering RNA molecule is capable of inhibiting gene expression of Shaker in a mosquito; and
wherein the nucleotide sequence encoding the interfering RNA molecule is flanked by an inverted terminal repeat sequence.

2. The yeast cell of claim 1, wherein the nucleotide sequence encoding the interfering RNA molecule is flanked by a 5′ inverted terminal repeat sequence and a 3′ inverted terminal repeat sequence,
wherein the 5′ inverted terminal repeat sequence has at least about 70% identity to the entire length of SEQ ID NO:5; and
wherein the 3′ inverted terminal repeat sequence has at least about 70% identity to the entire length of SEQ ID NO:4.

3. The yeast cell of claim 1, wherein the nucleotide sequence encoding the interfering RNA molecule is operably linked to a glyceraldehyde-3-phosphate dehydrogenase (GAP) promoter, wherein the GAP promoter separates the nucleotide sequence encoding the interfering RNA molecule from the 5′ inverted terminal repeat sequence.

4. The yeast cell of claim 3, wherein the nucleotide sequence encoding the interfering RNA molecule is flanked on the 3′ end by a CYC1 terminator, wherein the CYC1 terminator separates the nucleotide sequence encoding the interfering RNA molecule from the 3′ inverted terminal repeat sequence.

5. The yeast cell of claim 4, wherein the GAP promoter comprises a nucleotide sequence with at least 85% sequence identity to SEQ ID NO:11 and the CYC1 terminator comprises a nucleotide sequence with at least 85% sequence identity to SEQ ID NO:12.

6. The yeast cell of claim 1, wherein the expression cassette further comprises an auxotrophic rescue promoter operably linked to an auxotrophic rescue gene, and wherein the auxotrophic rescue promoter is flanked by the 5′ inverted terminal repeat sequence.

7. The yeast cell of claim 6, wherein the auxotrophic rescue promoter comprises a minimal auxotrophic rescue promoter, wherein the minimal auxotrophic rescue promoter comprises a nucleotide sequence with at least 70% identity to the entire length of SEQ ID NO:3.

8. The yeast cell of claim 1, wherein the auxotrophic rescue gene encodes leucine.

9. The yeast cell of claim 1, wherein the expression cassette further comprises an insulator sequence, the insulator sequence separating the 5′ end of the nucleotide sequence encoding the interfering RNA molecule from the 5′ inverted terminal repeat sequence; and the insulator sequence separates the 3′ end of the nucleotide sequence encoding the interfering RNA molecule from the 3′ inverted terminal repeat sequence, the insulator sequence has 70% identity to the entire length of SEQ ID NO:6.

10. The yeast cell of claim 1, wherein the yeast cell has a complemented auxotrophic mutation, the complemented auxotrophic mutation comprising his3Δ0, leu2Δ0, trp1Δ0, ura3Δ0, or a combination thereof.

11. The yeast cell of claim 1, wherein the expression cassette comprises at least one, at least three, or at least five copies of the nucleotide sequence encoding the interfering RNA molecule, the expression cassette comprising one, two, three, four, five, six, seven, eight, nine, or ten locations of integration into the yeast cell genome.

12. The yeast cell of claim 1, wherein a nucleotide sequence transcribed from the nucleotide sequence encoding the interfering RNA molecule comprises at least 25 contiguous nucleotides which are partially or perfectly complementary to a sequence comprising at least 80%, 84%, 88%, 92%, 96%, or 100% identity to the entire length of SEQ ID NO:2.

13. The yeast cell of claim 1, wherein the nucleotide sequence encoding the interfering RNA molecule has at least 80% or at least 90% sequence similarity to the entire length of SEQ ID NO. 1.

14. The yeast cell of claim 1, wherein the expression cassette comprises a nucleotide sequence with at least 70%, 80%, or 90% identity to the entire length of:
a) SEQ ID NO:7 or the complement thereof, or
b) SEQ ID NO:8 or the complement thereof.

15. The yeast cell of claim 1, wherein the yeast cell is Pichia pastoris, Saccharomyces cerevisiae, or Yarrowia lipolytica.

16. The yeast cell of claim 1, wherein the mosquito is a species of Aedes, Anopheles, or Culex.

17. The yeast cell of claim 1, wherein 72 hours of growth in a high-cell density fermentation media yields at least 15 grams per liter of dry cell weight.

18. A composition comprising the yeast cell of claim 1, wherein the yeast cell is heat-killed and/or lyophilized, the composition further comprising a sugar bait or an insecticide.

19. The composition of claim 18, wherein the composition is selectively insecticidal to a mosquito,
wherein the mosquito is a larval mosquito or an adult mosquito; and
wherein the mosquito is a species of Aedes, Anopheles, or Culex.

20. A method for controlling a mosquito population, comprising feeding a mosquito with the yeast cell of claim 1,
wherein the mosquito is a larval mosquito or an adult mosquito; and
wherein the mosquito is a species of Aedes, Anopheles, or Culex, thereby controlling the mosquito population.
